(19) United States
(12) Patent Application Publication
(10) Pub. No.: US 2025/0163064 A1
(43) Pub. Date: May 22, 2025

(54) QUINAZOLINE COMPOUNDS AND USES THEREOF AS INHIBITORS OF MUTANT KRAS PROTEINS

(71) Applicant: AMGEN INC., Thousand Oaks, CA (US)

(72) Inventors: Brian Alan Lanman, Woodland Hills, CA (US); Ryan Paul Wurz, Newbury Park, CA (US); Wei Zhao, Westford, MA (US); Xiaofen LI, Thousand Oaks, CA (US); Michael YAMANO, Thousand Oaks, CA (US); Imelda HOT, Thousand Oaks, CA (US); Rene RAHIMOFF, Santa Monica, CA (US); Liping Pettus, Thousand Oaks, CA (US); Jose Medina, Thousand Oaks, CA (US)

(73) Assignee: AMGEN INC., Thousand Oaks, CA (US)

(21) Appl. No.: 18/838,954

(22) PCT Filed: Feb. 15, 2023
(86) PCT No.: PCT/US2023/062687

§ 371 Date: Aug. 15, 2024

Related U.S. Application Data
(60) Provisional application No. 63/310,915, filed on Feb. 16, 2022.

(51) Int. Cl. C07D 487/04; A61K 31/517; A61K 31/519; A61K 31/55; A61K 31/553; A61K 31/554; C07D 519/00

(52) U.S. Cl. CPC C07D 487/04; A61K 31/517; A61K 31/519; A61K 31/55; A61K 31/553; A61K 31/554; C07D 519/00

(57) ABSTRACT

The present disclosure provides compounds useful for the inhibition of KRAS G12D, G12V, G12A, G12S or G12C. The compounds have a general Formula I: (I) wherein the variables of Formula I are defined herein. This disclosure also provides pharmaceutical compositions comprising the compounds, uses of the compounds, and compositions for treatment of, for example, cancer.

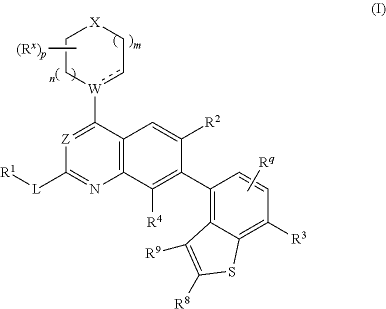

### FIELD

The present disclosure provides compounds having activity as inhibitors of mutant KRAS proteins. This disclosure also provides pharmaceutical compositions comprising the compounds, uses and methods of treating certain disorders, such as cancer, including but not limited to non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

### BACKGROUND

From its identification as one of the first human oncogenes in 1982 (Der et al., 1982), KRAS (the Kirsten rat sarcoma viral oncogene homologue) has been the focus of extensive academic and industrial research, as a key node in the MAPK signal transduction pathway, as a transforming factor in a network of parallel effector pathways (e.g., PI3K/AKT) (Vojtek et al., 1998) and as a potential target for anti-cancer agents (Malumbres et al., 2003). Despite progress in the development of inhibitors of upstream and downstream nodes in the MAPK pathway (e.g., EGFR (Sridhar et al., 2003), BRAF (Holderfield et al., 2014) and MOK (Caunt et al., 2015), the KRAS protein has historically proven resistant to direct inhibition.

KRAS is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins have historically been thwarted by the absence of druggable pockets on the surface of the protein (Cox et al., 2014). In 2013, Shokat and colleagues identified covalent inhibitors of a common (O'Bryan, 2019) oncogenic mutant of KRAS, KRAS G12C, which bound to a previously unrecognized allosteric pocket on GDP-KRAS G12C and prevented its subsequent activation (Ostream et al., 2013). This discovery brought about significant new efforts in the KRAS inhibitor research, which have recently culminated in the entry of KRAS inhibitors in human clinical trials.

While some progress has been made on KRAS G12C inhibitors, there is a continued interest and effort to develop inhibitors of KRAS, particularly inhibitors of other KRAS such as KRAS G12D, G12V, G12A or G12S. Thus, there is a need to develop new inhibitors for KRAS G12D, G12V, G12A, G12S or G12C for the treatment of disorders, such as cancer.

### SUMMARy

In one aspect, the present application is directed to a compound of formula (I);

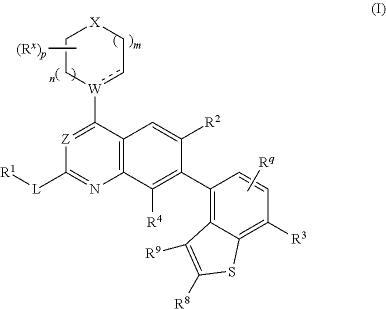

or tautomer thereof, or a pharmaceutically acceptable salt of said compound or said tautomer, wherein;
[symbol] is a single bond or a double bond;
W is C, CH or N, wherein when W is N, [symbol] is a single bond;
X is a bond, CH₂, O, S, S(O), S(O)(NR²) or S(O)₂;
n is 0, 1, 2, or 3;
m is 0, 1, 2, 3 or 4;
each R^{x} is hydroxyl, halogen, oxo, cyano, C₁₋₄ alkyl, C₁₋₄ haloalkyl, C₁₋₄ alkoxy, —T—R^{y} or two R^{x} taken together with the same carbon or adjacent carbon atoms can form C₃₋₇ cycloalkyl or a 5-7 membered heterocycloalkyl, wherein each C₃₋₇ cycloalkyl or 5-7 membered heterocycloalkyl is further substituted with 0-3 occurrences of R^{y} or two R^{x} taken together with the same carbon atom can form a C₃₋₈ cycloalkyl or 4-7 membered heterocycloalkyl substituted with 0-3 occurrences of R^{y} or two R^{x} taken together with the carbon atoms to which they are attached can form a bridged ring, wherein the bridge atoms are selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—;
Z is CH, CR′ or N;
R′ is halogen, cyano or C₁₋₄ alkyl;
L is a bond, —C₁₋₄ alkylene, —O—C₁₋₄ alkylene, —S—C₁₋₄ alkylene, —NR^{z}—, —O— or —S—;
R¹ is hydroxyl, aryl, heteroaryl, C₃₋₈ cycloalkyl or heterocycloalkyl optionally substituted with 0-3 occurrences of R⁵;
R² is hydrogen, halogen, C₁₋₄ alkyl, C₂₋₄ alkenyl or cyano;
R³ hydrogen, halogen, cyano, C₁₋₄ alkyl, C₁₋₄ haloalkyl or C₂₋₄ alkynyl;
R⁴ is hydrogen or halogen;
each R⁵ is halogen, oxo, hydroxyl, amino or C₁₋₄ alkyl;
p is 0, 1, 2 or 3;
R⁸ is amino;
R⁹ is cyano;
T is C₁₋₄ alkylene, —C(O)—, —O—, —S(O)₂— or —S—;
R^{q} is hydrogen, halogen or C₁₋₄ alkyl;
R^{y} is halogen, hydroxyl, oxo, cyano or amino; and
R^{z} is hydrogen or C₁₋₄ alkyl.

In a second aspect, provided herein is a pharmaceutical composition comprising a compound of Formula I or a pharmaceutically acceptable salt of said compound and a pharmaceutically acceptable excipient.

In a third aspect, provided herein is a compound of Formula I, or a pharmaceutically acceptable salt of said compound, or the pharmaceutical composition as described herein for use in treating cancer.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims,

### DETAILED DESCRIPTION

Provided herein as embodiment 1 is a compound of formula (I):

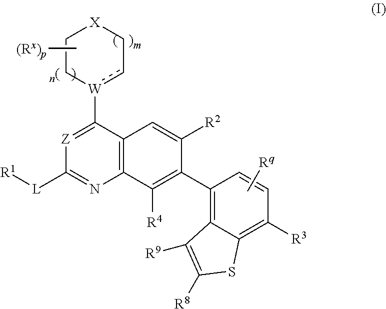

or tautomer thereof, or a pharmaceutically acceptable salt of said compound or said tautomer, wherein;
[symbol] is a single bond or a double bond;
W is C, CH or N, wherein when W is N, [symbol] is a single bond;
X is a bond, CH₂, O, S, S(O), S(O)(NR⁷) or S(O)₂;
n is 0, 1, 2, or 3;
m is 0, 1, 2, 3 or 4;
each R is hydroxyl, halogen, oxo, cyano, C₁₋₄ alkyl, C₁₋₄ haloalkyl, C₁₋₄ alkoxy, —T—R^{y} or two R^{x} taken together with the same carbon or adjacent carbon atoms can form C₃₋₇ cycloalkyl or a 5-7 membered heterocycloalkyl, wherein each C₃₋₇ cycloalkyl or 5-7 membered heterocycloalkyl is further substituted with 0-3 occurrences of R^{y} or two R^{x} taken together with the same carbon atom can form a C₃₋₈ cycloalkyl or 4-7 membered heterocycloalkyl substituted with 0-3 occurrences of R^{y} or two R^{x} taken together with the carbon atoms to which they are attached can form a bridged ring, wherein the bridge atoms are selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—;
Z is CH, CR′ or N;
R′ is halogen, cyano or C₁₋₄ alkyl;
L is a bond, —C₁₋₄ alkylene, —O—C₁₋₄ alkylene, —S—C₁₋₄ alkylene, —NR²—, —O— or —S—;
R¹ is hydroxyl, aryl, heteroaryl, C₃₋₈ cycloalkyl or heterocycloalkyl optionally substituted with 0-3 occurrences of R⁵;
R² is hydrogen, halogen, C₁₋₄ alkyl, C₂₋₄ alkenyl or cyano;
R³ hydrogen, halogen, cyano, C₁₋₄ alkyl, C₁₋₄ haloalkyl or C₂₋₄ alkynyl;
R⁴ is hydrogen or halogen;
each R⁵ is halogen, oxo, hydroxyl, amino or C₁₋₄ alkyl;
p is 0, 1, 2 or 3;
R⁸ is amino;
R⁹ is cyano;
T is C₁₋₄ alkylene, —C(O)—, —O—, —S(O)₂— or —S—;
R^{q} is hydrogen, halogen or C₁₋₄ alkyl;
R^{y} is halogen, hydroxyl, oxo, cyano or amino; and
R^{z} is hydrogen or C₁₋₄ alkyl.

Provided herein as embodiment 2 is the compound according to embodiment 1, wherein Z is N.

Provided herein as embodiment 3 is the compound according to any one of embodiments 1-2, wherein L is —C₁₋₄ alkylene or —O—C₁₋₄ alkylene. Provided herein as embodiment 4 is the compound according to embodiment 3, wherein L is —C₁₋₄ alkylene (e.g., -methylene). Provided herein as embodiment 5 is the compound according to embodiment 3, wherein L is —O—C₁₋₄ alkylene (e.g., —O-methylene or —O-2,2-dimethylethylene). Provided herein as embodiment 6 is the compound according to embodiment 5, wherein L is-O-methylene.

Provided herein as embodiment 7 is the compound according to embodiment 6, wherein R¹ is 7-(hexahydro-1H-pyrrolizine) substituted with 0-3 occurrences of R⁵. Provided herein as embodiment 8 is the compound according to embodiment 7, wherein R¹ is 7-(hexahydro-1H-pyrrolizine) substituted with one occurrence of R³. Provided herein as embodiment 9 is the compound according to embodiment 8, wherein R⁵ is halogen (e.g., fluorine). Provided herein as embodiment 10 is the compound according to embodiment 7, wherein R¹ is 7-(hexahydro-1H-pyrrolizine) substituted with 2 occurrences of R⁵. Provided herein as embodiment 11 is the compound according to embodiment 10, wherein both R⁵ are halogen (e.g., fluorine).

Provided herein as embodiment 12 is the compound according to embodiment 6, wherein R¹ is 2-pyrrolidine substituted with 0-3 occurrences of R⁵. Provided herein as embodiment 13 is the compound according to embodiment 12, wherein R¹ is 2-pyrrolidine substituted with 2 occurrences of R⁵. Provided herein as embodiment 14 is the compound according to embodiment 13, wherein one R⁵ is C₁₋₄ alkyl (e.g., methyl) and the other R⁵ is halogen (e.g., fluorine).

Provided herein as embodiment 15 is the compound according to embodiment 5, wherein L is —O-isopentanylene (i.e., —O-2,2-dimethylethylene). Provided herein as embodiment 16 is the compound according to embodiment 15, wherein R¹ is hydroxyl.

Provided herein as embodiment 17 is the compound according to embodiment 4, wherein L is-methylene. Provided herein as embodiment 18 is the compound according to embodiment 17, wherein R¹ is N-morpholinyl substituted with 0-3 occurrences of R⁵. Provided herein as embodiment 19 is the compound according to embodiment 18, wherein R is N-morpholinyl substituted with 0 occurrences of R⁵.

Provided herein as embodiment 20 is the compound according to embodiments 1-19, wherein L-R¹ is

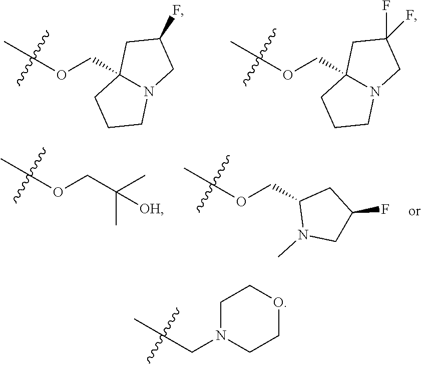

Provided herein as embodiment 21 is the compound according to embodiment 20, wherein L—R¹ is

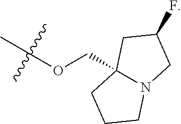 [SMILES: *OC[C@@]12CCCN1C[C@H](F)C2]

Provided herein as embodiment 22 is the compound according to embodiment 20, wherein L—R¹ is

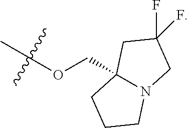 [SMILES: *OC[C@@]12CCCN1CC(F)(F)C2]

Provided herein as embodiment 23 is the compound according to embodiment 20, wherein L—R¹ is

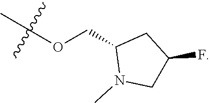 [SMILES: *OC[C@@H]1C[C@@H](F)CN1C]

Provided herein as embodiment 24 is the compound according to embodiment 20, wherein L-R¹ is

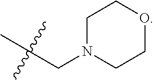 [SMILES: *CN1CCOCC1]

Provided herein as embodiment 25 is the compound according to embodiment 20, wherein L—R¹ is

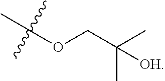 [SMILES: *OCC(C)(C)O]

Provided herein as embodiment 26 is the compound according to any one of embodiments 1-25, wherein W is N.

Provided herein as embodiment 27 is the compound according to embodiment 26, wherein X is O. Provided herein as embodiment 28 is the compound according to embodiment 27, wherein n is 1 and m is 2 or m is 1 and n is 2. Provided herein as embodiment 29 is the compound according to embodiment 28, wherein p is 0.

Provided herein as embodiment 30 is the compound according to embodiment 28, wherein p is 1. Provided herein as embodiment 31 is the compound according to embodiment 30, wherein R^{x} is hydroxyl, oxo, halogen or —T—R^{y}. Provided herein as embodiment 32 is the compound according to embodiment 31, wherein R^{x} is hydroxyl, oxo, fluorine or —SO₂NH₂. Provided herein as embodiment 33 is the compound according to embodiment 32, wherein R^{x} is hydroxyl, oxo or fluorine.

Provided herein as embodiment 34 is the compound according to embodiment 28, wherein p is 2. Provided herein as embodiment 35 is the compound according to embodiment 34, wherein each R¹ is hydroxyl, halogen, C₁₋₄ alkyl, C₁₄ haloalkyl or two R^{x} are taken together with the carbon atoms to which they are attached to form a bridged ring with the bridge atoms are-C₁₋₄ alkylene. Provided herein as embodiment 36 is the compound according to embodiment 35, wherein one R^{x} is hydroxyl and the other R^{x} is C₁₋₄ alkyl (e.g., methyl or ethyl). Provided herein as embodiment 37 is the compound according to embodiment 35, wherein both R^{x} are halogen (e.g., fluorine). Provided herein as embodiment 38 is the compound according to embodiment 35, wherein one R¹ is hydroxyl and the other R^{x} is C₁₋₄ haloalkyl (e.g., difluoromethyl). Provided herein as embodiment 39 is the compound according to embodiment 35, wherein two R^{x} are taken together with the carbon atoms to which they are attached to form a bridged ring wherein the bridge atoms are methylene or ethylene.

Provided herein as embodiment 40 is the compound according to embodiment 27, wherein n is 2 and m is 2. Provided herein as embodiment 41 is the compound according to embodiment 40, wherein p is 0. Provided herein as embodiment 42 is the compound according to embodiment 27, wherein m is 1 and n is 3. Provided herein as embodiment 43 is the compound according to embodiment 42, wherein p is 0.

Provided herein as embodiment 44 is the compound according to embodiment 26, wherein X is S(O)₂. Provided herein as embodiment 45 is the compound according to embodiment 44, wherein n is 1 and m is 2 or m is 1 and n is 2. Provided herein as embodiment 46 is the compound according to embodiment 45, wherein p is 0.

Provided herein as embodiment 47 is the compound according to any one of embodiments 26-46, wherein

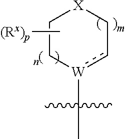

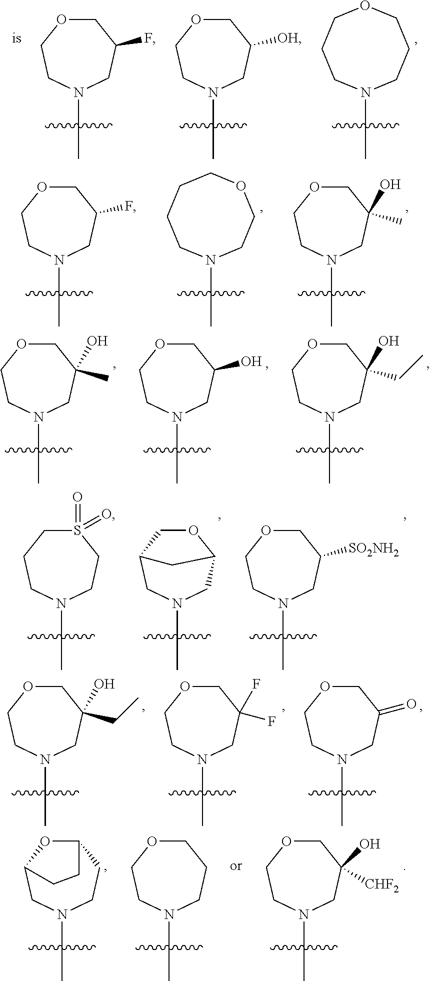

Provided herein as embodiment 48 is the compound according to embodiment 47, wherein

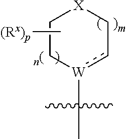

is

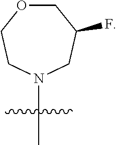 [SMILES: *N1CCOC[C@@H](F)C1]

Provided herein as embodiment 49 is the compound according to embodiment 47, wherein

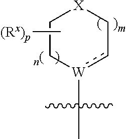

is

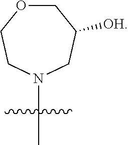 [SMILES: *N1CCOC[C@H](O)C1]

Provided herein as embodiment 50 is the compound according to embodiment 47, wherein

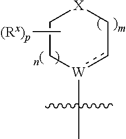

is

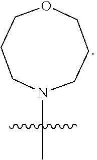 [SMILES: *N1CCCOCCC1]

Provided herein as embodiment 51 is the compound according to embodiment 47, wherein

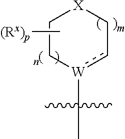

is

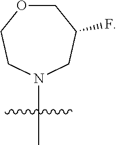 [SMILES: *N1CCOC[C@H](F)C1]

Provided herein as embodiment 52 is the compound according to embodiment 47, wherein

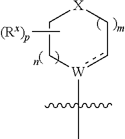

is

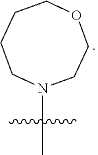 [SMILES: *N1CCCCOCC1]

Provided herein as embodiment 53 is the compound according to embodiment 47, wherein

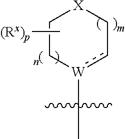

is

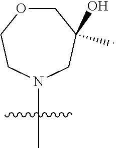 [SMILES: *N1CCOC[C@@](C)(O)C1 ; "."]

Provided herein as embodiment 54 is the compound according to embodiment 47, wherein

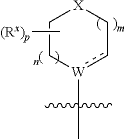

is

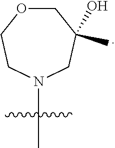 [SMILES: *N1CCOC[C@](C)(O)C1 ; "."]

Provided herein as embodiment 55 is the compound according to embodiment 47, wherein

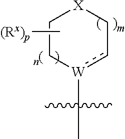

is

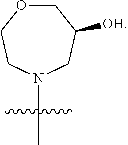 [SMILES: *N1CCOC[C@@H](O)C1]

Provided herein as embodiment 56 is the compound according to embodiment 47, wherein

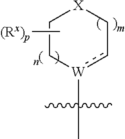

is

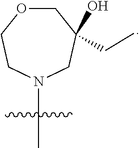 [SMILES: *N1CCOC[C@](O)(CC)C1 ; "."]

Provided herein as embodiment 57 is the compound according to embodiment 47, wherein

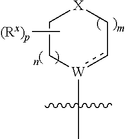

is

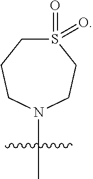 [SMILES: *N1CCCS(=O)(=O)CC1]

Provided herein as embodiment 58 is the compound according to embodiment 47, wherein

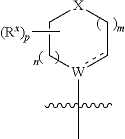

is

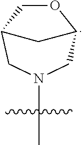 [SMILES: *N1C[C@@H]2CO[C@@H](C2)C1 ; "."]

Provided herein as embodiment 59 is the compound according to embodiment 47, wherein

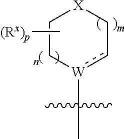

is

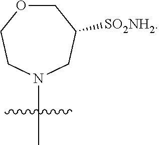 [SMILES: *N1CCOC[C@H](S(N)(=O)=O)C1]

Provided herein as embodiment 60 is the compound according to embodiment 47, wherein

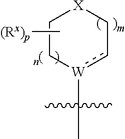

is

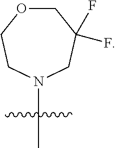 [SMILES: *N1CCOCC(F)(F)C1]

Provided herein as embodiment 61 is the compound according to embodiment 47, wherein

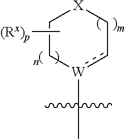

is

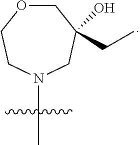 [SMILES: *N1CCOC[C@@](O)(CC)C1 ; "."]

Provided herein as embodiment 62 is the compound according to embodiment 47, wherein

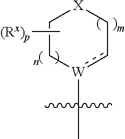

is

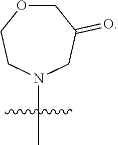 [SMILES: *N1CCOCC(=O)C1]

Provided herein as embodiment 63 is the compound according to embodiment 47, wherein

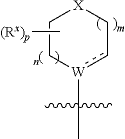

is

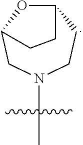 [SMILES: *N1CC[C@@H]2CC[C@H](C1)O2 ; "."]

Provided herein as embodiment 64 is the compound according to embodiment 47, wherein

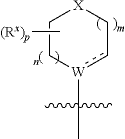

is

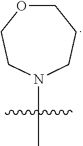 [SMILES: *N1CCCOCC1 ; "."]

Provided herein as embodiment 65 is the compound according to embodiment 47, wherein

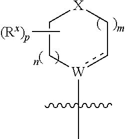

is

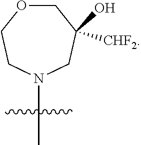 [SMILES: *N1CCOC[C@](O)(C(F)F)C1]

Provided herein as embodiment 66 is the compound according to embodiment 26, wherein X is CH₂. Provided herein as embodiment 67 is the compound according to embodiment 66, wherein n is 0 and m is 1 or m is 0 and n is 1.

Provided herein as embodiment 68 is the compound according to embodiment 67, wherein p is 1. Provided herein as embodiment 69 is the compound according to embodiment 68, wherein R^{x} is C₁₋₄ alkyl, cyano or —T—R^{y}. Provided herein as embodiment 70 is the compound according to embodiment 69, wherein R^{x} is methyl, cyano, —CH₂OH, —CH₂CN or —CH₂OMe.

Provided herein as embodiment 71 is the compound according to embodiment 67, wherein p is 2.

Provided herein as embodiment 72 is the compound according to embodiment 71, wherein each R^{x} is C₁₋₄ alkyl, hydroxyl or —T—R^{Y} or two R^{x} are taken together with the same carbon atom to form a 4-7 membered heterocycloalkyl substituted with 0-3 occurrences of R^{y}. Provided herein as embodiment 73 is the compound according to embodiment 72, wherein one R^{x} is methyl and the other R¹ is —CH₂OH. Provided herein as embodiment 74 is the compound according to embodiment 72, wherein one R^{x} is methyl and the other R^{x} is hydroxyl, Provided herein as embodiment 75 is the compound according to embodiment 72, wherein both R^{x} are taken together with the same carbon atom to form a 2-tetrahydrothiophene substituted with 2 occurrences of R^{y}. Provided herein as embodiment 76 is the compound according to embodiment 75, wherein both R^{y} are oxo,

Provided herein as embodiment 77 is the compound according to any one of embodiments 66-76, wherein

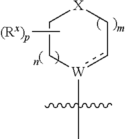

is

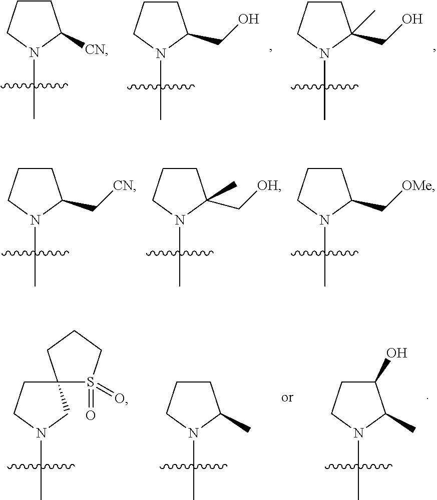

Provided herein as embodiment 78 is the compound according to embodiment 77, wherein

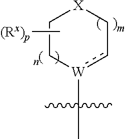

is

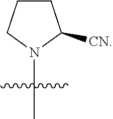 [SMILES: *N1CCC[C@H]1C#N]

Provided herein as embodiment 79 is the compound according to embodiment 77, wherein

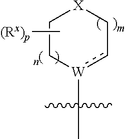

is

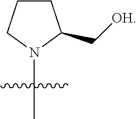 [SMILES: *N1CCC[C@H]1CO]

Provided herein as embodiment 80 is the compound according to embodiment 77, wherein

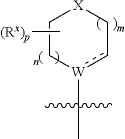

is

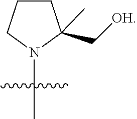 [SMILES: *N1CCC[C@@]1(C)CO]

Provided herein as embodiment 81 is the compound according to embodiment 77, wherein

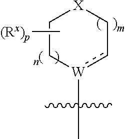

is

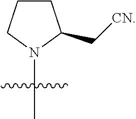 [SMILES: *N1CCC[C@H]1CC#N]

Provided herein as embodiment 82 is the compound according to embodiment 77, wherein

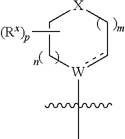

is

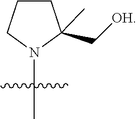 [SMILES: *N1CCC[C@@]1(C)CO]

Provided herein as embodiment 83 is the compound according to embodiment 77, wherein

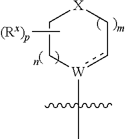

is

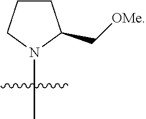 [SMILES: *N1CCC[C@H]1COC]

Provided herein as embodiment 84 is the compound according to embodiment 77, wherein

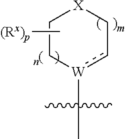

is

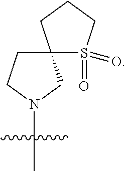 [SMILES: *N1CC[C@]2(CCCS2(=O)=O)C1]

Provided herein as embodiment 85 is the compound according to embodiment 77, wherein

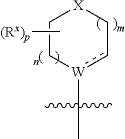

is

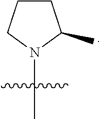 [SMILES: *N1CCC[C@H]1C ; "."]

Provided herein as embodiment 86 is the compound according to embodiment 77, wherein

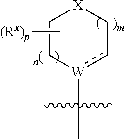

is

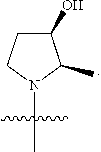 [SMILES: *N1CC[C@@H](O)[C@H]1C ; "."]

Provided herein as embodiment 87 is the compound according to embodiment 66, wherein n is 1 and m is 1. Provided herein as embodiment 88 is the compound according to embodiment 87, wherein p is 0. Provided herein as embodiment 89 is the compound according to embodiment 87, wherein p is 1. Provided herein as embodiment 90 is the compound according to embodiment 89, wherein R^{x} is hydroxyl, cyano or —T—R^{y}. Provided herein as embodiment 91 is the compound according to embodiment 90, wherein R^{x} is hydroxyl, cyano or —S(O)₂—NH₂.

Provided herein as embodiment 92 is the compound according to embodiment 87, wherein p is 2. Provided herein as embodiment 93 is the compound according to embodiment 92, wherein each R^{x} is hydroxyl, C₁₋₄ alkyl, ˜T—R^{y} or two R^{x} are taken together with the same carbon atom to form a C₃₋₇ cycloalkyl or 4-7 membered heterocycloalkyl substituted with 0-3 occurrences of R^{y}. Provided herein as embodiment 94 is the compound according to embodiment 93, wherein one R^{x} is hydroxyl and the other R^{x} is methyl. Provided herein as embodiment 95 is the compound according to embodiment 93, wherein one R^{x} is hydroxyl and the other R^{x} is ethyl. Provided herein as embodiment 96 is the compound according to embodiment 93, wherein one R^{x} is methyl and the other R^{x} is —CH₂OH. Provided herein as embodiment 97 is the compound according to embodiment 93, wherein one R^{x} is methyl and the other R^{x} is —C(O)NH₂.

Provided herein as embodiment 98 is the compound according to embodiment 93, wherein two R^{x} are taken together with the same carbon atom to form a cyclobutyl substituted with one occurrence of R^{y}. Provided herein as embodiment 99 is the compound according to embodiment 98, wherein R^{y} is oxo or hydroxyl. Provided herein as embodiment 100 is the compound according to embodiment 93, wherein two R^{x} are taken together with the same carbon atom to form a 2-azetidinyl substituted with one occurrence of R^{y}. Provided herein as embodiment 101 is the compound according to embodiment 100, wherein R^{y} is oxo.

Provided herein as embodiment 102 is the compound according to embodiment 93, wherein two R^{x} are taken together with the same carbon atom to form a 3-azetidinyl substituted with one occurrence of —T—R^{y}. Provided herein as embodiment 103 is the compound according to embodiment 102, wherein —T—R^{y} is —SO₂-Me.

Provided herein as embodiment 104 is the compound according to embodiment 93, wherein two R^{x} are taken together with the same carbon atom to form a 5-oxazolidinyl substituted with one occurrence of R^{y}. Provided herein as embodiment 105 is the compound according to embodiment 104, wherein R^{y} is oxo.

Provided herein as embodiment 106 is the compound according to embodiment 93, wherein two R^{x} are taken together with the same carbon atom to form a 3-oxetanyl substituted with 0 occurrences of R^{y}. Provided herein as embodiment 107 is the compound according to embodiment 93, wherein two R^{x} are taken together with the same carbon atom to form a 2-oxetanyl substituted with 0 occurrences of R^{y}.

Provided herein as embodiment 108 is the compound according to embodiment 93, wherein two R^{x} are taken together with the same carbon atom to form a 3-tetrahydrothiophenyl substituted with two occurrences of R^{y}. Provided herein as embodiment 109 is the compound according to embodiment 108, wherein both R^{y} are oxo.

Provided herein as embodiment 110 is the compound according to embodiment 93, wherein two R^{x} are taken together with the same carbon atom to form a 2-thietanyl substituted with two occurrences of R^{y}. Provided herein as embodiment 111 is the compound according to embodiment 110, wherein both R^{y} are oxo.

Provided herein as embodiment 112 is the compound according to embodiment 93, wherein two R^{x} are taken together with the same carbon atom to form a 2-tetrahydrofuranyl substituted with 0 occurrences of R^{y}. Provided herein as embodiment 113 is the compound according to embodiment 93, wherein two R^{x} are taken together with the same carbon atom to form a 3-tetrahydrofuranyl substituted with 0 occurrences of R^{y}.

Provided herein as embodiment 114 is the compound according to any one of embodiments 87-113, wherein

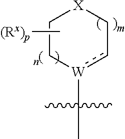

is

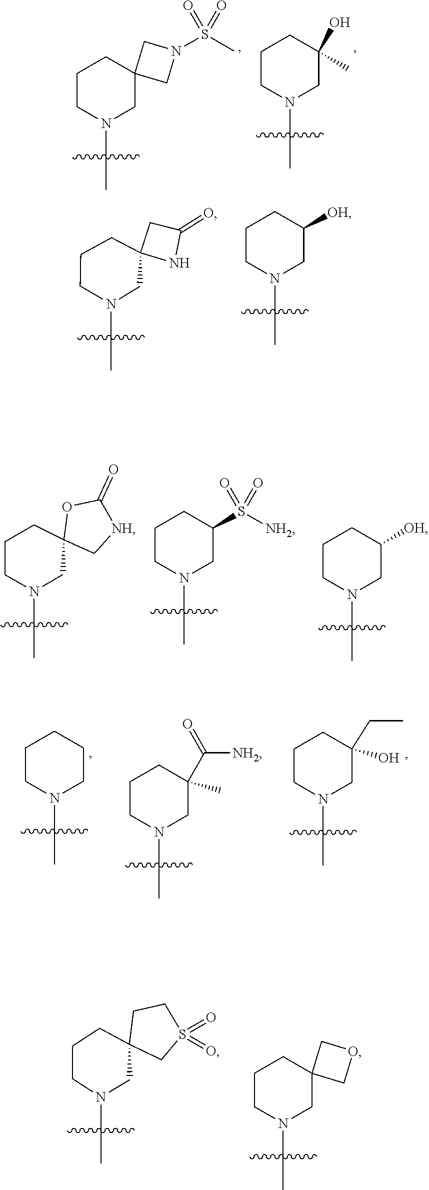

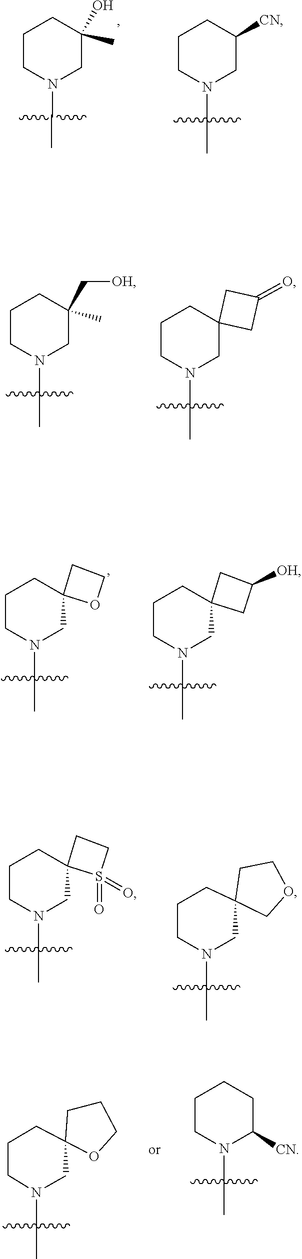

Provided herein as embodiment 115 is the compound according to embodiment 114, wherein

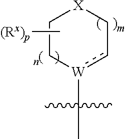

is

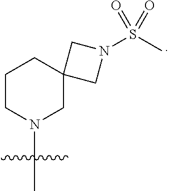 [SMILES: *N1CCCC2(C1)CN(S(C)(=O)=O)C2 ; "."]

Provided herein as embodiment 116 is the compound according to embodiment 114, wherein

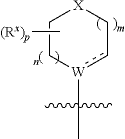

is

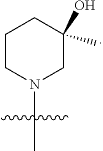 [SMILES: *N1CCC[C@@](C)(O)C1 ; "."]

Provided herein as embodiment 117 is the compound according to embodiment 114, wherein

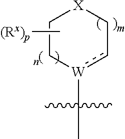

is

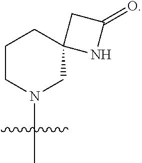 [SMILES: *N1CCC[C@]2(CC(=O)N2)C1]

Provided herein as embodiment 118 is the compound according to embodiment 114, wherein

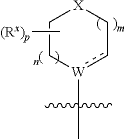

is

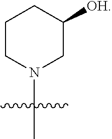 [SMILES: *N1CCC[C@@H](O)C1]

Provided herein as embodiment 119 is the compound according to embodiment 114, wherein

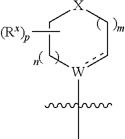

is

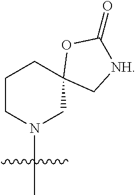 [SMILES: *N1CCC[C@@]2(CNC(=O)O2)C1]

Provided herein as embodiment 120 is the compound according to embodiment 114, wherein

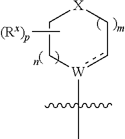

is

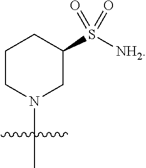 [SMILES: *N1CCC[C@@H](S(N)(=O)=O)C1]

Provided herein as embodiment 121 is the compound according to embodiment 114, wherein

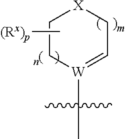

is

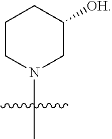 [SMILES: *N1CCC[C@H](O)C1]

Provided herein as embodiment 122 is the compound according to embodiment 114, wherein

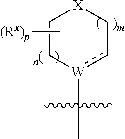

is

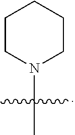 [SMILES: *N1CCCCC1 ; "."]

Provided herein as embodiment 123 is the compound according to embodiment 114, wherein

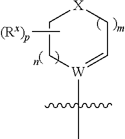

is

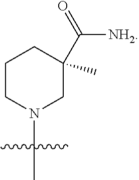 [SMILES: *N1CCC[C@@](C)(C(N)=O)C1]

Provided herein as embodiment 124 is the compound according to embodiment 114, wherein

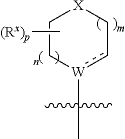

is

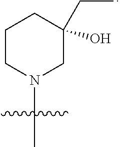 [SMILES: *N1CCC[C@@](O)(CC)C1 ; "."]

Provided herein as embodiment 125 is the compound according to embodiment 114, wherein

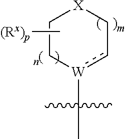

is

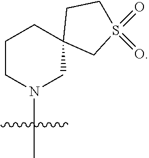 [SMILES: *N1CCC[C@]2(CCS(=O)(=O)C2)C1]

Provided herein as embodiment 126 is the compound according to embodiment 114, wherein

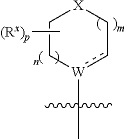

is

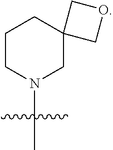 [SMILES: *N1CCCC2(COC2)C1]

Provided herein as embodiment 127 is the compound according to embodiment 114, wherein

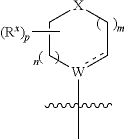

is

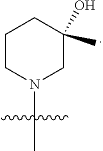 [SMILES: *N1CCC[C@](C)(O)C1 ; "."]

Provided herein as embodiment 128 is the compound according to embodiment 114, wherein

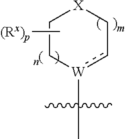

is

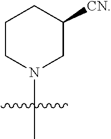 [SMILES: *N1CCC[C@@H](C#N)C1]

Provided herein as embodiment 129 is the compound according to embodiment 114, wherein

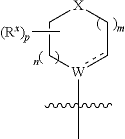

is

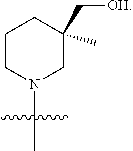 [SMILES: *N1CCC[C@@](C)(CO)C1]

Provided herein as embodiment 130 is the compound according to embodiment 114, wherein

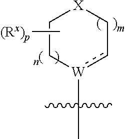

is

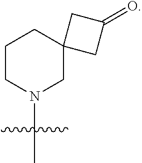 [SMILES: *N1CCCC2(CC(=O)C2)C1]

Provided herein as embodiment 131 is the compound according to embodiment 114, wherein

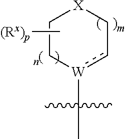

is

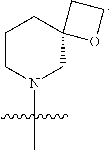 [SMILES: *N1CCC[C@]2(CCO2)C1 ; "."]

Provided herein as embodiment 132 is the compound according to embodiment 114, wherein

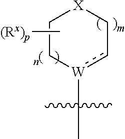

is

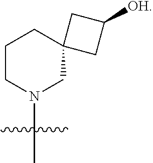 [SMILES: *N1CCC[C@]2(C1)C[C@H](O)C2]

Provided herein as embodiment 133 is the compound according to embodiment 114, wherein

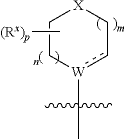

is

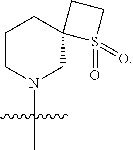 [SMILES: *N1CCC[C@]2(CCS2(=O)=O)C1]

Provided herein as embodiment 134 is the compound according to embodiment 114, wherein

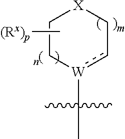

is

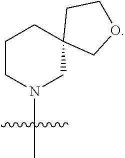 [SMILES: *N1CCC[C@]2(CCOC2)C1]

Provided herein as embodiment 135 is the compound according to embodiment 114, wherein

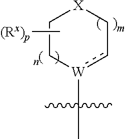

is

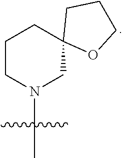 [SMILES: *N1CCC[C@]2(CCCO2)C1]

Provided herein as embodiment 136 is the compound according to embodiment 114, wherein

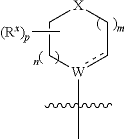

is

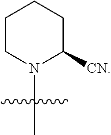 [SMILES: *N1CCCC[C@H]1C#N]

Provided herein as embodiment 137 is the compound according to embodiment 66, wherein n is 1 and m is 2 or m is 1 and n is 2. Provided herein as embodiment 138 is the compound according to embodiment 137, wherein p is 0. Provided herein as embodiment 139 is the compound according to embodiment 137, wherein p is 1. Provided herein as embodiment 140 is the compound according to embodiment 139, wherein R^{x} is halogen, cyano, oxo, hydroxyl, C₁₋₄ alkyl or —T—R^{y}. Provided herein as embodiment 141 is the compound according to embodiment 140, wherein R^{x} is fluorine, cyano, oxo, hydroxyl or —SO₂—NH₂.

Provided herein as embodiment 142 is the compound according to embodiment 137, wherein p is 2. Provided herein as embodiment 143 is the compound according to embodiment 142, wherein one R^{x} is hydroxyl and the other R^{x} is C₁₋₄ alkyl (e.g., methyl).

Provided herein as embodiment 144 is the compound according to embodiment 137, wherein p is 3. Provided herein as embodiment 145 is the compound according to embodiment 144, wherein two R^{x} are taken together with the carbon atoms to which they are attached to form a bridged ring wherein the bridge atoms are —C₁₋₄ alkylene and the remaining R^{x} is hydroxyl. Provided herein as embodiment 146 is the compound according to embodiment 145, wherein two R^{x} are taken together with the carbon atoms to which they are attached to form a methylene bridged ring and the remaining R^{x} is hydroxyl.

Provided herein as embodiment 147 is the compound according to embodiment 66, wherein n is 2 and m is 2. Provided herein as embodiment 148 is the compound according to embodiment 147, wherein p is 0.

Provided herein as embodiment 149 is the compound according to any one of embodiments 137-148, wherein

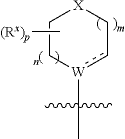

is

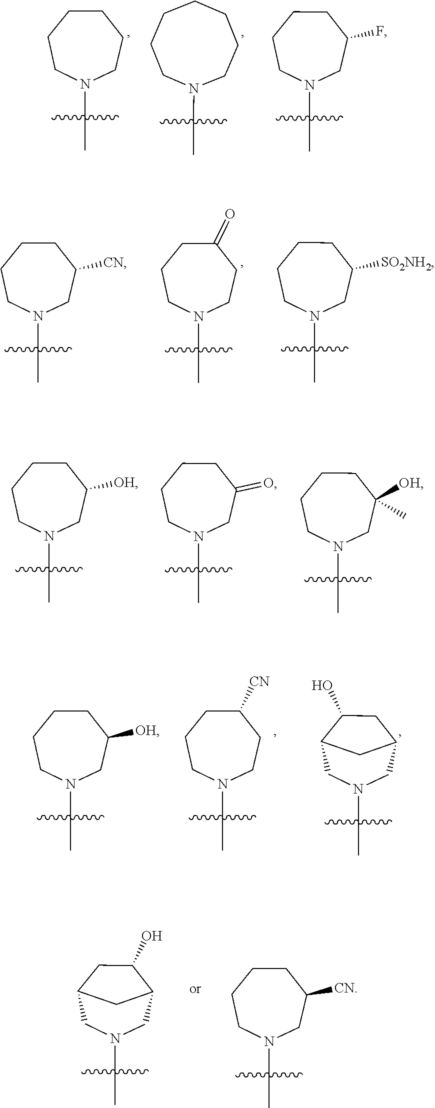

Provided herein as embodiment 150 is the compound according to embodiment 149, wherein

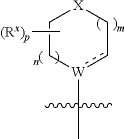

is

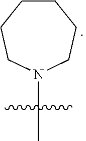 [SMILES: *N1CCCCCC1]

Provided herein as embodiment 151 is the compound according to embodiment 149, wherein

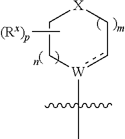

is

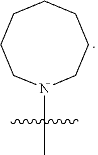 [SMILES: *N1CCCCCCC1]

Provided herein as embodiment 152 is the
compound according to embodiment 149, wherein

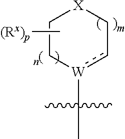

is

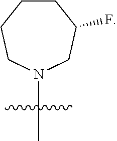 [SMILES: *N1CCCC[C@H](F)C1]

Provided herein as embodiment 153 is the compound according to embodiment 149, wherein

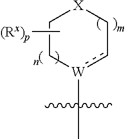

is

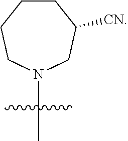 [SMILES: *N1CCCC[C@H](C#N)C1]

Provided herein as embodiment 154 is the compound according to embodiment 149, wherein

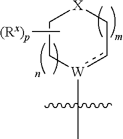

is

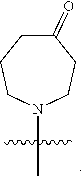 [SMILES: *N1CCCC(=O)CC1]

Provided herein as embodiment 155 is the compound according to embodiment 149, wherein

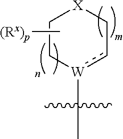

is

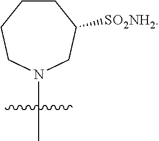 [SMILES: *N1CCCC[C@H](S(N)(=O)=O)C1]

Provided herein as embodiment 156 is the compound according to embodiment 149, wherein

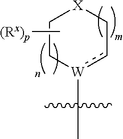

is

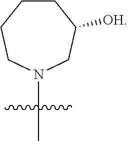 [SMILES: *N1CCCC[C@H](O)C1]

Provided herein as embodiment 157 is the compound according to embodiment 149, wherein

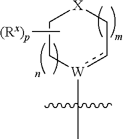

is

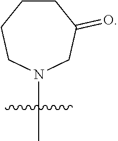 [SMILES: *N1CCCCC(=O)C1]

Provided herein as embodiment 158 is the compound according to embodiment 149, wherein

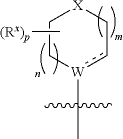

is

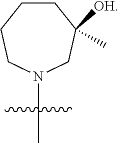 [SMILES: *N1CCCC[C@@](C)(O)C1]

Provided herein as embodiment 159 is the compound according to embodiment 149, wherein

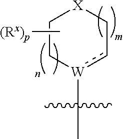

is

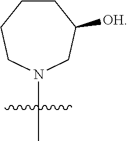 [SMILES: *N1CCCC[C@@H](O)C1]

Provided herein as embodiment 160 is the compound according to embodiment 149, wherein

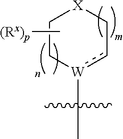

is

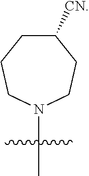 [SMILES: *N1CCC[C@H](C#N)CC1]

Provided herein as embodiment 161 is the compound according to embodiment 149, wherein

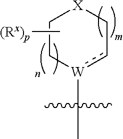

is

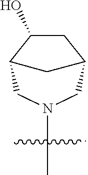 [SMILES: *N1C[C@@H]2C[C@H](C1)[C@H](O)C2]

Provided herein as embodiment 162 is the compound according to embodiment 149, wherein

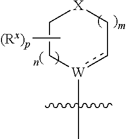

is

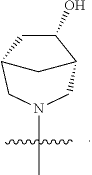 [SMILES: *N1C[C@H]2C[C@@H](C1)[C@@H](O)C2]

Provided herein as embodiment 163 is the compound according to embodiment 149, wherein

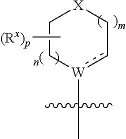

is

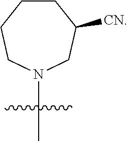 [SMILES: *N1CCCC[C@@H](C#N)C1]

Provided herein as embodiment 164 is the compound according to embodiment 26, wherein X is —CH═CH—. Provided herein as embodiment 165 is the compound according to embodiment 164, wherein n is 1 and m is 1. Provided herein as embodiment 166 is the compound according to embodiment 165, wherein p is 1. Provided herein as embodiment 167 is the compound according to embodiment 166, wherein R^{x} is hydroxyl.

Provided herein as embodiment 168 is the compound according to any one of embodiments 164-167, wherein

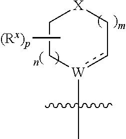

is

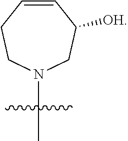 [SMILES: *N1CCC=C[C@H](O)C1]

Provided herein as embodiment 169 is the compound according to any one of embodiments 1-2, wherein W is —C═. Provided herein as embodiment 170 is the compound according to embodiment 169, wherein X is O. Provided herein as embodiment 171 is the compound according to embodiment 170, wherein [symbol] is a double bond. Provided herein as embodiment 172 is the compound according to embodiment 171, wherein n is 0 and m is 2. Provided herein as embodiment 173 is the compound according to embodiment 172, wherein p is 0.

Provided herein as embodiment 174 is the compound according to any one of embodiments 169-173, wherein

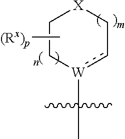

is

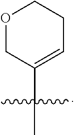 [SMILES: *C1=CCCOC1 ; "."]

Provided herein as embodiment 175 is the compound according to any one of embodiments 1-2, wherein W is —CH—. Provided herein as embodiment 176 is the compound according to embodiment 175, wherein X is a bond. Provided herein as embodiment 177 is the compound according to embodiment 176, wherein [symbol] is a single bond. Provided herein as embodiment 178 is the compound according to embodiment 177, wherein n is 0 and m is 0. Provided herein as embodiment 179 is the compound according to embodiment 178, wherein p is 0.

Provided herein as embodiment 180 is the compound according to any one of embodiments 175-180, wherein

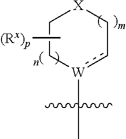

is

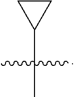 [SMILES: *C1CC1]

Provided herein as embodiment 181 is the compound according to any one of embodiments 1-180, wherein R⁸ is amino and R⁹ is cyano.

Provided herein as embodiment 182 is the compound according to any one of embodiments 1-181, wherein R² is halogen, C₁₋₄ alkyl, C₂₋₄ alkenyl or cyano. Provided herein as embodiment 183 is the compound according to embodiment 182, wherein R² is chlorine, methyl, ethyl, vinyl or cyano. Provided herein as embodiment 184 is the compound according to embodiment 182, wherein R² is chlorine. Provided herein as embodiment 185 is the compound according to embodiment 182, wherein R² is methyl or ethyl. Provided herein as embodiment 186 is the compound according to embodiment 182, wherein R² is methyl. Provided herein as embodiment 187 is the compound according to embodiment 182, wherein R² is ethyl. Provided herein as embodiment 188 is the compound according to embodiment 182, wherein R² is vinyl (i.e., 2-ethenyl). Provided herein as embodiment 189 is the compound according to embodiment 182, wherein R² is cyano.

Provided herein as embodiment 190 is the compound according to any one of embodiments 1-189, wherein R⁴ is halogen (e.g., fluorine).

Provided herein as embodiment 191 is the compound according to any one of embodiments 1-190, wherein R⁴ is fluorine.

Provided herein as embodiment 192 is the compound according to any one of embodiments 1-191, wherein R³ is hydrogen or halogen (e.g., fluorine). Provided herein as embodiment 193 is the compound according to embodiment 192, wherein R³ is hydrogen. Provided herein as embodiment 194 is the compound according to embodiment 192, wherein R³ is fluorine.

Provided herein as embodiment 195 is the compound according to any one of 1-194, wherein R^{q} is hydrogen. Provided herein as embodiment 196 is the compound according to any one of embodiments 1-194, wherein R^{q} is halogen (e.g., chlorine or fluorine). Provided herein as embodiment 197 is the compound according to any one of embodiments 1-194, wherein R^{q} is C₁₋₄ alkyl (e.g., methyl).

Provided herein as embodiment 198 is the compound according to any one of embodiments 1-197, wherein R^{q} is attached as illustrated in Formula (IIa):

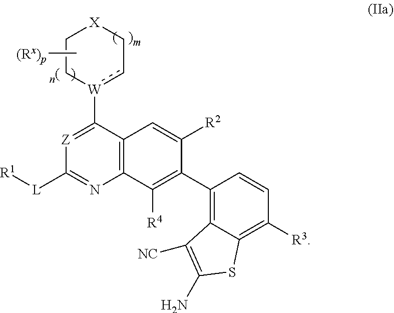

Provided herein as embodiment 199 is the compound according to any one of embodiments 1-198, wherein R^{q} is attached as illustrated in Formula (IIb):

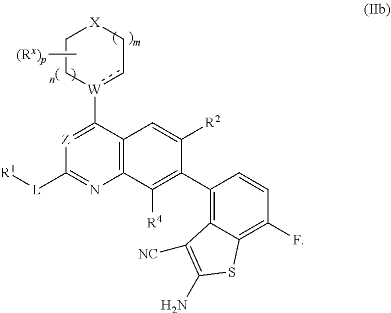

Provided herein as embodiment 200 is the compound according to any one of embodiments 1-199, wherein R^{q} is attached as illustrated in Formula (IIc):

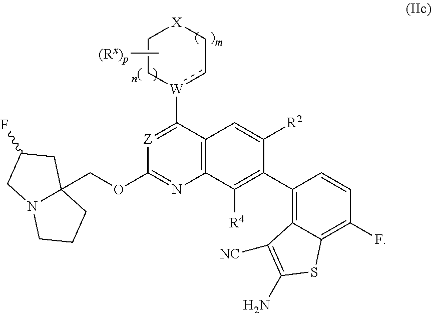

Provided herein as embodiment 201 is the compound according to any one of embodiments 1-200, wherein R^{q} is attached as illustrated in Formula (IId):

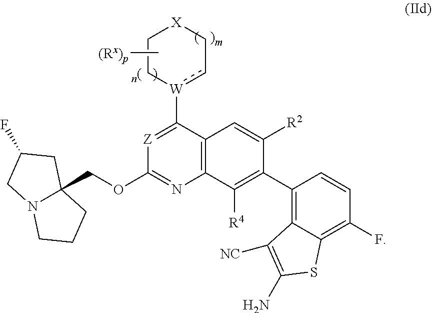

Provided herein as embodiment 201 is the compound according to embodiment 1, wherein the compound is selected from one of the following compounds:
2-Amino-4-(6-chloro-8-fluoro-2-(((2R,7aS)-2-fluorotetrahydro-1H-pyrrolizin-7a(5H)-yl)methoxy)-4-(1,5-oxazocan-5-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile;
2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-4-(6-ethyl-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((R)-3-hydroxyazepan-1-yl)quinazolin-7-yl)-7-fluorobenzo[b]thiophene-3-carbonitrile;
2-Amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile;
2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-3-carbonitrile;
2-Amino-4-(6-chloro-8-fluoro-4-(6-fluoro-1,4-oxazepan-4-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(4-oxoazepan-1-yl)quinazolin-7-yl)-7-fluorobenzo[b]thiophene-3-carbonitrile;
2-Amino-4-(6-chloro-8-fluoro-4-(6-fluoro-1,4-oxazepan-4-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((S)-6-hydroxy-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-hydroxy-2,3,6,7-tetrahydro-1H-azepin-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(1,4-oxazocan-4-yl)quinazolin-7-yl)-7-fluorobenzo[b]thiophene-3-carbonitrile;
2-Amino-4-(6-chloro-4-(6,6-difluoro-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((S)-3-hydroxyazepan-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-hydroxy-3-methylazepan-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile;
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-4-carbonitrile;
(2S)-1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)pyrrolidine-2-carbonitrile;
2-Amino-4-(6-chloro-8-fluoro-2-(((2R,7aS)-2-fluorotetrabydro-1H-pyrrolizin-7a(5H)-yl)methoxy)-4-(6-oxo-1,4-oxazepan-4-yl)quinazolin-7-yl)-7-fluorobenzo[b]thiophene-3-carbonitrile;
2-Amino-4-(6-chloro-4-(6-(difluoromethyl)-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile; or
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile.

Provided herein as embodiment 202 is the compound according to embodiment 1, wherein the compound is selected from one of the following compounds:
2-Amino-4-(6-chloro-8-fluoro-2-(((2R,7aS)-2-fluorotetrahydro-1H-pyrrolizin-7a(5H)-yl)methoxy)-4-(1,5-oxazocan-5-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile;
2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-4-(6-ethyl-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((R)-3-hydroxyazepan-1-yl)quinazolin-7-yl)-7-fluorobenzo[b]thiophene-3-carbonitrile;
2-Amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile;
2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile; or
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-3-carbonitrile.

Provided herein as embodiment 203 is the compound according to embodiment 1, wherein the compound is selected from one of the following compounds:
2-Amino-4-(6-chloro-8-fluoro-2-(((2R,7aS)-2-fluorotetrahydro-1H-pyrrolizin-7a(5H)-yl)methoxy)-4-(1,5-oxazocan-5-yl)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile;
2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-4-(6-ethyl-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-((R)-3-hydroxyazepan-1-yl)quinazolin-7-yl)-7-fluorobenzo[b]thiophene-3-carbonitrile;
2-Amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile;
2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-3-carbonitrile;
2-Amino-4-(6-chloro-8-fluoro-4-(6-fluoro-1,4-oxazepan-4-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Isomer 2);
2-Amino-4-(6-chloro-8-fluoro-2-(((2R,7aS)-2-fluorotetrahydro-1H-pyrrolizin-7a(5H)-yl)methoxy)-4-(4-oxoazepan-1-yl)quinazolin-7-yl)-7-fluorobenzo[b]thiophene-3-carbonitrile;
2-Amino-4-(6-chloro-8-fluoro-4-(6-fluoro-1,4-oxazepan-4-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-((S)-6-hydroxy-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-hydroxy-2,3,6, 7-tetrahydro-1H-azepin-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(1,4-oxazocan-4-yl)quinazolin-7-yl)-7-fluorobenzo[b]thiophene-3-carbonitrile;
2-Amino-4-(6-chloro-4-(6,6-difluoro-1,4-oxazepan-4-yl)-8-fluoro-2-(((2R,7aS)-2-fluorotetrahydro-1H-pyrrolizin-7a (H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((S)-3-hydroxyazepan-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-hydroxy-3-methylazepan-1-yl)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile;
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-4-carbonitrile;
(2S)-1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)pyrrolidine-2-carbonitrile;
2-Amino-4-(6-chloro-8-fluoro-2-(((2R,7aS)-2-fluorotetrahydro-1H-pyrrolizin-7a(5H)-yl)methoxy)-4-(6-oxo-1,4-oxazepan-4-yl)quinazolin-7-yl)-7-fluorobenzo[b]thiophene-3-carbonitrile;
2-Amino-4-(6-chloro-4-(6-(difluoromethyl)-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Isomer 1); or
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile.

Provided herein as embodiment 204 is the compound according to embodiment 1, wherein the compound is selected from one of the following compounds;
2-Amino-4-(6-chloro-8-fluoro-2-(((2R,7aS)-2-fluorotetrahydro-1H-pyrrolizin-7a(5H)-yl)methoxy)-4-(1,5-oxazocan-5-yl)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile;
2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiopbene-3-carbonitrile;
2-Amino-4-(6-chloro-4-(6-ethyl-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Isomer 1);
2-Amino-4-(6-chloro-8-fluoro-2-(((2R,7aS)-2-fluorotetrahydro-1H-pyrrolizin-7a(5H)-yl)methoxy)-4-((R)-3-hydroxyazepan-1-yl)quinazolin-7-yl)-7-fluorobenzo[b]thiopbene-3-carbonitrile;
2-Amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile;
2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile; or
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-3-carbonitrile.

The foregoing merely summarizes certain aspects of this disclosure and is not intended, nor should it be construed, as limiting the disclosure in any way.

### Formulation, and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker, New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as embodiment 205 is a pharmaceutical composition comprising the compound according to any one of embodiments 1-204, or a tautomer thereof, or a pharmaceutically acceptable salt of said compound or said tautomer, and a pharmaceutically acceptable excipient.

Provided herein as embodiment 206 is a compound according to any one of embodiments 1-204, or a tautomer thereof, or a pharmaceutically acceptable salt of said compound or said tautomer, or the pharmaceutical composition according to embodiment 205 for use as a medicament.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In one embodiment, the disclosure provides methods of using the compounds or pharmaceutical compositions of the present disclosure to treat disease conditions, including but not limited to conditions implicated by KRAS G12D, G12V, G12A, G12S or G12C mutation (e.g., cancer). The cancer types are non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

KRAS G12D mutations occur with the alteration frequencies shown in the table below (TCGA data sets;¹⁻³ For example, the table shows that 32.4% of subjects with pancreatic cancer have a cancer wherein one or more cells express KRAS G12D mutant protein. Accordingly, the compounds provided herein, which bind to KRAS^{G12D} (see Section entitled “Biological Evaluation” below) are useful for treatment of subjects having a cancer, including, but not limited to the cancers listed in the table below.

| | Alteration |
|---|---|
| Cancer Type | Frequency |
| Pancreatic Adenocarcinoma (PAAD) | 32.4 |
| Colon Adenocarcinoma (COAD) | 12.25 |
| Rectal adenocarcinoma (READ) | 8.03 |
| Uterine corpus endometrial carcinoma | 6.04 |
| (UCEC) | |
| Lung Adenocarcinoma (LUAD) | 3.53 |
| Plasma Cell Tumors | 2.92 |
| Stomach Adenocarcinoma (STAD) | 2.27 |
| Bladder urothelial carcinoma (BLCA) | 1.46 |
| Cervical Squamous carcinoma (CESC) | 1.38 |
| Kidney Adenocarcinoma | 1.07 |
| Thymic Cancer | 0.81 |
| Myeloid Leukemia (LAML) | 0.69 |
| Liver Hepatocellular Carcinoma (LIHC) | 0.55 |
| Glioblastoma multiforme (GBM) | 0.51 |
| Skin Cutaneous Melanoma (SKCM) | 0.43 |
| Bladder Cancer | 0.4 |
| Prostate Adenocarcinoma (PRAD) | 0.2 |
| Breast Invasive Carcinoma (BRCA) | 0.1 |

Provided herein as embodiment 207 is a compound according to any one of embodiments 1-204 or a pharmaceutically acceptable salt thereof, or the pharmaceutical composition according to embodiment 205 for use in treating cancer.

Provided herein as Embodiment 208 is a compound according to any one of Embodiments 1-204 or a pharmaceutically acceptable salt thereof, or the pharmaceutical composition according to Embodiment 205 for use in treating cancer, wherein one or more cells express KRAS G12D, G12V, G12A, G12S or G12C mutant protein.

Provided herein as Embodiment 209 is the compound or pharmaceutical composition for use of Embodiment 207 or 208, wherein the cancer is pancreatic cancer, colorectal cancer, non-small cell lung cancer, small bowel cancer, appendiceal cancer, cancer of unknown primary, endometrial cancer, mixed cancer types, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 210 is a use of the compound according to any one of Embodiments 1-204 or a pharmaceutically acceptable salt thereof, or the pharmaceutical composition according to Embodiment 205 in the preparation of a medicament for treating cancer.

Provided herein as Embodiment 211 is a use of the compound according to any one of Embodiments 1-204 or a pharmaceutically acceptable salt thereof, or the pharmaceutical composition according to Embodiment 205 in the preparation of a medicament for treating cancer, wherein one or more cells express KRAS G12D, G12V, G12A, G12S or G12C mutant protein.

Provided herein as Embodiment 212 is the use according to Embodiment 210 or 211, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 213 is a method of treating cancer in a subject in need thereof, the method comprising administering to the subject a therapeutically effective amount of the compound according to any one of to any one of Embodiments 1-204 or a pharmaceutically acceptable salt thereof.

Provided herein as Embodiment 214 is a method of treating cancer in a subject in need thereof, the method comprising administering to the subject a therapeutically effective amount of the compound according to any one of to any one of Embodiments 1-204 or a pharmaceutically acceptable salt thereof, wherein one or more cells express KRAS G12D, G12V, G12A, G12S or G12C mutant protein.

Provided herein as Embodiment 215 is the method according to Embodiment 213 or 214, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 216 is the method according to Embodiment 213 or 214, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

Provided herein as Embodiment 217 is the method according to Embodiment 216, wherein the cancer is non-small cell lung cancer.

Provided herein as Embodiment 218 is the method according to Embodiment 216, wherein the cancer is colorectal cancer.

Provided herein as Embodiment 219 is the method according to Embodiment 216, wherein the cancer is pancreatic cancer.

Provided herein as Embodiment 220 is the method according to anyone of Embodiments 213-219, wherein the subject has a cancer that was determined to have one or more cells expressing the KRAS G12D, G12V, G12A, G12S or G12C mutant protein prior to administration of the compound or a pharmaceutically acceptable salt thereof.

### Combination Therapy

The present disclosure also provides methods for combination therapies in which an agent known to modulate other pathways, or other components of the same pathway, or even overlapping sets of target enzymes are used in combination with a compound of the present disclosure or a pharmaceutically acceptable salt thereof. In one aspect, such therapy includes but is not limited to the combination of one or more compounds of the disclosure with chemotherapeutic agents, therapeutic antibodies, and radiation treatment, to provide a synergistic or additive therapeutic effect. See, e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

Provided herein as Embodiment 198 is the method according to anyone of Embodiments 190-197, which further comprises simultaneous, separate, or sequential administration of an effective amount of a second compound, wherein the second compound is an Aurora kinase A inhibitor, AKT inhibitor, arginase inhibitor, CDK4/6 inhibitor, ErbB family inhibitor, ERK inhibitor, FAK inhibitor, FGFR inhibitor, glutaminase inhibitor, IGF-IR inhibitor, KIF18A inhibitor, MCL-1 inhibitor, MEK inhibitor, mTOR inhibitor, PD-1 inhibitor, PD-L1 inhibitor, PI3K inhibitor, Raf kinase inhibitor, SHP2 inhibitor, SOS1 inhibitor, Src kinase inhibitor, or one or more chemotherapeutic agent.

In one embodiment, the second compound is administered as a pharmaceutically acceptable salt. In another embodiment the second compound is administered as a pharmaceutical composition comprising the second compound or a pharmaceutically acceptable salt thereof and a pharmaceutically acceptable excipient.

### Aurora Kinase A Inhibitors

Provided herein is the method according to anyone of Embodiments 213-220, which further comprises simultaneous, separate, or sequential administration of an effective amount of a second compound, wherein the second compound is an Aurora kinase A inhibitor.

Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03,7]tetradeca-1(14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7] undeca-2 (7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino) pyrimidin-4-yl]-1,3-thiazol-2-amine), TAS-119, BI 811283, and TTP607.

### AKT Inhibitors

Provided herein is the method according to anyone of Embodiments 213-220, which further comprises simultaneous, separate, or sequential administration of an effective amount of a second compound, wherein the second compound is an AKT inhibitor.

Exemplary AKT inhibitors for use in the methods provided herein include, but are not limited to, afuresertib, capivasertib, ipatasertib, uprosertib, BAY1125976 (2-[4-(1-aminocyclobutyl)phenyl]-3-phenylimidazo[1,2-b]pyridazine-6-carboxamide), ARQ 092 (3-[3-[4-(1-aminocyclobutyl)phenyl]-5-phenylimidazo[4,5-b]pyridin-2-yl]pyridin-2-amine), MK2206 (8-[4-(1-aminocyclobutyl)phenyl]-9-phenyl-2H-[1,2,4]triazolo[3,4-f][1,6]naphthyridin-3-one), SR13668 (indolo [2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1(9),5-dien-8-one), ARQ 751 (N-(3-aminopropyl)-N-[(1R)-1-(3-anilino-7-chloro-4-oxoquinazolin-2-yl) but-3-ynyl]-3-chloro-2-fluorobenzamide), RX-0201, and LY2780301.

### Arginase Inhibitors

Provided herein is the method according to anyone of Embodiments 213-220, which further comprises simultaneous, separate, or sequential administration of an effective amount of a second compound, wherein the second compound is an arginase inhibitor.

Exemplary arginase inhibitors for use in the methods provided herein include, but are not limited to, numidargistat and CB 280.

### CDK4/6 Inhibitors

Provided herein is the method according to anyone of Embodiments 213-220, which further comprises simultaneous, separate, or sequential administration of an effective amount of a second compound, wherein the second compound is a CDK4/6 inhibitor.

The term “CDK 4/6” as used herein refers to cyclin dependent kinases (“CDK”) 4 and 6, which are members of the mammalian serine/threonine protein kinases.

The term “CDK 4/6 inhibitor” as used herein refers to a compound that is capable of negatively modulating or inhibiting all or a portion of the enzymatic activity of CDK 4 and/or 6.

Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7 (8H)-one, 6-(difluoromethyl)-8-[(1R,2R)-2-hydroxy-2-methylcyclopentyl]-2-[[1-(methylsulfonyl)-4-piperidinyl]amino]).

In one embodiment, the CDK4/6 inhibitor is palbociclib.

### ErbB Family Inhibitors

Provided herein is the method according to anyone of Embodiments 213-220, which further comprises simultaneous, separate, or sequential administration of an effective amount of a second compound, wherein the second compound is an ErbB family inhibitor.

The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4).

The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members.

In one embodiment, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In one embodiment, the anti-EGFR antibody is cetuximab. In one embodiment, the anti-EGFR antibody is panitumumab.

In another embodiment the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine.

In yet another embodiment the ErbB family inhibitor is a HER3 inhibitor, e.g., an anti-HER3 antibody, such as HMBD-001 (Hummingbird Bioscience).

In one embodiment, the ErbB family inhibitor is a combination of an anti-EGFR antibody and anti-HER2 antibody.

In one embodiment, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino) but-2-enamide).

In one embodiment, the irreversible ErbB family inhibitor is afatinib. In one embodiment, the irreversible ErbB family inhibitor is dacomitinib.

In one embodiment, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide), AEE788 ((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine).

In one embodiment, the reversible ErbB family inhibitor is sapitinib. In one embodiment, the reversible ErbB family inhibitor is tarloxotinib.

### ERK Inhibitors

Provided herein is the method according to anyone of Embodiments 213-220, which further comprises simultaneous, separate, or sequential administration of an effective amount of a second compound, wherein the second compound is an ERK inhibitor.

Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-metbylphenyl]prop-2-enamide), LY3214996 (6,6-dimethyl-2-[2-[(2-methylpyrazol-3-yl)amino]pyrimidin-4-yl]-5-(2-morpholin-4-ylethyl)thieno[2,3-c]pyrrol-4-one), KO-947 (1,5,6,8-tetrahydro-6-(phenylmethyl)-3-(4-pyridinyl)-7H-pyrazolo[4,3-g] quinazolin-7-one), ASTX029, LTT462, and JSI-1187.

### FAK Inhibitors

Provided herein is the method according to anyone of Embodiments 213-220, which further comprises simultaneous, separate, or sequential administration of an effective amount of a second compound, wherein the second compound is a FAK inhibitor.

Exemplary FAK inhibitors for use in the methods provided herein include, but are not limited to, GSK2256098 (2-[[5-chloro-2-[(5-methyl-2-propan-2-ylpyrazol-3-yl)amino]pyridin-4-yl]amino]-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[[2-(2-methoxy-4-morpholin-4-ylanilino)-5-(trifluoromethyl)pyridin-4-yl]amino]-N-methylbenzamide), and APG-2449.

### FGFR Inhibitors

Provided herein is the method according to anyone of Embodiments 213-220, which further comprises simultaneous, separate, or sequential administration of an effective amount of a second compound, wherein the second compound is an FGFR inhibitor.

Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), Debio 1347 ([5-amino-1-(2-methyl-3H-benzimidazol-5-yl)pyrazol-4-yl]-(1H-indol-2-yl) methanone), INCB062079, H3B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

### Glutaminase Inhibitors

Provided herein is the method according to anyone of Embodiments 213-220, which further comprises simultaneous, separate, or sequential administration of an effective amount of a second compound, wherein the second compound is a glutaminase inhibitor.

Exemplary glutaminase inhibitors for use in the methods provided herein include, but are not limited to, telaglenastat, IPN60090, and OP 330.

### IGF-1R Inhibitors

Provided herein is the method according to anyone of Embodiments 213-220, which further comprises simultaneous, separate, or sequential administration of an effective amount of a second compound, wherein the second compound is an IGF-IR inhibitor.

Exemplary IGF-IR inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][1,2,4]triazin-2-yl]-N-(6-fluoropyridin-3-yl)-2-methylpyrrolidine-2-carboxamide), KW-2450 (N-[5-[[4-(2-hydroxyacetyl) piperazin-1-yl]methyl]-2-[(E)-2-(1H-indazol-3-yl)ethenyl]phenyl]-3-methylthiophene-2-carboxamide), PL225B, AVE1642, and BIIB022.

### KIF18A Inhibitors

Provided herein is the method according to anyone of Embodiments 213-220, which further comprises simultaneous, separate, or sequential administration of an effective amount of a second compound, wherein the second compound is a KIF18A inhibitor.

Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety.

### MCL-1 Inhibitors

Provided herein is the method according to anyone of Embodiments 213-220, which further comprises simultaneous, separate, or sequential administration of an effective amount of a second compound, wherein the second compound is an MCL-1 inhibitor.

Exemplary MEK inhibitors for use in the methods provided herein include, but are not limited to, murizatoclax, tapotoclax, AZD 5991 ((3aR)-5-chloro-2,11,12,24,27,29-hexahydro-2,3,24,33-tetramethyl-22H-9,4,8-(metheniminomethyno)-14,20:26,23-dimetheno-10H,20H-pyrazolo[4,3-1][2,15,22,18,19]benzoxadithiadiazacyclohexacosine-32-carboxylic acid), MIK 665 ((αR)-α-[[(5S)-5-[3-Chloro-2-methyl-4-[2-(4-methyl-1-piperazinyl)ethoxy]phenyl]-6-(4-fluorophenyl)thieno[2,3-d]pyrimidin-4-yl]oxy]-2-[[2-(2-methoxyphenyl)-4-pyrimidinyl]methoxy]benzenepropanoic acid), and ABBV-467.

In one embodiment, the MCL-1 inhibitor is murizatoclax. In another embodiment, the MCL-1 inhibitor is tapotoclax.

### MEK Inhibitors

Provided herein is the method according to anyone of Embodiments 213-220, which further comprises simultaneous, separate, or sequential administration of an effective amount of a second compound, wherein the second compound is MEK inhibitor.

Exemplary MEK inhibitors for use in the methods provided herein include, but are not limited to, trametinib, cobimetinib, selumetinib, pimasertib, refametinib, PD-325901 (N-[(2R)-2,3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy) imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), CI-1040 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554.

In one embodiment, the MEK inhibitor is trametinib.

mTOR Inhibitors

Provided herein is the method according to anyone of Embodiments 213-220, which further comprises simultaneous, separate, or sequential administration of an effective amount of a second compound, wherein the second compound is an mTOR inhibitor.

Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib, Torin-1 (1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine).

In one embodiment, the mTOR inhibitor is everolimus.

### PD-1 Inhibitors

Provided herein is the method according to anyone of Embodiments 213-220, which further comprises simultaneous, separate, or sequential administration of an effective amount of a second compound, wherein the second compound is a PD-1 inhibitor.

Exemplary PD-1 inhibitors for use in the methods provided herein include, but are not limited to, pembrolizumab, nivolumab, cemiplimab, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference.

In one embodiment, the PD-1 inhibitor is pembrolizumab. In another embodiment the PD-1 inhibitor is the Anti-PD-1 Antibody A.

### PD-L1 Inhibitors

Provided herein is the method according to anyone of Embodiments 213-220, which further comprises simultaneous, separate, or sequential administration of an effective amount of a second compound, wherein the second compound is a PD-L1 inhibitor.

Exemplary PD-L1 inhibitors for use in the methods provided herein include, but are not limited to, atczolizumab, avelumab, durvalumab, ZKAB001, TG-1501, SHR-1316, MSB2311, MDX-1105, KN035, IMC-001, HLX20, FAZ053, CS1001, CK-301, CBT-502, BGB-A333, BCD-135, and A167.

In one embodiment, the PD-L1 inhibitor is atezolizumab.

### PI3K Inhibitors

Provided herein is the method according to anyone of Embodiments 213-220, which further comprises simultaneous, separate, or sequential administration of an effective amount of a second compound, wherein the second compound is a PI3K inhibitor.

Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide), ME-401 (N-[2-methyl-1-[2-(1-methylpiperidin-4-yl)phenyl] propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl)ethynyl]-1-oxo-2-phenylisoquinolin-3-yl]ethyl]pyrazolo[1,5-a]pyrimidine-3-carboxamide), SF1126 ((2S)-2-[[(2S)-3-carboxy-2-[[2-[[(2S)-5-(diaminomethylidencamino)-2-[[4-oxo-4-[[4-(4-oxo-8-phenylchromen-2-yl) morpholin-4-ium-4-yl]methoxy]butanoyl]amino]pentanoyl]amino]acetyl]amino]propanoyl]amino]-3-hydroxypropanoate), XL147 (N-[3-(2,1,3-benzothiadiazol-5-ylamino) quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

### Raf Kinase Inhibitors

Provided herein is the method according to anyone of Embodiments 213-220, which further comprises simultaneous, separate, or sequential administration of an effective amount of a second compound, wherein the second compound is a Raf kinase inhibitor.

The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers.

The term “Raf kinase inhibitor” as used herein refers to a compound that is capable of negatively modulating or inhibiting all or a portion of the enzymatic activity of one or more member of the Raf family kinases, or is capable of disrupting Raf homodimer or heterodimer formation to inhibit activity.

In one embodiment, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5,-morpholino-6′-((tetrahydro-2H-pyran-4-yl)oxy)-[3,3′-bipyridin]-5-yl)-3-(trifluoromethyl) benzamide), LXH254 (N-(3-(2-(2-hydroxyethoxy)-6-morpholinopyridin-4-yl)-4-methylphenyl)-2-(trifluoromethyl) isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl) urea), CCT196969 (1-(3-(tert-butyl)-1-phenyl-1H-pyrazol-5-yl)-3-(2-fluoro-4-(3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide).

In one embodiment, the Raf kinase inhibitor is encorafenib. In one embodiment, the Raf kinase inhibitor is sorafenib. In one embodiment, the Raf kinase inhibitor is lifirafenib.

### SHP2 Inhibitors

Provided herein is the method according to anyone of Embodiments 213-220, which further comprises simultaneous, separate, or sequential administration of an effective amount of a second compound, wherein the second compound is a SHP2 inhibitor.

Exemplary SHP2 inhibitors for use in the methods provided herein include, but are not limited to, SHP-099 (6-(4-amino-4-methylpiperidin-1-yl)-3-(2,3-dichlorophenyl)pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine). In one embodiment, the SHP inhibitor for use in the methods provided herein is RMC-4630 (Revolution Medicine).

In another embodiment, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azctidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 2172652-48-9).

In another embodiment, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio] pyrazolo[1,5-a]pyrazin-4-yl]-3-methyl-2-oxa-8-azaspiro[4.5]decan-4-amine (CAS 2240982-57-2), 4-[(38,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (38,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6).

In one embodiment, the SHP inhibitor for use in the methods provided herein is (IR)-8-[5-(2,3-dichlorophenyl)-6-methylimidazo[1,5-a]pyrazin-8-yl]-8-azaspiro[4.5]decan-1-amine (CAS 2240981-78-4).

In another embodiment, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(IR)-1-amino-8-azaspiro[4.5]dec-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thio]-3-[(38,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(38,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol.

In one embodiment, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5).

In one embodiment, the SHP2 inhibitor for use in the methods provided herein is an inhibitor disclosed in U.S. Pat. No. 10,590,090 B2, US 2020/017517 A1, US 2020/017511 A1, or WO 2019/075265 A1, each of which is herewith incorporated by reference in its entirety.

### SOS1 Inhibitors

Provided herein is the method according to anyone of Embodiments 213-220, which further comprises simultaneous, separate, or sequential administration of an effective amount of a second compound, wherein the second compound is an SOS1 inhibitor.

Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxyquinazolin-4-amine), and BI 1701963.

### Src Kinase Inhibitors

Provided herein is the method according to anyone of Embodiments 213-220, which further comprises simultaneous, separate, or sequential administration of an effective amount of a second compound, wherein the second compound is a Sre kinase inhibitor.

The term “Src kinase” as used herein refers to a member of a mammalian nonreceptor tyrosine kinase family including: Src, Yes, Fyn, and Fgr (SrcA subfamily); Lck, Hck, Blk, and Lyn (SrcB subfamily), and Frk subfamily.

The term “Src kinase inhibitor” as used herein refers to a compound that is capable of negatively modulating or inhibiting all or a portion of the enzymatic activity of one or more member of the Src kinases.

Exemplary Src kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl) acetamide), SU6656 ((Z)-N,N-dimethyl-2-oxo-3-((4,5,6,7-tetrahydro-1H-indol-2-yl)methylene) indoline-5-sulfonamide), PP 1 (1-(tert-butyl)-3-(p-tolyl)-1H-pyrazolo[3,4-d]pyrimidin-4-amine), WH-4-023 (2,6-dimethylphenyl (2,4-dimethoxyphenyl) (2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl) acetamide).

In one embodiment, the Sre kinase inhibitor is dasatinib. In one embodiment, the Src kinase inhibitor is saracatinib. In one embodiment, the Sre kinase inhibitor is ponatinib. In one embodiment, the Src kinase inhibitor is vandetanib. In one embodiment, the Sre kinase inhibitor is KX-01.

### Chemotherapeutic Agents

Provided herein is the method according to anyone of Embodiments 213-220, which further comprises simultaneous, separate, or sequential administration of an effective amount of a second compound, wherein the second compound is one or more chemotherapeutic agent.

Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions

The following definitions are provided to assist in understanding the scope of this disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropoisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropoisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated, unless otherwise noted.

For example,

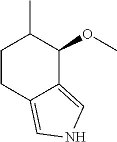

represents

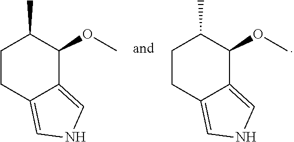

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole.

Similarly, for example, the chemical name 7-chloro-6-fluoro-1-(2-isopropyl-4-methylpyridin-3-yl)pyrido[2,3-d]pyrimidine-2,4(1H,3H)-dione represents (M)-7-chloro-6-fluoro-1-(2-isopropyl-4-methylpyridin-3-yl)pyrido[2,3-d]pyrimidine-2,4(1H,3H)-dione and (P)-7-chloro-6-fluoro-1-(2-isopropyl-4-methylpyridin-3-yl)pyrido[2,3-d]pyrimidine-2,4(1H,3H)-dione.

In certain instances, a bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropoisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. Further, this disclosure encompasses pharmaceutical compositions comprising mixtures of any of the compounds disclosed herein and one or more other active agents disclosed herein. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula.

### Isotopically-Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as 2H and 3H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically-labeled forms thereof or a pharmaceutically acceptable salt of any of the foregoing may exist in solvated or unsolvated forms.

The term “solvate” as used herein refers to a molecular complex comprising a compound, or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically-labeled forms thereof or a pharmaceutically acceptable salt of any of the foregoing.

### Miscellaneous Definitions

This section will define additional terms used to describe the scope of the compounds, compositions and uses disclosed herein.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S— nitro, cyano, carboxy, alkyl-O—C(O)—, carbamoyl, alkyl-S(O)—, sulfonyl, sulfonamido, phenyl, and heterocyclyl.

The terms “C₁₋₄alkyl,” and “C₁₋₆alkyl” as used herein refer to a straight or branched chain hydrocarbon containing from 1 to 4, and 1 to 6 carbon atoms, respectively. Representative examples of C₁₋₄alkyl or C₁₋₆ alkyl include, but are not limited to, methyl, ethyl, n-propyl, iso-propyl, n-butyl, sec-butyl, iso-butyl, tert-butyl, pentyl and hexyl.

The terms “C₁₋₄alkylene” and “C₁₋₆alkylene” refer to a straight or branched divalent alkyl group as defined herein containing 1 to 4, and 1 to 6 carbon atoms, respectively. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene and the like.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₂₋₄alkynyl” as used herein refers to a saturated hydrocarbon containing 2 to 4 carbon atoms having at least one carbon-carbon triple bond. The term includes both straight and branched moieties. Representative examples of C₃₋₆alkynyl include, but are not limited to, ethynyl, 1-propynyl, 2-propynyl, 2-butynyl and 3-butynyl.

The term “C₁₋₄alkoxy” or “C₁₋₆alkoxy” as used herein refers to —OR^{g}, wherein R^{g} represents a C₁₋₄alkyl group or C₁₋₆alkyl group, respectively, as defined herein. Representative examples of C₁₋₄alkoxy include, but are not limited to, methoxy, ethoxy, propoxy, iso-propoxy, and butoxy. Representative examples of C₁₋₆alkoxy include, but are not limited to, ethoxy, propoxy, iso-propoxy, and butoxy

The term “C₃₋₈cycloalkyl” as used herein refers to a saturated carbocyclic molecule wherein the cyclic framework has 3 to 8 carbons. Representative examples of C₃₋₈cycloalkyl include, but are not limited to, cyclopropyl and cyclobutyl.

The term “deutero” as used herein as a prefix to another term for a chemical group refers to a modification of the chemical group, wherein one or more hydrogen atoms are substituted with deuterium (“D” or “²H”). For example, the term “C₁₋₄deuteroalkyl” refers to a C₁₋₄alkyl as defined herein, wherein one or more hydrogen atoms are substituted with D. Representative examples of C₁₋₄deuteroalkyl include, but are not limited to, —CH₂D, —CHD₂, —CD₃, —CH₂CD₃, —CDHCD₃, —CD₂CD₃, —CH(CD₃)₂, —CD(CHD₂)₂, and —CH(CH₂D)(CD₃).

The term “halogen” as used herein refers to —F, —Cl, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₄haloalkyl” refers to a C₁₋₄alkyl as defined herein, wherein one or more hydrogen atoms are substituted with a halogen. Representative examples of C₁₋₄haloalkyl include, but are not limited to, —CH₂F, —CHF₂, —CF₃, —CHFCl, —CH₂CF₃, —CFHCF₃, —CF₂CF₃, —CH(CF₃)₂, —CF(CHF₂)₂, and —CH(CH₂F)(CF₃).

As used herein, the term “heteroaryl” refers to a 5-20 membered monocyclic- or bicyclic- or tricyclic-aromatic ring system, having 1 to 8 heteroatoms selected from N, O and S. In certain preferred aspects, the heteroaryl is a 5-10 membered ring system (e.g., 5-7 membered monocycle, an 8-10 membered bicycle or a 11-14 membered tricycle) or a 5-7 membered ring system. Exemplary monocyclic heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, and 5-pyrimidinyl. Exemplary bicyclic heteroaryl groups include 1-, 3-, 4-, 5-, 6-, 7-, or 8-isoquinolinyl, 2-, 3-, 4-, 5-, 6-, 7-, or 8-quinolinyl, 1-, 3-, 4-, 5-, 6-, 7-, or 8- isoquinolinyl, 1-, 2-, 4-, 5-, 6-, 7-, or 8-benzimidazolyl and 1-, 2-, 3-, 4-, 5-, 6-, 7-, or 8-indolyl.

The term “heteroaryl” also refers to a group in which a heteroaromatic ring is fused to one or more aryl, cycloaliphatic, or heterocyclyl rings.

As used herein, the term “heterocycle,” “heterocycloalkyl” or “heterocyclo” refers to a saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached to a heteroatom or a carbon atom. The heterocyclyl can include fused or bridged rings as well as spirocyclic rings. Examples of heterocycles include tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, azetidine, thiazolidine, morpholine, and the like.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following schemes are the variables as defined for Formula I, unless otherwise noted. All starting materials are either commercially available, for example, from Merck Sigma-Aldrich Inc., Fluorochem Ltd, and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein.

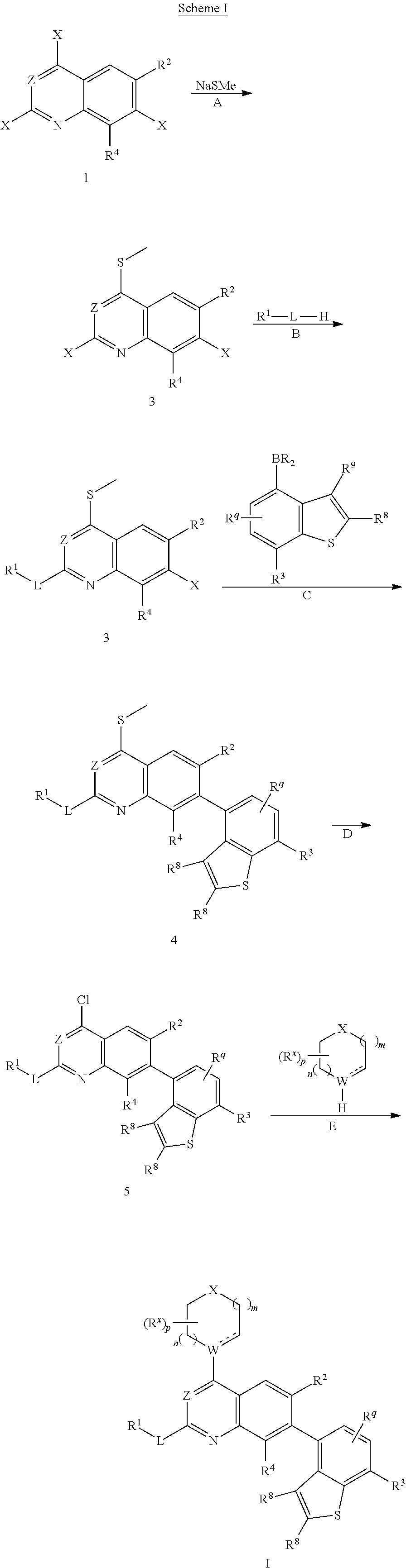

Compounds of Formula (I) can be prepared according to Scheme I. In step A, compound (1) is treated with sodium thiomethoxide in a solvent such as tetrahydrofuran to give compound (2). In step B, compound (2) is either pre-treated with a fluoride source such as potassium fluoride, or directly undergoes S_{N}Ar reaction with a nucleophile having the formula R¹-L-H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (3). In step C, compound (3) is coupled with an organometallic reagent derived from benzothiophene such as a boronic acid (ester) to provide compound (4). This coupling reaction proceeds in a solvent such as 1,4-dioxane and a catalyst such as Pd₂(dba)₃ and (R)-MOP, with or without a base such as potassium phosphate. In step D, compound (4) is treated with sulfuryl chloride in a solvent such as dichloromethane to give compound (5). In step E, compound (5) undergoes S_{N}Ar reaction with optionally substituted amine in a solvent such as acetonitrile and in the presence of a base such as N,N-diisopropylethylamine to give compound (6). In some cases, the species R⁸ will contain protecting group(s), which can be removed after step E in the synthetic sequence.

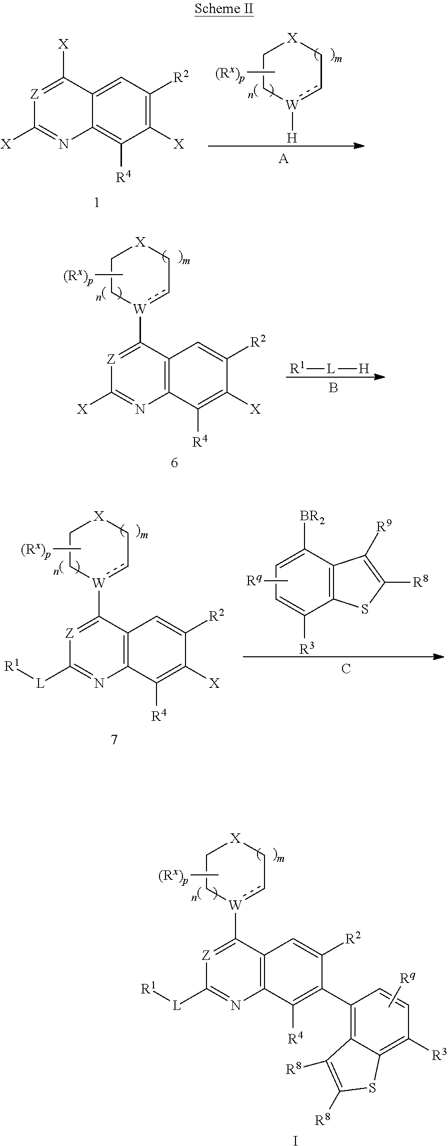

Compounds of Formula (I) can be prepared according to Scheme II. In step A, compound (1) undergoes S_{N}Ar reaction with optionally substituted amine in a solvent such as acetonitrile and in the presence of a base such as N,N-diisopropylethylamine to give compound (6). In step B, compound (6) is either pre-treated with a fluoride source such as potassium fluoride, or directly undergoes S_{N}Ar reaction with a nucleophile having the formula R¹-L-H in a solvent such as dimethylsulfoxide, or mixture of solvents such as tetrahydrofuran and N,N-dimethylforamide, in the presence of a base such as sodium hydride or cesium carbonate, with or without a nucleophilic catalyst such as 1,4-diazabicyclo[2.2.2]octane, to give compound (7). In step C, compound (7) is coupled with an organometallic reagent derived from benzothiophene such as a boronic acid (ester) to provide compound (I). This coupling reaction proceeds in a solvent such as 1,4-dioxane and a catalyst such as Pd₂(dba)₃ and (R)-MOP, with or without a base such as potassium phosphate. In some cases, the species R⁸ will contain protecting group(s), which can be removed after step C in the synthetic sequence.

### EXAMPLES

This section provides specific examples of compounds of Formula I and methods of making the same.

### LIST OF ABBREVIATIONS

TABLE 1

| | |
|---|---|
| Ac | acetyl |
| AcOH | acetic acid |
| aq or aq. | aqueous |
| Bn | benzyl |
| B₂pin₂ | bis(pinacolato)diboron |
| BOC or Boc | tert-butyloxycarbonyl |
| BrettPhos Pd G4 | (SP-4-3)-[dicyclohexyl[3,6-dimethoxy-2′,4′,6′-tris(1- |
| | methylethyl)[1,1′-biphenyl]-2-yl]phosphine- |
| | κP](methanesulfonato-κO)[2′-(methylamino-κN)[1,1′- |
| | biphenyl]-2-yl-κC]palladium |
| cataCXium A Pd G3 | mesylate[(di(1-adamanty1)-n-butylphosphine)-2-(2′- |
| | amino-1,1′-biphenyl)]palladium(II) |
| cataCXium A Pd G4 | [butylbis(tricyclo[3.3.1.1^{3,7}]dec-1- |
| | yl)phosphine](methanesulfonato-κO)[2′-(methylamino- |
| | κN)[1,1′-biphenyl]-2-yl-κC]-palladium |
| Cbz | benzyloxycarbonyl |
| CbzCl | benzyl chloroformate |
| COD or cod | 1,5-cyclooctadiene |
| DABCO | 1,4-diazabicyclo[2.2.2]octane |
| dba | dibenzylideneacetone |
| DBU | 1,8-diazabicyclo[5.4.0]undec-7-ene |
| DCM | dichloromethane |
| DEA | diethylamine |
| DMA | N,N-dimethylacetamide |
| DMF | N,N-dimethylformamide |
| DMSO | dimethyl sulfoxide |
| Dppf, DPPF or dppf | 1,1′-bis(diphenylphosphino)ferrocene |
| Dtbbpy | 4,4′-di-tert-butyl-2,2′-dipyridyl |
| eq or eq. or equiv. | equivalent |
| ESI or ES | electrospray ionization |
| Et | ethyl |
| EtOAc | ethyl acetate |
| g | gram(s) |
| h | hour(s) |
| HATU | 1-[bis(dimethylamino)methylene]-1H-1,2,3- |
| | triazolo[4,5-b]pyridinium 3-oxide hexafluorophosphate |
| HBpin | 4,4,5,5-tetramethyl-1,3,2-dioxaborolane |
| HMPA | hexamethylphosphoramide |
| HOAc | acetic acid |
| HPLC | high pressure liquid chromatography |
| iPr | iso-propyl |
| iPr₂NEt or DIPEA | N-ethyl diisopropylamine (Hünig's base) |
| KOAc | potassium acetate |
| LC MS, LCMS, LC- | liquid chromatography mass spectroscopy |
| MS or LC/MS | |
| LHMDS or LiHMDS | lithium hexamethyldisilazide |
| m/z | mass divided by charge |
| Me | methyl |
| MeCN | acetonitrile |
| MeOH | methanol |
| mg | milligrams |
| min | minutes |
| mL | milliliters |
| MS | mass spectra |
| NMR | nuclear magnetic resonance |
| Pd(dppf)Cl₂•DCM, | [1,1′- |
| Pd(dppf)Cl₂ | bis(diphenylphosphino)ferrocene]dichloropalladium(II), |
| | complex with dichloromethane |
| Pd(dtbpf)Cl₂ | [1,1′-Bis(di-tert- |
| | butylphosphino)ferrocene]dichloropalladium(II) |
| Pd(PPh₃)₄ | tetrakis(triphenylphosphine)palladium(0) |
| Ph | Phenyl |
| PhMe | toluene |
| Pin | pinacolato |
| PMB | 4-methoxybenzyl |
| rbf | round-bottom flask |
| (R)-MOP | (R)-(+)-2-(diphenylphosphino)-2′-methoxy-1,1′- |
| | binaphthyl |
| RP-HPLC | reverse phase high pressure liquid chromatography |
| RT or rt or r.t. | room temperature |
| sat. or satd. | saturated |
| SFC | supercritical fluid chromatography |
| SPhos Pd G3 | (2-dicyclohexylphosphino-2′,6′-dimethoxybiphenyl) [2- |
| | (2′-amino-1,1′-biphenyl)]palladium(II) |
| | methanesulfonate |
| SPhos Pd G4 | (SP-4-3)-[dicyclohexyl(2′,6′-dimethoxy[1,1′-biphenyl]- |
| | 2-yl)phosphine-κP](methanesulfonato-κO)[2′- |
| | (methylamino-κN)[1,1′-biphenyl]-2-yl-κC]palladium |
| TBAF | tetra-n-butylammonium fluoride |
| TBDPS | tert-butyldiphenylsilyl |
| TBDPSCl | tert-butyldiphenylsilyl chloride |
| tBu | tert-butyl |
| tBuXphos Pd G3 | [(2-di-tert-butylphosphino-2′,4′,6′-triisopropyl-1,1′- |
| | biphenyl)-2-(2′-amino-1,1′-biphenyl)] palladium(II) |
| | methanesulfonate |
| TC | thiophene-2-carboxylate |
| TEA or Et₃N | triethylamine |
| Tf | trifluoromethylsulfonyl |
| TFA | trifluoroacetic acid |
| THF | tetrahydrofuran |
| TIPS | triisopropylsilyl |
| UV | Ultraviolet |
| XantPhos Pd G3 | [(4,5-bis(diphenylphosphino)-9,9-dimethylxanthene)-2- |
| | (2′-amino-1,1′-biphenyl)]palladium(II) |
| | methanesulfonate |
| XPhos | 2-dicyclohexylphosphino-2′,4′,6′-triisopropylbiphenyl |
| XPhos Pd G3 | (2-dicyclohexylphosphino-2′,4′,6′-triisopropyl-1,1′- |
| | biphenyl)[2-(2′-amino-1,1′-biphenyl)]palladium(II) |
| | methanesulfonate |

### General Analytical and Purification Methods

Provided in this section are descriptions of the general analytical and purification methods used to prepare the specific examples provided herein.

Chromatography: Unless otherwise indicated, crude product-containing residues were purified by passing the crude material or concentrate through either a Biotage or ISCO brand silica gel column pre-packed with flash silica (SiO₂) and eluting the product off the column with a solvent gradient as indicated.

Preparative HPLC Method: Where so indicated, the compounds described herein were purified via reverse phase HPLC using Waters FractionLynx semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.

Proton NMR Spectra: Unless otherwise indicated, all ¹H NMR spectra were collected on a Bruker NMR instrument at 300, 400 or 500 MHz. All observed protons are reported as parts-per-million (ppm) downfield from tetramethylsilane (TMS) using the internal solvent peak as reference. Some 1H signals may be missing due to exchange with D from MeOD, or due to signal suppression.

Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Preparation of Intermediates

### 6-Methyl-1,4-oxazepan-6-ol hydrochloride Isomer 1 (Intermediate A1) and Isomer 2 (Intermediate A2)

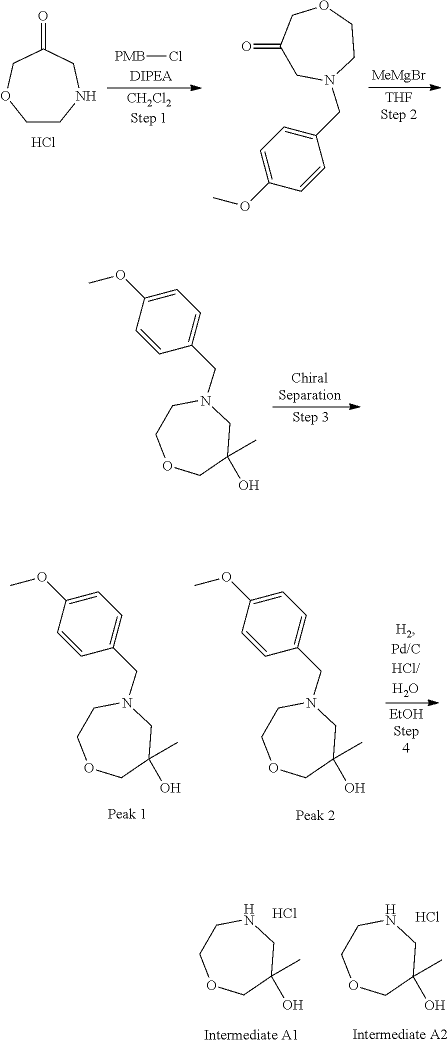

Step 1: 4-(4-Methoxybenzyl)-1,4-oxazepan-6-one. To a 20-mL vial was added 1,4-oxazepan-6-one hydrochloride (0.30 g, 1.98 mmol, AA BLOCKS LLC), 4-methoxybenzyl chloride (0.37 g, 0.32 mL, 2.38 mmol, TCI America), DIPEA (0.77 g, 1.0 mL, 5.94 mmol, Sigma-Aldrich Corporation) and DCM (10 mL). The reaction was stirred at rt overnight. The crude material was purified by column chromatography on a silica gel column, eluting with a gradient of 0-80% 3:1 EtOAc/EtOH (with 1% TEA) in heptane to provide 4-(4-methoxybenzyl)-1,4-oxazepan-6-one (0.43 g, 1.83 mmol, 92% yield) as colorless oil. m/z (ESI): 236.2 (M+H)⁺.
Step 2: 4-(4-Methoxybenzyl)-6-methyl-1,4-oxazepan-6-ol. To a 100-mL round-bottom flask was added 4-(4-methoxybenzyl)-1,4-oxazepan-6-one (0.87 g, 3.70 mmol) in THF (15 mL). The mixture was cooled to 0° C. before methylmagnesium bromide solution (3 M in Et₂O, 3.7 mL, 11.1 mmol, Sigma-Aldrich Corporation) was added. The reaction was stirred for 1 h. The reaction mixture was diluted with saturated NH₄Cl (15 mL) and extracted with EtOAc (2×15 mL). The organic extract was washed with saturated NaCl (15 mL) and dried over MgSO₄. The solution was filtered and concentrated in vacuo to give the crude material. The crude material was purified by column chromatography on silica gel, eluting with a gradient of 0-50% 3:1 EtOAc/EtOH in heptane to provide 4-(4-methoxybenzyl)-6-methyl-1,4-oxazepan-6-ol (0.65 g, 2.59 mmol, 70% yield) as yellow oil. m/z (ESI): 252.1 (M+H)⁺.
Step 3: Chiral separation. 4-(4-Methoxybenzyl)-6-methyl-1,4-oxazepan-6-ol (0.65 g, 2.59 mmol) was purified via SFC using a Chiralpak AD, 30×150 mm, 5 μm, column with a mobile phase of 20% methanol with 0.2% triethylamine using a flowrate of 200 mL/min to generate 246 mg of peak 1 with an ee of >99% and 292 mg of peak 2 with an ee of >99%.
Step 4:6-Methyl-1,4-oxazepan-6-ol hydrochloride. 4-(4-Methoxybenzyl)-6-methyl-1,4-oxazepan-6-ol (0.24 g, 0.96 mmol, Peak 1) was dissolved in ethanol (5.8 mL). Palladium on activated carbon (0.25 g, 0.23 mmol, Sigma-Aldrich Corporation) and aqueous HCl solution (2 N, 0.7 mL, 1.33 mmol, Sigma-Aldrich Corporation) were added and the mixture stirred at rt under an atmosphere of H₂ for 5 h. The catalyst was removed, and the solution was concentrated to provide 6-methyl-1,4-oxazepan-6-ol hydrochloride (quant, yield, isomer 1, Intermediate A1). Isomer 2, Intermediate A2 was obtained by the same method.

### 6-(Difluoromethyl)-1,4-oxazepan-6-ol hydrochloride Isomer 1 (Intermediate B1) and Isomer 2 (Intermediate B2)

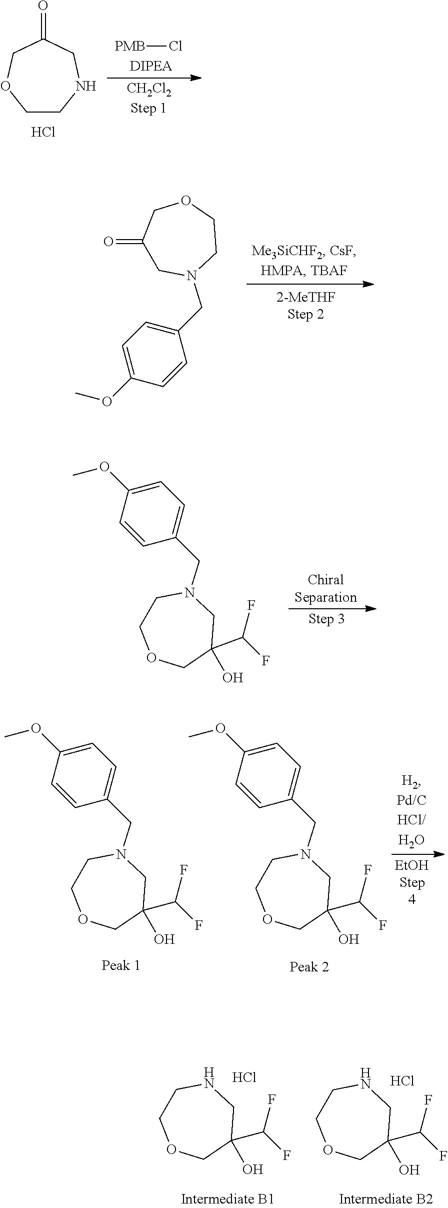

Synthesized in an analogous manner to Intermediates A1 and A2, with modification to Step 2: To a solution of 4-(4-methoxybenzyl)-1,4-oxazepan-6-one (0.97 g, 4.14 mmol) in 2-MeTHF (20 mL) at rt was added HMPA (3.70 g, 3.70 mL, 20.68 mmol, Sigma-Aldrich Corporation) and cesium fluoride (0.31 g, 2.07 mmol, Sigma-Aldrich Corporation), followed by (difluoromethyl) trimethylsilane (1.50 g, 1.7 mL, 12.41 mmol, Oakwood Products, Inc.). The resulting mixture was stirred at rt overnight. To the reaction mixture was added tetrabutylammonium fluoride solution (1.0 M in THF, 8.3 mL, 8.27 mmol, Sigma-Aldrich Corporation) and the resulting mixture was stirred at rt for 1 h. The reaction mixture was diluted with water and extracted with DCM. The organic layer was concentrated and purified by column chromatography, eluting with a gradient of 0-60% 3:1 EtOAc/EtOH with 0.2% Et₃N in heptane to afford 6-(difluoromethyl)-4-(4-methoxybenzyl)-1,4-oxazepan-6-ol (0.45 g, 1.57 mmol, 38% yield) as light brownish oil which was used directly without further purification. m/z (ESI): 288.0 (M+H)⁺.

### Conditions for Chiral SFC Separation:

| Separation | SFC Conditions | Peak to Int.# |
|---|---|---|
| 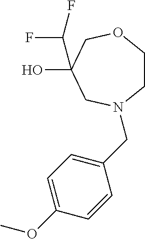 [SMILES: COc1ccc(CN2CCOCC(O)(C(F)F)C2)cc1] | Column: ChiralPak AD (21 × 250 mm, 5 μm). Mobile phase: 35% MeOH with 0.2% Et₃N. Flowrate: 80 mL/min. Yield: 545 mg sample was submitted to generate 73 mg of peak 1 with an ee of 99% and 87 mg of peak 2 with an ee of 96%. | Peak 1: Intermediate B1 Peak 2: Intermediate B2 |

### 6-Ethyl-1,4-oxazepan-6-ol hydrochloride Isomer 1 (Intermediate C1) and Isomer 2 (Intermediate C2)

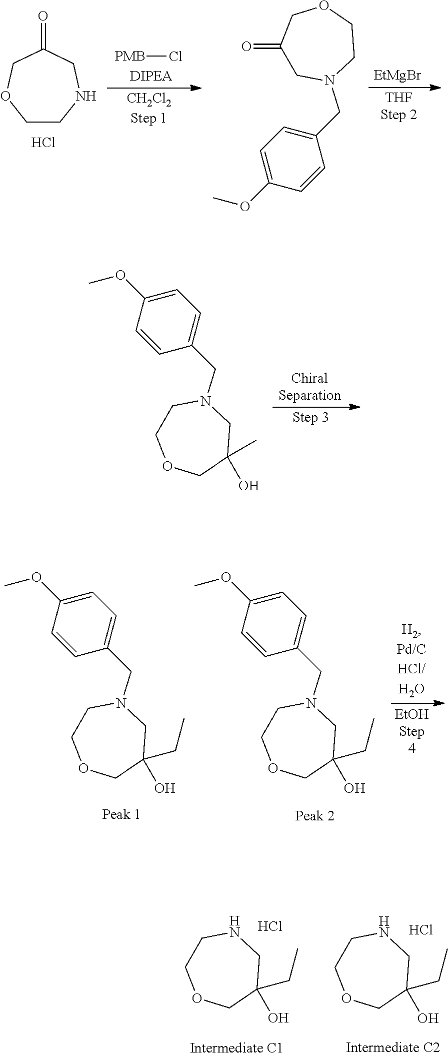

Synthesized in an analogous manner to Intermediates A1 and A2.

### Conditions for Chiral SFC Separation:

| Separation | SFC Conditions | Peak to Int.# |
|---|---|---|
| 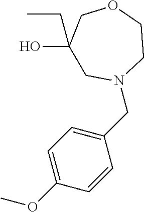 [SMILES: CCC1(O)COCCN(Cc2ccc(OC)cc2)C1] | Column: ChiralPak AZ, 2 × 25 cm, 5 μm. Mobile phase: 15% MeOH. Flowrate: 80 mL/min. Yield: 400 mg sample was submitted to generate 163 mg of peak 1 with an ee of 99% and 163 mg of peak 2 with an ee of 99%. | Peak 1: Intermediate C1 Peak 2: Intermediate C2 |

### 2,3,6,7-Tetrahydro-1H-azepin-3-ol hydrochloride (Intermediate D)

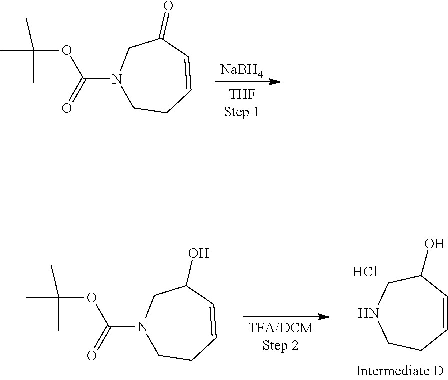

Step 1: tert-Butyl 3-hydroxy-2,3,6,7-tetrahydro-1H-azepine-1-carboxylate. To a 25-mL round-bottom flask was added tert-butyl 3-oxo-2,3,6,7-tetrahydro-1H-azepine-1-carboxylate (75 mg, 0.36 mmol, AstaTech, Inc) and sodium borohydride (40 mg, 1.07 mmol, Sigma-Aldrich Corporation) in THF (1.8 mL). A small amount of MeOH was added. The reaction was stirred at rt for 1 h. The reaction mixture was diluted with sat'd NH₄Cl (10 mL) and extracted with EtOAc (2×10 mL). The organic extract was washed with sat'd NaCl (10 mL) and dried over MgSO₄. The solution was filtered and concentrated in vacuo to give the crude material, which was used directly in the subsequent step. m/z (ESI): 236.2 (M+Na)⁺.
Step 2: 2,3,6,7-Tetrahydro-1H-azepin-3-ol hydrochloride. The above mentioned material was dissolved in DCM (2 mL). TFA (2 mL) was added to the mixture at 0° C. The reaction was stirred for 1 h. The mixture was fully concentrated under reduced pressure. 1 N HCl (1.0 mL) was added, and the material was lyophilized to give 2,3,6,7-tetrahydro-1H-azepin-3-ol hydrochloride.

### 3-Methylazepan-3-ol hydrochloride Isomer 1 (Intermediate E1) and Isomer 2 (Intermediate E2)

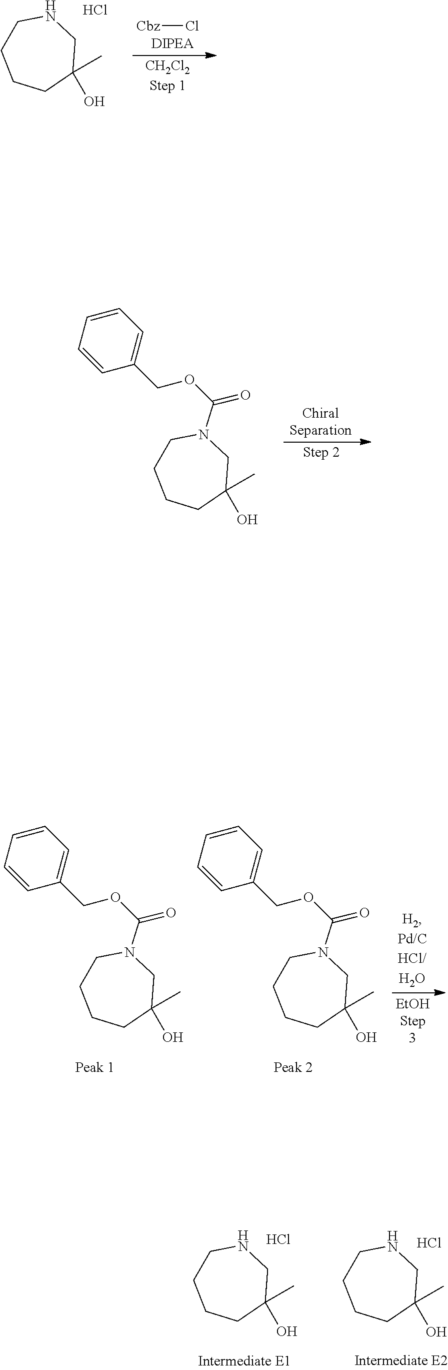

Step 1: Benzyl 3-hydroxy-3-methylazepane-1-carboxylate. To a 100-mL round-bottomed flask was added 3-methylazepan-3-ol hydrochloride (0.25 g, 1.51 mmol, Asta-tech), benzyl chloroformate (0.28 g, 0.24 mL, 1.66 mmol, Oakwood Products, Inc.), DIPEA (0.59 g, 0.8 mL, 4.53 mmol, Sigma-Aldrich Corporation) and THF (5.0 mL). The reaction was stirred at rt overnight. The solution was concentrated in vacuo. The crude material was purified by column chromatography on silica gel column, eluting with a gradient of 0-100% EtOAc in hexane to provide benzyl 3-hydroxy-3-methylazepane-1-carboxylate (0.33 g, 1.25 mmol, 83% yield) as colorless oil. m/z (ESI): 264.2 (M+H)⁺.
Step 2: Chiral separation. Benzyl 3-hydroxy-3-methylazepane-1-carboxylate (0.24 g) was purified via SFC using a Chiralpak IG, 21×250 mm, 5 μm, column with a mobile phase of 20% methanol with 0.2% triethylamine using a flowrate of 80 mL/min to generate 89 mg of peak I with an ee of >99% and 91 mg of peak 2 with an ee of >99%.
Step 3: 3-Methylazepan-3-ol hydrochloride. Benzyl 3-hydroxy-3-methylazepane-1-carboxylate (89 mg, 0.34 mmol, Peak 1) was dissolved in ethanol (1.7 mL). Palladium on activated carbon (74 mg, 0.07 mmol, Sigma-Aldrich Corporation) and aqueous HCl solution (1 N, 0.2 mL, 0.42 mmol, Sigma-Aldrich Corporation) were added and the mixture stirred at rt under an atmosphere of H₂ for 10 h. The catalyst was removed, and the solution was concentrated to provide 3-methylazepan-3-ol hydrochloride (isomer 1, Intermediate E1, 60 mg, 0.36 mmol). Isomer 2, Intermediate E2 was obtained by the same method. m/z (ESI): 130.2 (M+H)⁺.

### tert-Butyl (4-(6-chloro-8-fluoro-2-(((2R,7aS)-2-fluorotetrahydro-1H-pyrrolizin-7a(5H)-yl)methoxy)-4-(methylthio)quinazolin-7-yl)-3-cyano-7-fluorobenzo[b]thiophen-2-yl)carbamate (Intermediate F, F1, F2)

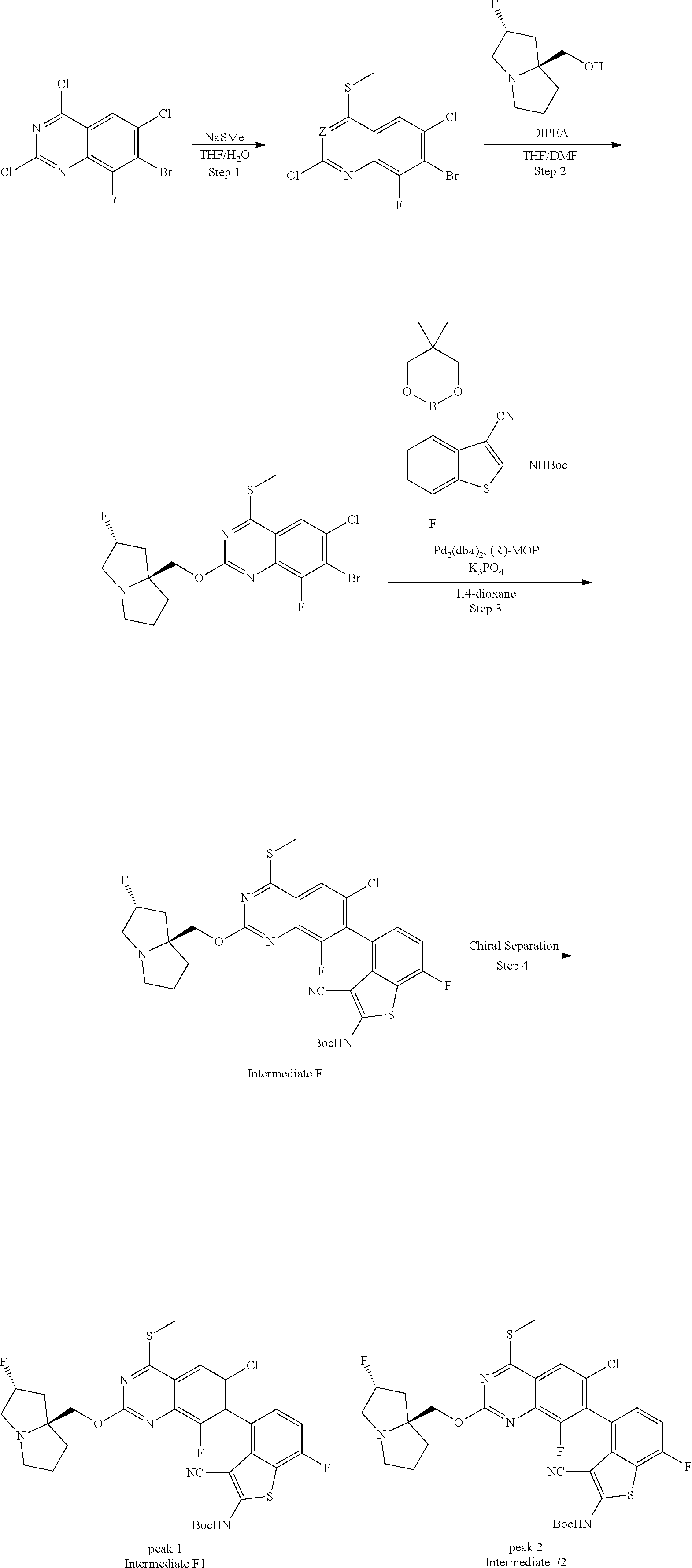

Step 1: 7-Bromo-2,6-dichloro-8-fluoro-4-(methylthio)quinazoline. To a stirring solution of 7-bromo-2,4,6-trichloro-8-fluoroquinazoline (2.30 g, 6.96 mmol, Advanced ChemBlocks Inc.) in THF (25 mL) at 0° C. was added sodium methanethiolate (0.54 g, 7.66 mmol, Sigma-Aldrich Corporation) in water (6.0 mL). The reaction was then stirred at rt for 1 h. The precipitate was collected by filtration and washed with heptane to afford 7-bromo-2,6-dichloro-8-fluoro-4-(methylthio)quinazoline (1.60 g, 4.68 mmol, 67% yield) as off-white solid. m/z (ESI): 342.8 (M+H)⁺.
Step 2: 7-Bromo-6-chloro-8-fluoro-2-(((2R,7a5)-2-fluorotetrahydro-1H-pyrrolizin-7a(5H)-yl)methoxy)-4-(methylthio)quinazoline. A mixture of 7-bromo-2,6-dichloro-8-fluoro-4-(methylthio)quinazoline (1.60 g, 4.68 mmol), ((2R,7aS)-2-fluorotetrahydro-1H-pyrrolizin-7a(5H)-yl)methanol (1.12 g, 7.02 mmol, LabNetwork), and DIPEA (2.42 g, 3.27 mL, 18.71 mmol, Sigma-Aldrich Corporation) was stirred in acetonitrile (20 mL) at 80° C. overnight. The precipitate was collected by filtration and washed with heptane to obtain 7-bromo-6-chloro-8-fluoro-2-(((2R,7aS)-2-fluorotetrahydro-1H-pyrrolizin-7a(5H)-yl)methoxy)-4-(methylthio)quinazoline (1.18 g, 2.55 mmol, 54% yield) as orange solid. m/z (ESI): 464.8 (M+H)⁺.
Step 3: tert-Butyl (4-(6-chloro-8-fluoro-2-(((2R,7aS)-2-fluorotetrahydro-1H-pyrrolizin-7a(5H)-yl)methoxy)-4-(methylthio)quinazolin-7-yl)-3-cyano-7-fluorobenzo[b]thiophen-2-yl)carbamate. Potassium phosphate (0.91 g, 4.30 mmol, Combi-Blocks Inc.), (R)-(+)-2-(diphenylphosphino)-2′-methoxy-1,1′-binaphthyl (81 mg, 0.17 mmol, Strem Chemicals, Inc.), bis[tris(dibenzylideneacetone)palladium(0)] (79 mg, 0.09 mmol, Sigma-Aldrich Corporation), 7-bromo-6-chloro-8-fluoro-2-(((2R,7aS)-2-fluorotetrahydro-1H-pyrrolizin-7a(5H)-yl)methoxy)-4-(methylthio)quinazoline (0.40 g, 0.86 mmol), and tert-butyl (3-cyano-4-(5,5-dimethyl-1,3,2-dioxaborinan-2-yl)-7-fluorobenzo[b]thiophen-2-yl)carbamate (0.70 g, 1.72 mmol, PharmaBlock) were mixed in 1,4-dioxane (6.8 mL). The reaction mixture was stirred at 80° C. for 1 h. The crude mixture was purified by column chromatography on silica gel, eluting with a gradient of 0-50% 3:1 EtOAc/EtOH in heptane with 2% triethylamine as additive to yield tert-butyl (4-(6-chloro-8-fluoro-2-(((2R,7aS)-2-fluorotetrahydro-1H-pyrrolizin-7a(5H)-yl)methoxy)-4-(methylthio)quinazolin-7-yl)-3-cyano-7-fluorobenzo[b]thiophen-2-yl)carbamate (Intermediate F, 0.24 g, 0.35 mmol, 41% yield). m/z (ESI): 675.7 (M+H)⁺.

### Step 4: Chiral Separation. Conditions for Chiral SFC Separation

| Separation | SFC Conditions | Peak to AMG# |
|---|---|---|
| 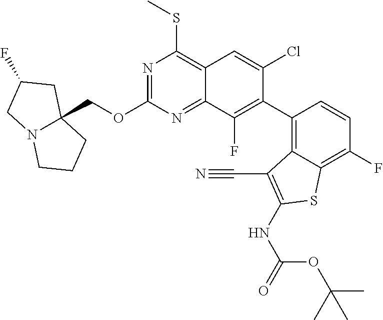 [SMILES: CSc1nc(OC[C@@]23CCCN2C[C@H](F)C3)nc2c(F)c(-c3ccc(F)c4sc(NC(=O)OC(C)(C)C)c(C#N)c34)c(Cl)cc12] | Column: ChromegaChiral CC4 (21 × 250 mm, 5 μm) Mobile phase: 35% MeOH with diethylamine Flowrate: 100 mL/min. Yield: 827 mg sample was submitted to generate 264 mg of peak 1 with an ee of 99% and 302 mg of peak 2 with an ee of 96%. | Peak 1: Intermediate F1 Peak 2: Intermediate F2 |

### Experimental Procedures

### 2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (Example 1)

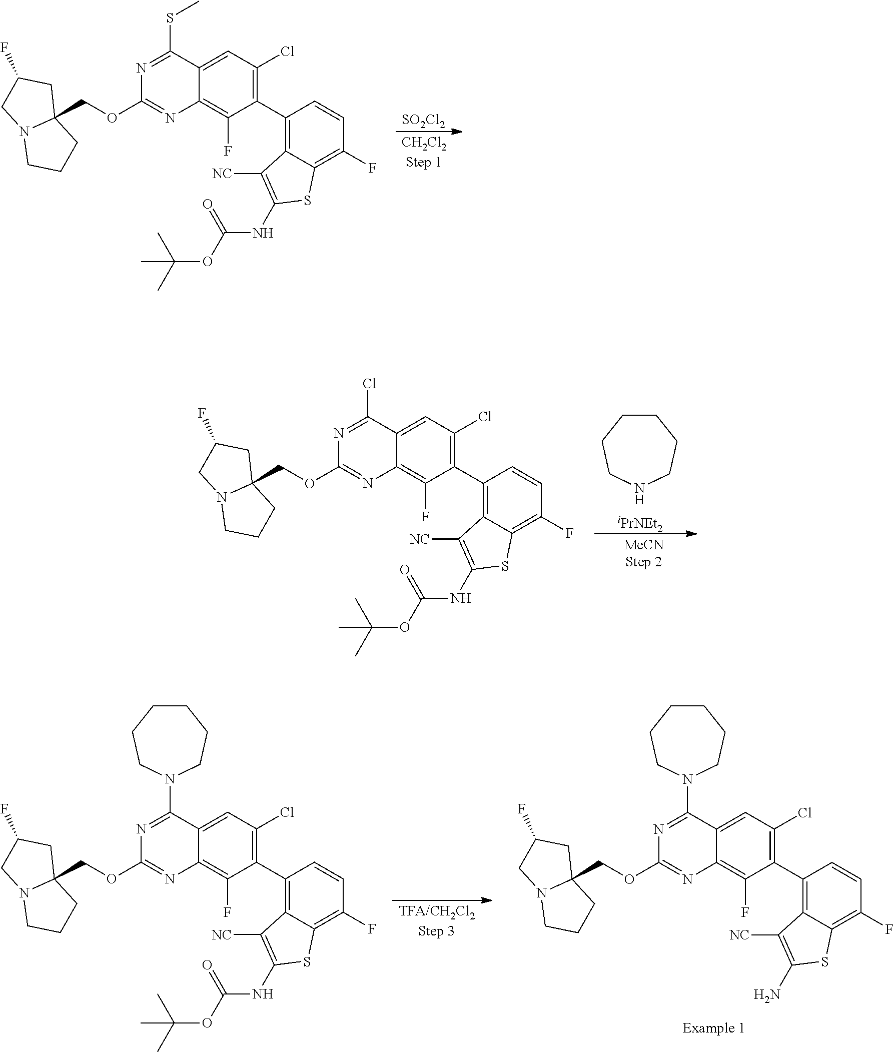

Step 1: tert-Butyl (3-cyano-4-(4,6-dichloro-8-fluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate. To a solution of tert-butyl (4-(6-chloro-8-fluoro-2-(((2R,7aS)-2-fluorotetrahydro-1H-pyrrolizin-7a(5H)-yl)methoxy)-4-(methylthio)quinazolin-7-yl)-3-cyano-7-fluorobenzo[b]thiophen-2-yl)carbamate (57 mg, 0.08 mmol, Intermediate F) in DCM (2.0 mL) at 0° C. was added sulfuryl chloride (1.0 M in DCM, 0.25 mL, 0.25 mmol, Sigma-Aldrich Corporation) slowly. The reaction mixture was stirred at 0° C. for 1 h. The reaction mixture was concentrated under reduced pressure without heating. The crude product was used directly in the next step. m/z (ESI): 664.0 (M+H)⁺.
Step 2: tert-Butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate. To a solution of tert-butyl (3-cyano-4-(4,6-dichloro-8-fluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate (19 mg, 0.03 mmol) in acetonitrile (1.0 mL) was added DIPEA (11 mg, 0.08 mmol, Sigma-Aldrich Corporation) and hexamethyleneimine (5.6 mg, 5.6 μL, 0.06 mmol, Combi-Blocks Inc.). The resulting mixture was stirred at rt for 1 h. The reaction mixture was purified by reverse phase HPLC to afford tert-butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (14 mg, 0.02 mmol, 69%) as light yellow solid. m/z (ESI): 727.2 (M+H)⁺.
Step 3: 2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile. tert-Butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (14 mg, 0.02 mmol) was dissolved in DCM (0.5 mL) and trifluoroacetic acid (0.5 mL). The reaction mixture was stirred at rt for 1 h. The reaction mixture was concentrated under reduced pressure. The crude product was purified by reverse phase HPLC to afford 2-amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile as bis(2,2,2-trifluoroacetate) and as light yellow solid (8.1 mg, 9.47 μmol, 34%). m/z (ESI): 627.0 (M+H)⁺. ¹H NMR (400 MHz, METHANOL-d₄) δ ppm 8.13 (d, J=1.9 Hz, 1H), 7.22-7.27 (m, 1H), 7.07 (dd, J=9.3, 8.5 Hz, 1H), 5.42-5.68 (m, 1H), 4.54-4.75 (m, 2H), 4.04-4.12 (m, 4H), 3.83-4.01 (m, 3H), 3.43-3.52 (m, 1H), 2.54-2.72 (m, 2H), 2.31-2.48 (m, 3H), 2.10-2.22 (m, 1H), 2.06 (br s, 4H), 1.69-1.78 (m, 4H).

TABLE 2
Additional Examples 2 to 54, 65 to 67. Prepared in an Analogous Manner to Example 1.

| Ex. | | | Salt | | Method |
|---|---|---|---|---|---|
| # | Structure | Name | Form | Reagent | Change |
| 2 | 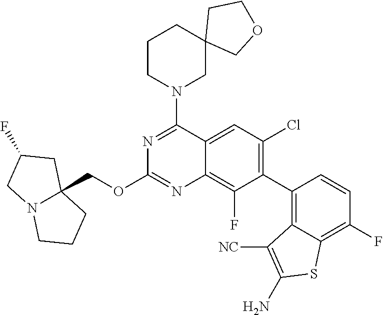 [SMILES: N#Cc1c(N)sc2c(F)ccc(-c3c(Cl)cc4c(N5CCCC6(CCOC6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrolizin- 7a(5H)- yl)methoxy)-4- (2-oxa-7- azaspiro[4.5]decan- 7-yl)quinazolin- 7-yl)-7- fluorobenzo[b] thiophene-3- carbonitrile | Bis(2, 2,2- trifluo- roacetate) | Step 2: 2-oxa- 7-azaspiro[4.5] decane hydrochloride (CAS#: 374795-37-6, Enamine) | Crude product from Step 2 was carried to Step 3 after concentration without purification. |
| 3 | 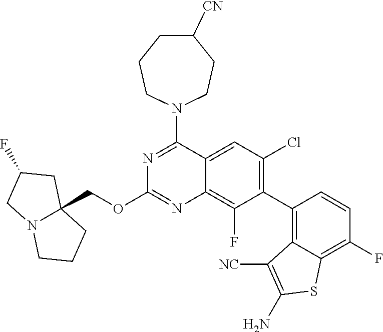 [SMILES: N#Cc1c(N)sc2c(F)ccc(-c3c(Cl)cc4c(N5CCCC(C#N)CC5)nc(OC[C@@]56CCCN5C[C@H](F)C6)nc4c3F)c12] | 1-(7-(2-Amino-3- cyano-7- fluorobenzo[b] thiophen-4-yl)-6- chloro-8-fluoro- 2-(((2R,7a.S)-2- fluorotetrahydro- 1H-pyrrolizin- 7a(5H)- yl)methoxy)quina- zolin-4- yl)azepane-4- carbonitrile | Bis(2, 2,2- trifluoro- acetate) | Step 2: azepane-4- carbonitrile hydrochloride (CAS#: 1259062-50-4, AstaTech, Inc) | Crude product from Step 2 was carried to Step 3 after concentration without purification. |
| 4 | 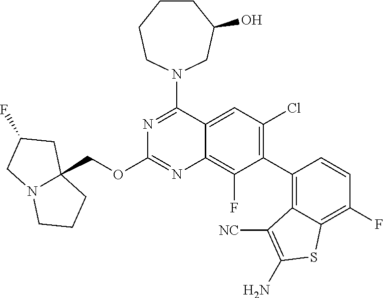 [SMILES: N#Cc1c(N)sc2c(F)ccc(-c3c(Cl)cc4c(N5CCCC[C@@H](O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- ((R)-3- hydroxyazepan- 1-yl)quinazolin- 7-yl)-7- fluorobenzo[b] thiophene-3- carbonitrile | Bis(2, 2,2- trifluoro- acetate) | Step 2: (R)- azepan-3-ol (CAS#: 1956435-25-8, J&W Pharmlab) | Crude product from Step 2 was carried to Step 3 after concentration without purification. |
| 5 | 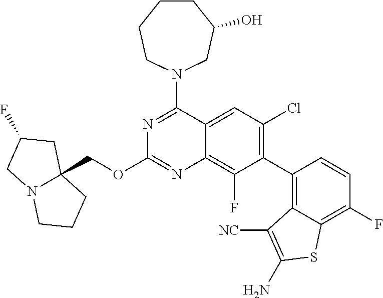 [SMILES: N#Cc1c(N)sc2c(F)ccc(-c3c(Cl)cc4c(N5CCCC[C@H](O)C5)nc(OC[C@@]56CCCN5C[C@H](F)C6)nc4c3F)c12] | 2-Amino-4-(6- chloro-8-fluoro- 2-(((2R,7a.S)-2- fluorotetrahydro- 1H-pyrrolizin- 7a(5H)- yl)methoxy)-4- ((S)-3- hydroxyazepan- 1-yl)quinazolin- 7-yl)-7- fluorobenzo[b] thiophene-3- carbonitrile | Bis(2, 2.2- trifluo roacet ate) | Step 2: (S)- azepan-3-ol (CAS#: 1923069-56-0, J&W Pharmlab) | Crude product from Step 2 was carried to Step 3 after concentrati on without purification |
| 6 | 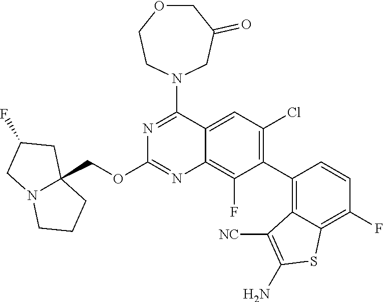 [SMILES: N#Cc1c(N)sc2c(F)ccc(-c3c(Cl)cc4c(N5CCOCC(=O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (6-oxo-1,4- oxazepan-4- yl)quinazolin-7- yl)-7- fluorobenzo[b] thiophene-3- carbonitrile | Bis(2, 2,2- trifluoro- acetate) | Step 2: 1,4- oxazepan-6- one hydrochloride (CAS#: 2306265-53-0, AA BLOCKS LLC) | Crude product from Step 2 was carried to Step 3 after concentration without purification. |
| 7 | 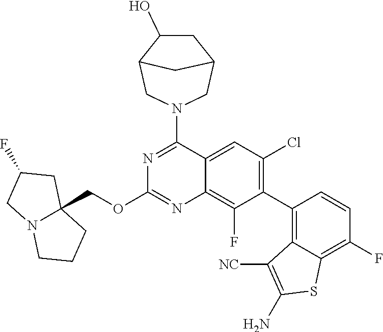 [SMILES: N#Cc1c(N)sc2c(F)ccc(-c3c(Cl)cc4c(N5CC6CC(O)C(C6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (rac-(1R,5R,6R)- 6-hydroxy-3- azabicyclo[3.2.1] octan-3- yl)quinazolin-7- yl)-7- fluorobenzo[6] thiophene-3- carbonitrile | Bis(2, 2,2- trifluoro- acetate) | Step 2: rac- (1R,5R,6R)-3- azabicyclo[3.2.1] octan-6-ol hydrochloride (CAS#: 2679949-46-1, Enamine) | Crude product from Step 2 was carried to Step 3 after concentration without purification. |
| 8 | 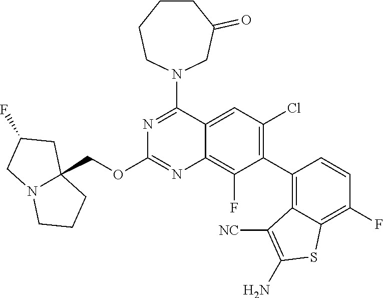 [SMILES: N#Cc1c(N)sc2c(F)ccc(-c3c(Cl)cc4c(N5CCCCC(=O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (3-oxoazepan-1- yl)quinazolin-7- yl)-7- fluorobenzo[b] thiophene-3- carbonitrile | Bis(2, 2,2- trifluoro- acetate) | Step 2: azepan-3-one hydrochloride (CAS#: 65326-54-7, Combi-Blocks Inc.) | Crude product from Step 2 was carried to Step 3 after concentration without purification |
| 9 | 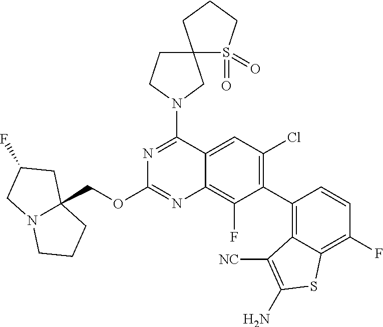 [SMILES: N#Cc1c(N)sc2c(F)ccc(-c3c(Cl)cc4c(N5CCC6(CCCS6(=O)=O)C5)nc(OC[C@@]56CCCN5C[C@H](F)C6)nc4c3F)c12] | 2-Amino-4-(6- chloro-4-(1,1- dioxido-1-thia-7- azaspiro[4.4]nonan- 7-yl)-8-fluoro- 2-(((2R,7aS)-2- fluorotetrahydro- 1H-pyrrolizin- 7a(5H)- yl)methoxy) quinazolin-7-yl)-7- fluorobenzo[b] thiophene-3- carbonitrile | Bis(2, 2,2- trifluoro- acetate) | Step 2: λ⁶- thia-7- azaspiro[4.4] nonane-1,1- dione hydrochloride (CAS#: 2089257-89-4, Enamine) | Crude product from Step 2 was carried to Step 3 after concentration without purification. |
| 10 | 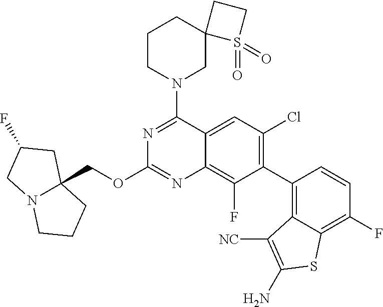 [SMILES: N#Cc1c(N)sc2c(F)ccc(-c3c(Cl)cc4c(N5CCCC6(CCS6(=O)=O)C5)nc(OC[C@@]56CCCN5C[C@H](F)C6)nc4c3F)c12] | 2-Amino-4-(6- chloro-4-(1,1- dioxido-1-thia-6- azaspiro[3.5]nonan- 6-yl)-8-fluoro- 2-(((2R,7aS)-2- fluorotetrahydro- 1H-pyrrolizin- 7a(5H)- yl)methoxy) quinazolin-7-yl)-7- fluorobenzo[b] thiophone-3- carbonitrile | Bis(2, 2,2- trifluoro- acetate) | Step 2: 1λ⁶- thia-6- azaspiro[3.5] nonane-1,1- dione hydrochloride (CAS#: 1936207-13-4, ChemSpace) | Crude product from Step 2 was carried to Step 3 after concentration without purification. |
| 11 | 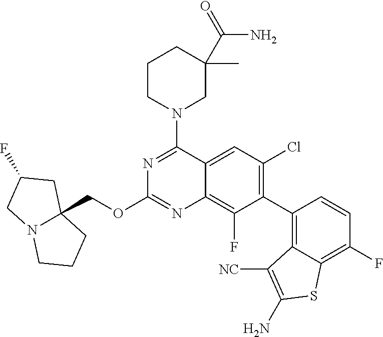 [SMILES: CC1(C(N)=O)CCCN(c2nc(OC[C@@]34CCCN3C[C@H](F)C4)nc3c(F)c(-c4ccc(F)c5sc(N)c(C#N)c45)c(Cl)cc23)C1] | 1-(7-(2-Amino-3- cyano-7- fluorobenzo[b] thiophen-4-yl)-6- chloro-8-fluoro- 2-(((2R,7aS)-2- fluorotetrahydro- 1H-pyrrolizin1 7a(5H)- yl)methoxy) quinazolin-4-yl)-3- methylpiperidine- 3-carboxamide | Bis(2, 2,2- trifluoro- acetate) | Step 2: 3- methyl-3- piperidine- carboxamide hydrochloride (CAS#: 1315499-98-9, AA BLOCKS LLC) | Crude product from Step 2 was carried to Step 3 after concentration without purification. |
| 12 | 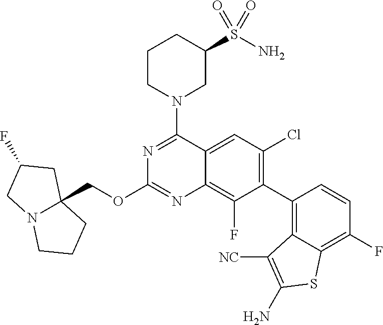 [SMILES: N#Cc1c(N)sc2c(F)ccc(-c3c(Cl)cc4c(N5CCC[C@@H](S(N)(=O)=O)C5)nc(OC[C@@]56CCCN5C[C@H](F)C6)nc4c3F)c12] | (3R)-1-(7-(2- Amino-3-cyano- 7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)piperidine-3- sulfonamide | Bis(2, 2,2- trifluoro- acetate) | Step 2: (3R)- piperidine-3- sulfonamide hydrochloride (CAS#: 2416219-36-6, Enamine) | Crude product from Step 2 was carried to Step 3 after concentration without purification. |
| 13 | 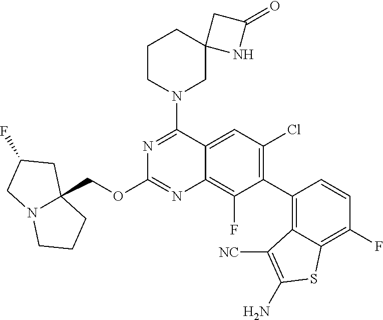 [SMILES: N#Cc1c(N)sc2c(F)ccc(-c3c(Cl)cc4c(N5CCCC6(CC(=O)N6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin1 7a(5H)- yl)methoxy)-4- (2-oxo-1,6- diazaspiro[3.5] nonan-6- yl)quinazolin-7- yl)-7- fluorobenzo[b] thiophene-3- carbonitrile | Bis(2, 2,2- trifluoro- acetate) | Step 2: 1,6- diazaspiro[3.5] nonan-2-one (CAS#: 1567085-5-7, Pharmablock, Inc.) | Crude product from Step 2 was carried to Step 3 after concentration without purification. |
| 14 | 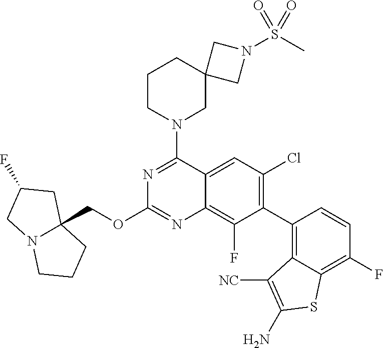 [SMILES: CS(=O)(=O)N1CC2(CCCN(c3nc(OC[C@@]45CCCN4C[C@H](F)C5)nc4c(F)c(-c5ccc(F)c6sc(N)c(C#N)c56)c(Cl)cc34)C2)C1] | chloro-8-fluoro- 2-(((2R,7aS)-2- fluorotetrahydro- 1H-pyrrolizin- 7a(5H)- yl)methoxy)-4- (2- (methylsulfonyl)- 2,6- diazaspiro[3.5] nonan-6- yl)quinazolin-7- yl)-7- fluorobenzo[b] thiophene-3- carbonitrile | Bis(2, 2,2- trifluoro- acetate) | methanesulfonyl- 2,6- diazaspiro[3.5] nonane trifluoroacetic acid (CAS#: 1360427-18-4, Enamine) | Crude product from Step 2 was carried to Step 3 after concentration without purification. |
| 15 | 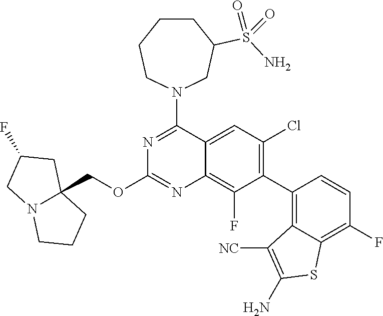 [SMILES: N#Cc1c(N)sc2c(F)ccc(-c3c(Cl)cc4c(N5CCCCC(S(N)(=O)=O)C5)nc(OC[C@@]56CCCN5C[C@H](F)C6)nc4c3F)c12] | 1-(7-(2-Amino-3- cyano-7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)azepane-3- sulfonamide | Bis(2, 2,2- trifluoro- acetate) | Step 2: azeane-3- sulfonamide hydrochloride (CAS#: 1934897-80-9, ChemSpace) | Crude product from Step 2 was carried to Step 3 after concentration without purification. |
| 16 | 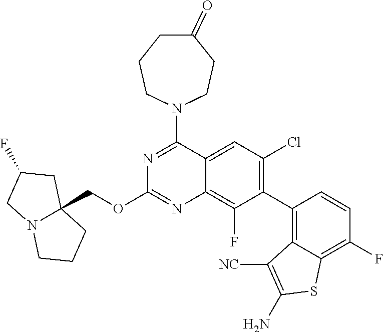 [SMILES: N#Cc1c(N)sc2c(F)ccc(-c3c(Cl)cc4c(N5CCCC(=O)CC5)nc(OC[C@@]56CCCN5C[C@H](F)C6)nc4c3F)c12] | 2-Amino-4-(6- chloro-8-fluoro- 2-(((2R,7a.S)-2- fluorotetrahydro- 1H-pyrrolizin- 7a(5H)- yl)methoxy)-4- (4-oxoazepan-1- yl)quinazolin-7- yl)-7- fluorobenzo[b] thiophene-3- carbonitrile | Bis(2, 2,2- trifluoro- acetate) | Step 2: 4- perhydro- azepinone hydrochloride (CAS#: 50492-22-3, Aagile Labs Division of Tyger Scientific) | Crude product from Step 2 was carried to Step 3 after concentration without purification. |
| 17 | 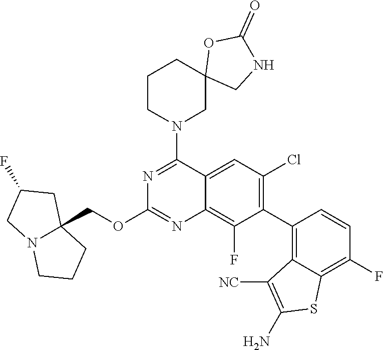 [SMILES: N#Cc1c(N)sc2c(F)ccc(-c3c(Cl)cc4c(N5CCCC6(CNC(=O)O6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (2-oxo-1-oxa-3,7- diazaspiro[4.5] decan-7- yl)quinazolin-7- yl)-7- fluorobenzo[b] thiophene-3- carbonitrile | 2,2,2- trifluoro- aceate) | Step 2: 1-oxa- 3,7- diazaspiro[4.5] decan-2-one (CAS#: 1308384-36-2, ChemSpace) | |
| 18 | 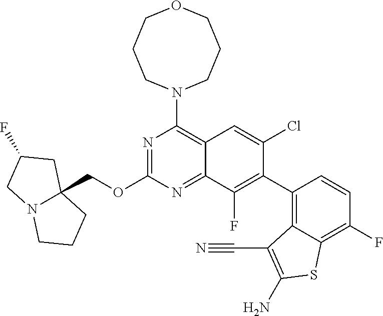 [SMILES: N#Cc1c(N)sc2c(F)ccc(-c3c(Cl)cc4c(N5CCCOCCC5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (1,5-oxazocan-5- yl)quinazolin-7- yl)-7- fluorobenzo[b] thiophene-3- carbonitrile Isomer 1 | Bis (2,2,2- trifluoro- aceate) | Step 2: 1,5- oxazocane (CAS#: 107261-17-6, Princeton BioMolecular Research, Inc.) and tert- butyl (4-(6- chloro-8- fluoro-2- (((2R,7aS)-2- fluorotetrahydro- 1H- pyrrolizin- 7a(5H)- yl)methoxy)- 4- (methylthio) quinazolin-7- | |
| | | | | yl)-3-cyano-7- | |
| | | | | fluorobenzo[b] | |
| | | | | thiophen-2- | |
| | | | | yl)carbamate | |
| | | | | (Intermediate | |
| | | | | F1) | |
| 19 | 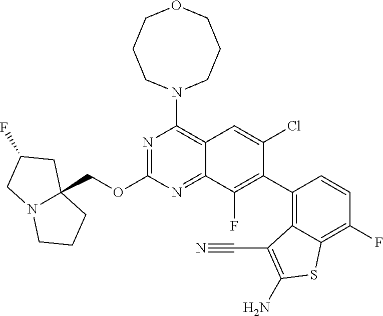 [SMILES: N#Cc1c(N)sc2c(F)ccc(-c3c(Cl)cc4c(N5CCCOCCC5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (1,5-oxazocan-5- yl)quinazolin-7- yl)-7- fluorobenzo[b] thiophene-3- carbonitrile Isomer 2 | Bis (2,2.2- trifluoro- aceate) | Step 2: 1,5- oxazocane (CAS#: 107261-17-6, Princeton BioMolecular Research, Inc.) and tert- butyl (4-(6- chloro-8- fluoro-2- (((2R,7aS)-2- fluorotetrahydro- 1H- pyrrolizin- 7a(5H)- yl)methoxy)- 4- (methylthio) quinazolin-7- | |
| | | | | yl)-3-cyano-7- | |
| | | | | fluorobenzo[b] | |
| | | | | thiophen-2- | |
| | | | | yl)carbamate | |
| | | | | (Intermediate | |
| | | | | F2) | |
| 20 | 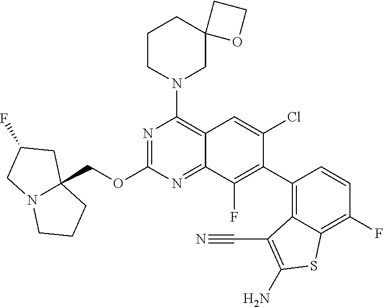 [SMILES: N#Cc1c(N)sc2c(F)ccc(-c3c(Cl)cc4c(N5CCCC6(CCO6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (1-oxa-6- azaspiro[3.5]nonan- 6-yl)quinazolin-7- yl)-7- fluorobenzo[b] thiophene-3- carbonitrile | Bis (2,2,2- trifluoro- aceate) | Step 2: 1-oxa- 6- azaspiro[3.5] nonane hydrochloride (CAS#: 1956354-49-6, Enamine) | |
| 21 | 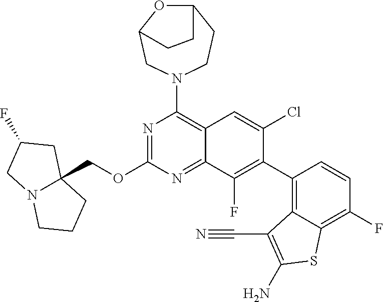 [SMILES: N#Cc1c(N)sc2c(F)ccc(-c3c(Cl)cc4c(N5CCC6CCC(C5)O6)nc(OC[C@@]56CCCN5C[C@H](F)C6)nc4c3F)c12] | tert-Butyl (4-(4- (9-oxa-3- azabicyclo[4.2.1] nonan-3-yl)-6- chloro-8-fluoro- 2-(((2R,7aS)-2- fluorotetrahydro- 1H-pyrrolizin- 7a(5H)- yl)methoxy) quinazolin-7-yl)-3- cyano-7- fluorobenzo[b] thiophen-2- yl)carbamate | Free base | Step 2: 9-oxa- 3- azabicyclo[4.2.1] nonane hydrochloride (CAS#: 1949816-66-3, ChemSpace) | |
| 22 | 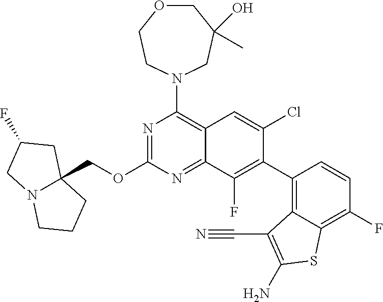 [SMILES: CC1(O)COCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- (6-hydroxy-6- methyl-1,4- oxazepan-4- yl)quinazolin-7- yl)-7- fluorobenzo[6] thiophene-3- carbonitrile Isomer 1 | Bis (2,2.2- trifluoro- aceate) | Step 2: 6- methyl-1,4- oxazepan-6-ol hydrochloride isomer 1 (Intermediate A1) | Chiral separation after Step 2. Details included below. |
| 23 | 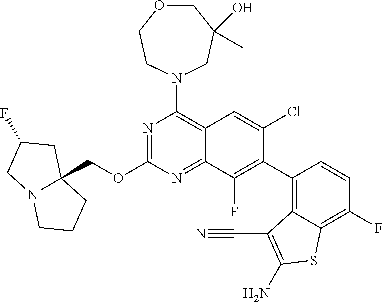 [SMILES: CC1(O)COCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- (6-hydroxy-6- methyl-1,4- oxazepan-4- yl)quinazolin-7- yl)-7- fluorobenzo[b] thiophene-3- carbonitrile Isomer 2 | Bis (2,2,2- trifluoro- aceate) | Step 2: 6- methyl-1,4- oxazepan-6-ol hydrochloride isomer 1 (Intermediate A1) | Chiral separation after Step 2. Details included below. |
| 24 | 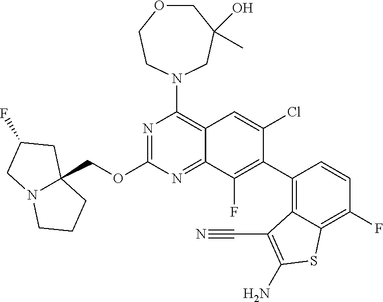 [SMILES: CC1(O)COCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- (6-hydroxy-6- methyl-1,4- oxazepan-4- yl)quinazolin-7- yl)-7- fluorobenzo[b] thiophene-3- carbonitrile Mixture of Isomer 3 and 4 | Bis (2,2,2- trifluoro- aceate) | Step 2: 6- methyl-1,4- oxazepan-6-ol hydrochloride isomer 2 (Intermediate A2) | |
| 25 | 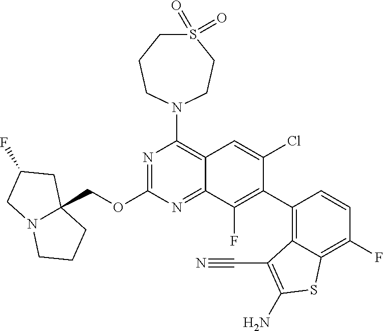 [SMILES: N#Cc1c(N)sc2c(F)ccc(-c3c(Cl)cc4c(N5CCCS(=O)(=O)CC5)nc(OC[C@@]56CCCN5C[C@H](F)C6)nc4c3F)c12] | 2-Amino-4-(6- chloro-4-(1,1- dioxido-1,4- thiazepan-4-yl)- 8-fluoro-2- (((2R,7aS)-2- fluorotetrahydro- 1H-pyrrolizin- 7a(5H)- yl)methoxy) quinazolin-7-yl)-7- fluorobenzo[b] thiophene-3- carbonitrile | Bis (2,2,2- trifluoro- aceate) | Step 2: 1λ⁶,4- thiazepane- 1,1-dione hydrochloride (CAS#: 595597-04-9, Enamine) | |
| 26 | 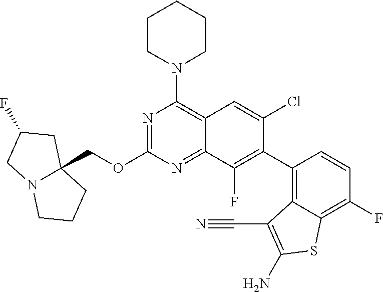 [SMILES: N#Cc1c(N)sc2c(F)ccc(-c3c(Cl)cc4c(N5CCCCC5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (piperidin-1- yl)quinazolin-7- yl)-7- fluorobenzo[6] thiophene-3- carbonitrile | Bis (2,2,2- trifluoro- aceate) | Step 2: piperidine (CAS#: 110- 89-4, Spectrum Chemicals & Laboratory Products) | |
| 27 | 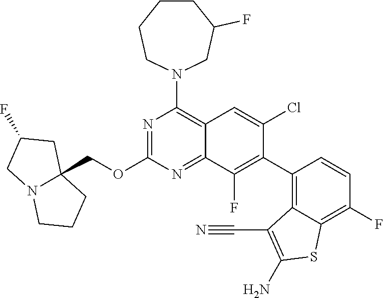 [SMILES: N#Cc1c(N)sc2c(F)ccc(-c3c(Cl)cc4c(N5CCCCC(F)C5)nc(OC[C@@]56CCCN5C[C@H](F)C6)nc4c3F)c12] | 2-Amino-4-(6- chloro-8-fluoro- 4-(3- fluoroazepan-1- yl)-2-(((2R,7aS)- 2- fluorotetrahydro- 1H-pyrrolizin- 7a(5H)- yl)methoxy) quinazolin-7-yl)-7- fluorobenzo[b] thiophene-3- carbonitrile | Bis (2,2,2- trifluoro- aceate) | Step 2: 3- fluoroazepane hydrochloride (CAS#: 18668-74-1, Enamine) | |
| 28 | 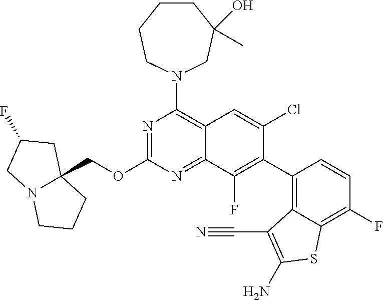 [SMILES: CC1(O)CCCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- (3-hydroxy-3- methylazepan-1- yl)quinazolin-7- yl)-7- fluorobenzo[b] thiophene-3- carbonitrile Isomer 1 | Bis (2,2,2- trifluoro- aceate) | Step 2: 3- methylazepan- 3-ol hydrochloride Isomer 1 (Intermediate E1) | |
| 29 | 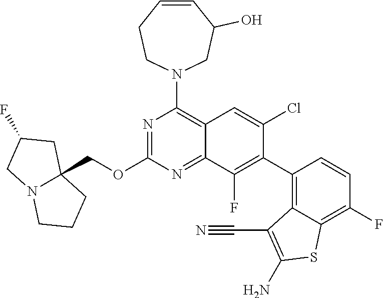 [SMILES: N#Cc1c(N)sc2c(F)ccc(-c3c(Cl)cc4c(N5CCC=CC(O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (3-hydroxy- 2,3,6,7- tetrahydro-1H- azepin-1- yl)quinazolin-7- yl)-7- fluorobenzo[b] thiophene-3- carbonitrile | Bis (2,2,2- trifluoro- aceate) | Step 2: 2,3,6,7- tetrahydro- 1H-azepin-3- ol hydrochloride (Intermediate D) | |
| 30 | 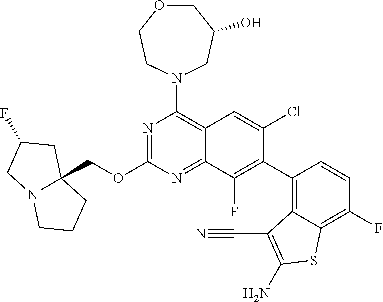 [SMILES: N#Cc1c(N)sc2c(F)ccc(-c3c(Cl)cc4c(N5CCOC[C@H](O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- ((R)-6-hydroxy- 1,4-oxazepan-4- yl)quinazolin-7- yl)-7- fluorobenzo[b] thiophene-3- carbonitrile | Bis (2,2,2- trifluoro- aceate) | Step 2: (R)- [1,4]oxazepan- 6-ol (CAS#: 1022915-33-8, J&W Pharmlab) | |
| 31 | 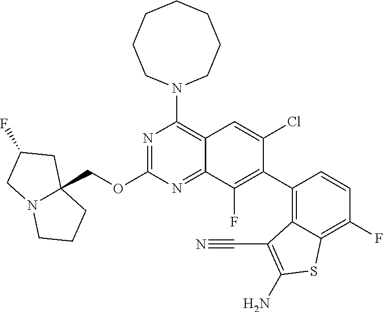 [SMILES: N#Cc1c(N)sc2c(F)ccc(-c3c(Cl)cc4c(N5CCCCCCC5)nc(OC[C@@]56CCCN5C[C@H](F)C6)nc4c3F)c12] | 2-Amino-4-(4- (azocan-1-yl)-6- chloro-8-fluoro- 2-(((2R,7aS)-2- fluorotetrahydro- 1H-pyrrolizin- 7a(5H)- yl)methoxy) quinazolin-7-yl)-7- fluorobenzo[b] thiophene-3- carbonitrile | Bis (2,2,2- trifluoro- aceate) | Step 2: azocane (CAS#: 1121- 92-2, Aldrich) | |
| 32 | 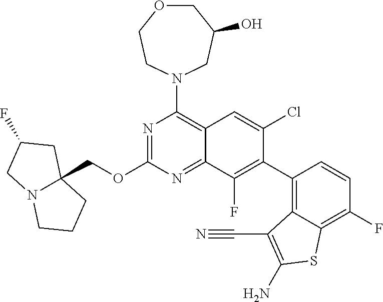 [SMILES: N#Cc1c(N)sc2c(F)ccc(-c3c(Cl)cc4c(N5CCOC[C@@H](O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- ((S)-6-hydroxy- 1,4-oxazepan-4- yl)quinazolin-7- yl)-7- fluorobenzo[b] thiophene-3- carbonitrile | Bis (2,2,2- trifluoro- aceate) | Step 2: (S)- [1,4]oxazepan- 6-ol (CAS#: 1373232-31-5, J&W Pharmlab) | |
| 33 | 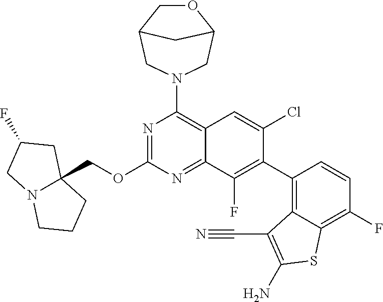 [SMILES: N#Cc1c(N)sc2c(F)ccc(-c3c(Cl)cc4c(N5CC6COC(C6)C5)nc(OC[C@@]56CCCN5C[C@H](F)C6)nc4c3F)c12] | 4-(4-(6-Oxa-3- azabicyclo[3.2.1] octan-3-yl)-6- chloro-8-fluoro- 2-(((2R,7aS)-2- fluorotetrahydro- 1H-pyrrolizin- 7a(5H)- yl)methoxy) quinazolin-7-yl)-2- amino-7- fluorobenzo[b] thiophene-3- carbonitrile | Bis (2,2,2- trifluoro- aceate) | Step 2: 6-oxa- 3- azabicyclo[3.2.1] octane hydrochloride (CAS#: 1461708-41-7, Enamine) | |
| 34 | 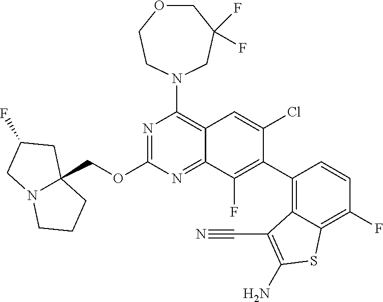 [SMILES: N#Cc1c(N)sc2c(F)ccc(-c3c(Cl)cc4c(N5CCOCC(F)(F)C5)nc(OC[C@@]56CCCN5C[C@H](F)C6)nc4c3F)c12] | 2-Amino-4-(6- chloro-4-(6,6- difluoro-1,4- oxazepan-4-yl)- 8-fluoro-2- (((2R,7aS)-2- fluorotetrahydro- 1H-pyrrolizin- 7a(5H)- yl)methoxy) quinazolin-7-yl)-7- fluorobenzo[b] thiophone-3- carbonitrile | Bis (2,2,2- trifluoro- aceate) | Step 2: 6,6- difluoro-1,4- oxazepane hydrochloride (CAS#: 1341039-23-3, Enamine) | |
| 35 | 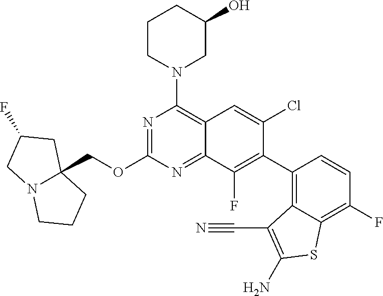 [SMILES: N#Cc1c(N)sc2c(F)ccc(-c3c(Cl)cc4c(N5CCC[C@@H](O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- ((R)-3- hydroxypiperidin- 1-yl)quinazolin- 7-yl)-7- fluorobenzo[b] thiophene-3- carbonitrile | Bis (2,2,2- trifluoro- aceate) | Step 2: (3R)- piperidin-3-ol (CAS#: 62414-68-0, Combi-Blocks Inc.) | |
| 36 | 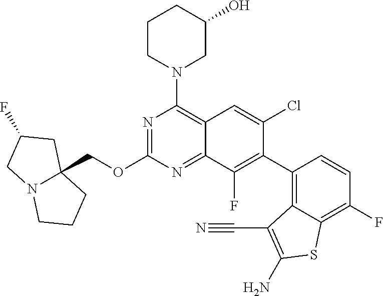 [SMILES: N#Cc1c(N)sc2c(F)ccc(-c3c(Cl)cc4c(N5CCC[C@H](O)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- ((S)-3- hydroxypiperidin- 1-yl)quinazolin- 7-yl)-7- fluorobenzo[b] thiophene-3- carbonitrile | Bis (2,2,2- trifluoro- aceate) | Step 2: (S)-3- hydroxypiperi- dine (CAS#: 24211-55-0, Combi-Blocks Inc.) | |
| 37 | 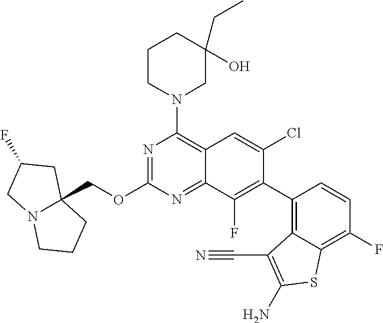 [SMILES: CCC1(O)CCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-4-(3- ethyl-3- hydroxypiperidin- 1-yl)-8-fluoro-2- (((2R,7aS)-2- fluorotetrahydro- 1H-pyrrolizin- 7a(5H)- yl)methoxy) quinazolin-7-yl)-7- fluorobenzo[b] thiophone-3- carbonitrile | Bis (2,2,2- trifluoro- aceate) | Step 2: 3- ethylpiperidin- 3-ol (CAS#: 1177299-74-9, Enamine) | |
| 38 | 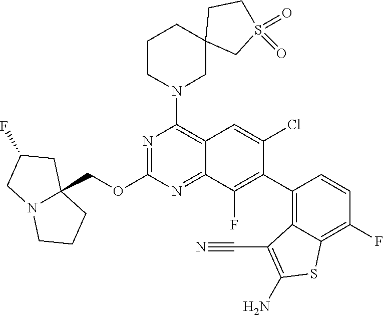 [SMILES: N#Cc1c(N)sc2c(F)ccc(-c3c(Cl)cc4c(N5CCCC6(CCS(=O)(=O)C6)C5)nc(OC[C@@]56CCCN5C[C@H](F)C6)nc4c3F)c12] | 2-Amino-4-(6- chloro-4-(2,2- dioxido-2-thia-7- azaspiro[4.5]decan- 7-yl)-8-fluoro- 2-(((2R,7aS)-2- fluorotetrahydro- 1H-pyrrolizin- 7a(5H)- yl)methoxy) quinazolin-7-yl)-7- fluorobenzo[b] thiophene-3- carbonitrile | Bis (2,2,2- trifluoro- aceate) | Step 2: 2λ6- thia-7- azaspiro[4.5] decane-2,2- dione hydrochloride (CAS#: 1909306-48-4, Enamine) | |
| 39 | 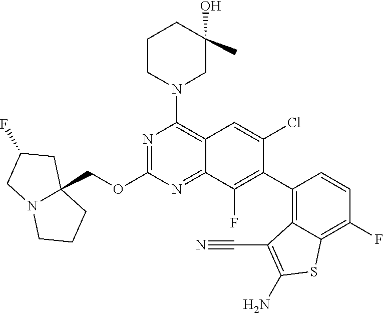 [SMILES: C[C@]1(O)CCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- ((S)-3-hydroxy-3- methylpiperidin- 1-yl)quinazolin- 7-yl)-7- fluorobenzo[b] thiophene-3- carbonitrile | Bis (2,2,2- trifluoro- aceate) | Step 2: (3S)- 3- methylpiperidin- 3-ol hydrochloride (CAS#: 2305080-37-7, eNovation Chemicals LLC) | |
| 40 | 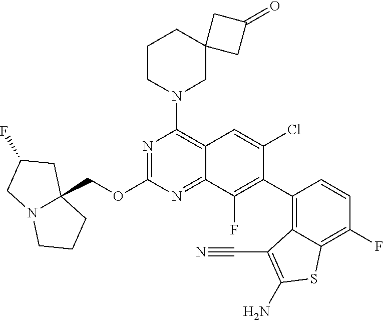 [SMILES: N#Cc1c(N)sc2c(F)ccc(-c3c(Cl)cc4c(N5CCCC6(CC(=O)C6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (2-oxo-6- azaspiro[3.5]nonan- 6-yl)quinazolin-7- yl)-7- fluorobenzo[b] thiophene-3- carbonitrile | Bis (2,2,2- trifluoro- aceate) | Step 2: 6- azaspiro[3.5] nonan-2-one hydrochloride (CAS#: 1359704-57-6, Pharmablock, Inc.) | |
| 41 | 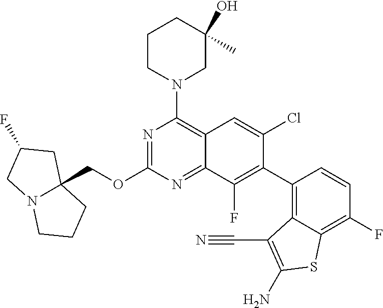 [SMILES: C[C@@]1(O)CCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- ((R)-3-hydroxy- 3- methylpiperidin- 1-yl)quinazolin- 7-yl)-7- fluorobenzo[b] thiophene-3- carbonitrile | Bis (2,2,2- trifluoro- aceate) | Step 2: (3R)- 3- methylpiperidin- 3-ol hydrochloride (CAS#: 2305080-34-4, Pharmablock, Inc.) | |
| 42 | 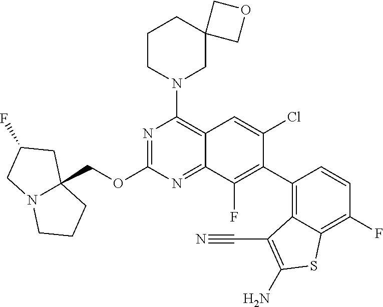 [SMILES: N#Cc1c(N)sc2c(F)ccc(-c3c(Cl)cc4c(N5CCCC6(COC6)C5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (2-oxa-6- azaspiro[3.5] nonan-6- yl)quinazolin-7- yl)-7- fluorobenzo[b] thiophene-3- carbonitrile | Bis (2,2,2- trifluoro- aceate) | Step 2: 2-oxa- 6- azaspiro[3.5] nonane hydrochloride (CAS#: 1414885-19-0, Enamine) | |
| 43 | 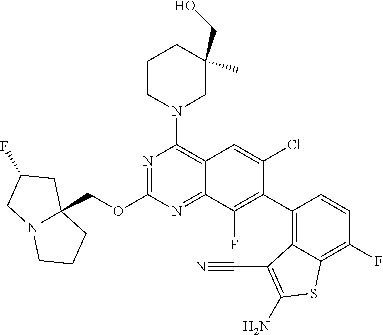 [SMILES: C[C@@]1(CO)CCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-8-fluoro- 2-(((2R,7aS)-2- fluorotetrahydro- 1H-pyrrolizin- 7a(5H)- yl)methoxy)-4- ((R)-3- (hydroxymethyl)- 3- methylpiperidin- 1-yl)quinazolin- 7-yl)-7- fluorobenzo[b] thiophene-3- carbonitrile | Bis (2,2,2- trifluoro- aceate) | Step 1: [(3R)- 3- methylpiperidin- 3- yl]methanol hydrochloride (CAS#: 1956437-13-0, Pharmablock, Inc.) | |
| 44 | 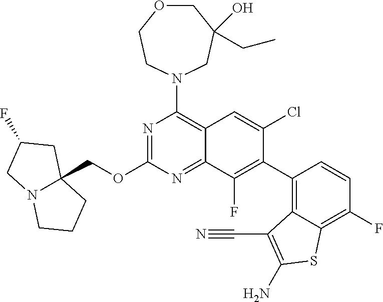 [SMILES: CCC1(O)COCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-4-(6- ethyl-6-hydroxy- 1,4-oxazepan-4- yl)-8-fluoro-2- (((2R,7aS)-2- fluorotetrahydro- 1H-pyrrolizin- 7a(5H)- yl)methoxy) quinazolin-7-yl)-7- fluorobenzo[b] thuophene-3- carbonitrile Isomer 1 | 2,2,2- trifluoro- aceate | Step 2: 6- ethyl-1,4- oxazepan-6-ol hydrochloride Isomer 1 (Intermediate C1) | |
| 45 | 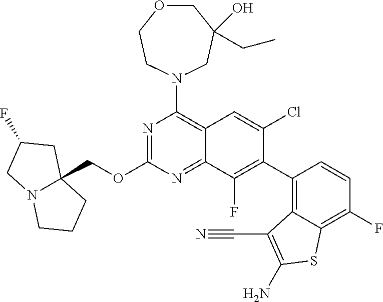 [SMILES: CCC1(O)COCCN(c2nc(OC[C@@]34CCCN3C[C@H](F)C4)nc3c(F)c(-c4ccc(F)c5sc(N)c(C#N)c45)c(Cl)cc23)C1] | 2-Amino-4-(6- chloro-4-(6- ethyl-6-hydroxy- 1,4-oxazepan-4- yl)-8-fluoro-2- (((2R,7aS)-2- fluorotetrahydro- 1H-pyrrolizin- 7a(5H)- yl)methoxy) quinazolin-7-yl)-7- fluorobenzo[b] thiophene-3- carbonitrile Isomer 1 | 2,2,2- trifluoro- aceate | Step 2: 6- ethyl-1,4- oxazepan-6-ol hydrochloride Isomer 1 (Intermediate C2) | |
| 46 | 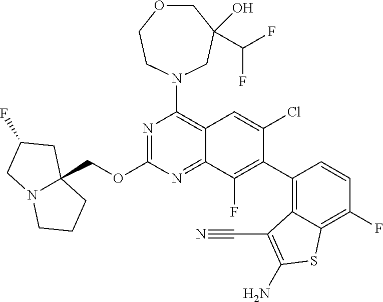 [SMILES: N#Cc1c(N)sc2c(F)ccc(-c3c(Cl)cc4c(N5CCOCC(O)(C(F)F)C5)nc(OC[C@@]56CCCN5C[C@H](F)C6)nc4c3F)c12] | 2-Amino-4-(6- chloro-4-(6- (difluoromethyl)- 6-hydroxy-1,4- oxazepan-4-yl)- 8-fluoro-2- (((2R,7aS)-2- fluorotetrahydro- 1H-pyrrolizin- 7a(5H)- yl)methoxy) quinazolin-7-yl)-7- fluorobenzo[b] thiophone-3- carbonitrile Isomer 1 | bis 2,2,2- trifluoro- aceate | Step 2: 6- (difluoromethyl)- 1,4- oxazepan-6-ol hydrochloride Isomer 1 (Intermediate B1) and tert- butyl (4-(6- chloro-8- fluoro-2- (((2R,7aS)-2- fluorotetrahydro- 1H- pyrrolizin- 7a(5H)- yl)methoxy)- 4-(methylthio) quinazolin-7- | |
| | | | | yl)-3-cyano-7- | |
| | | | | fluorobenzo[b] | |
| | | | | thiophen-2- | |
| | | | | yl)carbamate | |
| | | | | (Intermediate | |
| | | | | F1) | |
| 47 | 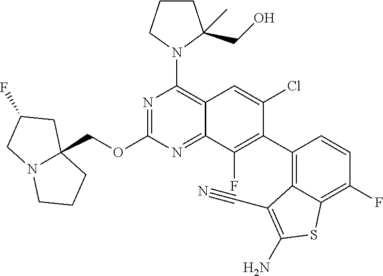 [SMILES: C[C@@]1(CO)CCCN1c1nc(OC[C@@]23CCCN2C[C@H](F)C3)nc2c(F)c(-c3ccc(F)c4sc(N)c(C#N)c34)c(Cl)cc12] | 2-Amino-4-(6- chloro-8-fluoro- 2-(((2R,7aS)-2- fluorotetrahydro- 1H-pyrrolizin- 7a(5H)- yl)methoxy)-4- ((S)-2- (hydroxymethyl)- 2- methylpyrrolidin- 1-yl)quinazolin- 7-yl)-7- fluorobenzo[6] thiophene-3- carbonitrile | 2,2,2- trifluoro- acetate | Step 2: [(2S)- 2-methyl-2- pyrrolidinyl] methanol (CAS#: 115512-58-8. Enamine) | |
| 48 | 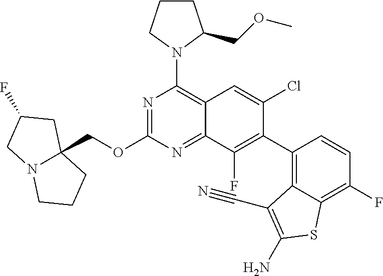 [SMILES: COC[C@@H]1CCCN1c1nc(OC[C@@]23CCCN2C[C@H](F)C3)nc2c(F)c(-c3ccc(F)c4sc(N)c(C#N)c34)c(Cl)cc12] | 2-Amino-4-(6- chloro-8-fluoro- 2-(((2R,7a.S)-2- fluorotetrahydro- 1H-pyrrolizin- 7a(5H)- yl)methoxy)-4- ((S)-2- (methoxymethyl) pyrrolidin-1- yl)quinazolin-7- yl)-7- fluorobenzo[b] thiophene-3- carbonitrile | 2,2,2- trifluoro- acetate ate | Step 2: (2S)- 2- (methoxymethyl) pyrrolidine (CAS#: 63126-47-6, Acros Organics) | |
| 49 | 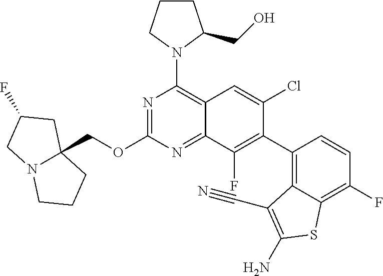 [SMILES: N#Cc1c(N)sc2c(F)ccc(-c3c(Cl)cc4c(N5CCC[C@H]5CO)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- ((S)-2- (hydroxymethyl) pyrrolidin-1- yl)quinazolin-7- yl)-7- fluorobenzo[b] thiophene-3- carbonitrile | 2,2,2- trifluoro- acetate | Step 2: [(2S)- pyrrolidin-2- yl]methanol (CAS#: 23356-96-9, Sigma Aldrich) | |
| 50 | 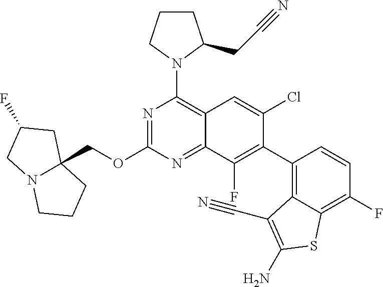 [SMILES: N#CC[C@@H]1CCCN1c1nc(OC[C@@]23CCCN2C[C@H](F)C3)nc2c(F)c(-c3ccc(F)c4sc(N)c(C#N)c34)c(Cl)cc12] | 2-Amino-4-(6- chloro-4-((S)-2- (cyanomethyl) pyrrolidin-1-yl)-8- fluoro-2- (((2R,7aS)-2- fluorotetrahydro- 1H-pyrrolizin- 7a(5H)- yl)methoxy) quinazolin-7-yl)-7- fluorobenzo[b] thiophene-3- carbonitrile | 2,2,2- trifluoro- acetate | Step 2: (S)-2- (Pyrrolidin-2- yl)acetonitrile (CAS#: 1186242-37-4, Enamine) | |
| 51 | 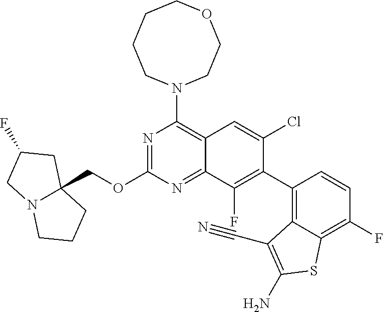 [SMILES: N#Cc1c(N)sc2c(F)ccc(-c3c(Cl)cc4c(N5CCCCOCC5)nc(OC[C@@]56CCCN5C[C@H](F)C6)nc4c3F)c12] | 2-Amino-4-(6- chloro-8-fluoro- 2-(((2R,7aS)-2- fluorotetrahydro- 1H-pyrrolizin- 7a(5H)- yl)methoxy)-4- (1,4-oxazocan-4- yl)quinazolin-7- yl)-7- fluorobenzo[b] thiophene-3- carbonitrile | Bis(2,2,2- trifluoro- acetate) | Step 2: 1,4- oxazocane (CAS#: 4741- 36-0, Princeton BioMolecular Research, Inc) | |
| 52 | 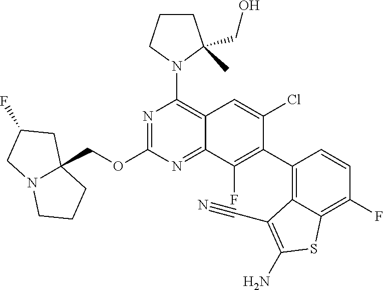 [SMILES: C[C@]1(CO)CCCN1c1nc(OC[C@@]23CCCN2C[C@H](F)C3)nc2c(F)c(-c3ccc(F)c4sc(N)c(C#N)c34)c(Cl)cc12] | 2-Amino-4-(6- chloro-8-fluoro- 2-(((2R,7aS)-2- fluorotetrahydro- 1H-pyrrolizin- 7a(5H)- yl)methoxy)-4- ((R)-2- (hydroxymethyl)- 2- methylpyrrolidin- 1-yl)quinazolin- 7-yl)-7- fluorobenzo[b] thiophene-3- carbonitrile | 2,2,2- trifluoro- acetate | Step 2: (2R)- 2-methyl- pyrrolidine-2- methanol hydrochloride (CAS#: 1408057-43-1, Combi Blocks Inc) | |
| 53 | 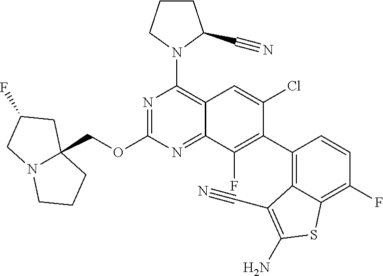 [SMILES: N#Cc1c(N)sc2c(F)ccc(-c3c(Cl)cc4c(N5CCC[C@H]5C#N)nc(OC[C@@]56CCCN5C[C@H](F)C6)nc4c3F)c12] | (2S)-1-(7-(2- Amino-3-cyano- 7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)pyrrolidine-2- carbonitrile | Bis(2, 2,2- trifluoro- acetate) | Step 2: (2S)- 2- pyrrolidine- carbonitrile hydrochloride (CAS#: 65732-69-6, Oakwood Products Inc) | |
| 54 | 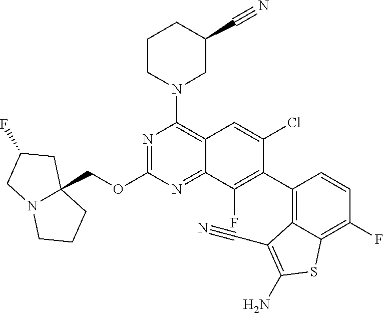 [SMILES: N#Cc1c(N)sc2c(F)ccc(-c3c(Cl)cc4c(N5CCC[C@@H](C#N)C5)nc(OC[C@@]56CCCN5C[C@H](F)C6)nc4c3F)c12] | (3R)-1-(7-(2- Amino-3-cyano- 7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)piperidine-3- carbonitrile | 2,2,2- trifluoro- acetate | Step 2: (3R)- 3- piperidine- carbonitrile (CAS#: 1159736-73-8, Aurum Pharmatech LLC) | |
| 65 | 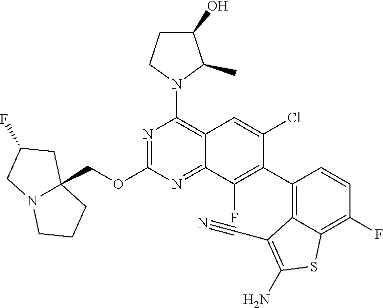 [SMILES: C[C@@H]1[C@H](O)CCN1c1nc(OC[C@@]23CCCN2C[C@H](F)C3)nc2c(F)c(-c3ccc(F)c4sc(N)c(C#N)c34)c(Cl)cc12] | 2-Amino-4-(6- chloro-8-fluoro- 2-(((2R,7aS)-2- fluorotetrahydro- 1H-pyrrolizin- 7a(5H)- yl)methoxy)-4- ((2R,3R)-3- hydroxy-2- methylpyrrolidin- 1-yl)quinazolin- 7-yl)-7- fluorobenzo[b] thiophene-3- carbonitrile | Bis (2,2,2- trifluoro- aceate) | Step 2: (2R, 3R)-2-methyl- pyrolidin-3-ol hydrochloride (CAS#: 1107659-80-2, J&W Pharmlab, LLC) | |
| 66 | 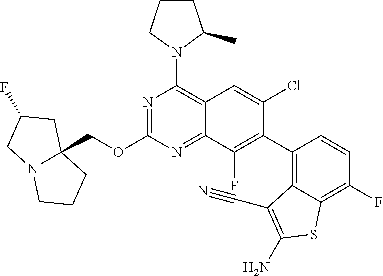 [SMILES: C[C@@H]1CCCN1c1nc(OC[C@@]23CCCN2C[C@H](F)C3)nc2c(F)c(-c3ccc(F)c4sc(N)c(C#N)c34)c(Cl)cc12] | 2-Amino-4-(6- chloro-8-fluoro- 2-(((2R,7aS)-2- fluorotetrahydro- 1H-pyrrolizin- 7a(5H)- yl)methoxy)-4- ((R)-2- methylpyrrolidin- 1-yl)quinazolin- 7-yl)-7- fluorobenzo[b] thiophene-3- carbonitrile | Bis (2,2,2- trifluoro- aceate) | Step 2: (R)-2- methyl- pyrrolidine (CAS#: 41720-98-3, Combi- Blocks) | |
| 67 | 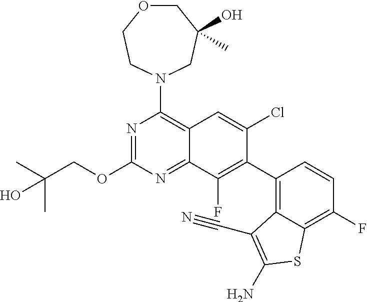 [SMILES: CC(C)(O)COc1nc(N2CCOC[C@@](C)(O)C2)c2cc(Cl)c(-c3ccc(F)c4sc(N)c(C#N)c34)c(F)c2n1] | 2-Amino-4-(6- chloro-8-fluoro- 2-(2-hydroxy-2- methylpropoxy)- 4-((S)-6-hydroxy- 6-methyl-1,4- oxazepan-4- yl)quinazolin-7- yl)-7- fluorobenzo[6] thiophene-3- carbonitrile | Mono (2,2,2- trifluoro- aceate | Step 2: 6- methyl-1,4- oxazepan-6-ol hydrochloride isomer 2 (Intermediate A2) | |

TABLE 3
Conditions for Chiral SFC Separation.

| Separation | SFC Conditions | Peak to Ex# |
|---|---|---|
| 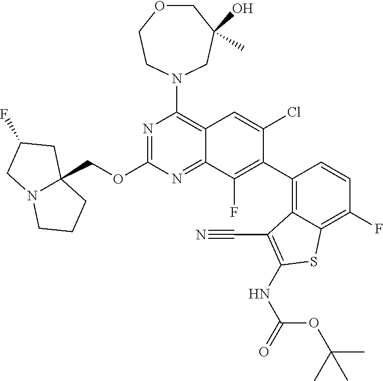 [SMILES: CC(C)(C)OC(=O)Nc1sc2c(F)ccc(-c3c(Cl)cc4c(N5CCOC[C@@](C)(O)C5)nc(OC[C@@]56CCCN5C[C@H](F)C6)nc4c3F)c2c1C#N] | Column: ChiralPak AD (2 x 25 cm, 5 μm). Mobile phase: 45% MeOH with 0.2% DEA. Flowrate: 80 mL/min. Yield: 246 mg sample was submitted to generate 47 mg of peak 1 with an ee of 100% and 64 mg of peak 2 with an ee of 95%. | Peak 1: Example 22 Peak 2: Example 23 |

TABLE 4
Analytical Data for Examples 2 to 54, 65 to 67.

| | MS | |
|---|---|---|
| Ex. | m/z (ESI): | |
| # | (M + H)⁺ | ¹H NMR |
| 2 | 669.7 | ¹H NMR (400 MHz, METHANOL-d₄) δ ppm 7.97-8.03 (m, 1 H), |
| | | 7.21-7.28 (m, 1 H), 7.03-7.11 (m, 1 H), 5.46-5.67 (m, 1 H), |
| | | 4.56-4.74 (m, 2 H), 4.04-4.15 (m, 2 H), 3.97-4.03 (m, 1 H), |
| | | 3.80-3.97 (m, 6 H), 3.67-3.75 (m, 1 H), 3.48 (br d, J = 8.6 Hz, 2 |
| | | H), 2.53-2.78 (m, 2 H), 2.30-2.49 (m, 3 H), 2.07-2.25 (m, 1 H), |
| | | 1.75-1.99 (m, 6 H). |
| 3 | 652.8 | ¹H NMR (400 MHz, METHANOL-d₄) δ ppm 8.09-8.16 (m, 1 H), |
| | | 7.19-7.29 (m, 1 H), 7.03-7.12 (m, 1 H), 5.44-5.67 (m, 1 H), |
| | | 4.69 (s, 2 H), 3.97-4.30 (m, 5 H), 3.80-3.97 (m, 2 H), 3.42- |
| | | 3.55 (m, 1 H), 3.10-3.23 (m, 1 H), 2.58 (s, 2 H), 2.24-2.50 (m, 6 |
| | | H), 2.08-2.23 (m, 2 H), 1,93-2.08 (m, 2 H). |
| 4 | 643.8 | ¹H NMR (400 MHz, METHANOL-d₄) δ ppm 8.23-8.32 (m, 1 H), |
| | | 7.20-7.29 (m, 1 H), 7.02-7.13 (m, 1 H), 5.46-5.67 (m, 1 H), |
| | | 4.58-4.75 (m, 2 H), 4.31-4.43 (m, 1 H), 4.08-4.25 (m, 3 H), |
| | | 3.77-4.07 (m, 4 H), 3.41-3.56 (m, 1 H), 2.53-2.79 (m, 2 H), |
| | | 2.28-2.50 (m, 3 H), 1.99-2.24 (m, 4 H), 1.87-1.98 (m, 1 H), |
| | | 1.63-1.77 (m, 1 H), 1.42-1.58 (m, 1 H). |
| 5 | 642.9 | ¹H NMR (400 MHz, METHANOL-d₄) δ ppm 8.24-8.32 (m, 1 H), |
| | | 7.19-7.30 (m, 1 H), 7.07 (s, 1 H), 5.45-5.67 (m, 1 H), 4.58- |
| | | 4.76 (m, 2 H), 4.31-4.43 (m, 1 H), 4.07-4.25 (m, 3 H), 3.78- |
| | | 4.07 (m, 4 H), 3.41-3.56 (m, 1 H), 2.53-2.79 (m, 2 H), 2.28- |
| | | 2.52 (m, 3 H), 2.00-2.25 (m, 4 H), 1.83-1.99 (m, 1 H), 1.64- |
| | | 1.78 (m, 1 H), 1.43-1.57 (m, 1 H). |
| 6 | 643.8 | ¹H NMR (400 MHz, METHANOL-d₄) δ ppm 8.13-8.19 (m, 1 H), |
| | | 7.19-7.28 (m, 1 H), 7.02-7.12 (m, 1 H), 5.46-5.68 (m, 1 H), |
| | | 4.39-4.66 (m, 5 H), 4.18-4.26 (m, 2 H), 3.83-4.13 (m, 6 H), |
| | | 3.45-3.55 (m, 1 H), 2.52-2.78 (m, 2 H), 2.30-2.48 (m, 3 H), |
| | | 2.10-2.25 (m, 1 H). |
| 7 | 654.0 | ¹H NMR (400 MHz, METHANOL-d₄) δ ppm 8.26-8.40 (m, 1 H), |
| | | 7.19-7.27 (m, 1 H), 7.03-7.11 (m, 1 H), 5.45-5.68 (m, 1 H), |
| | | 4.79-4.91 (m, 2 H), 4.53-4.76 (m, 3 H), 4.31-4.43 (m, 1 H), |
| | | 3.80-4.12 (m, 3 H), 3.48 (br s, 3 H), 2.52-2.83 (m, 2 H), 2.06- |
| | | 2.51 (m, 7 H), 1.78-1.95 (m, 2 H), 1.36-1.49 (m, 1 H). |
| 8 | 641.8 | ¹H NMR (400 MHz, METHANOL-d₄) δ ppm 8.15-8.21 (m, 1 H), |
| | | 7.20-7.30 (m, 1 H), 7.02-7.12 (m, 1 H), 5.47-5.68 (m, 1 H), |
| | | 4.51-4.69 (m, 4 H), 4.25-4.37 (m, 2 H), 3.81-4.11 (m, 3 H), |
| | | 3.42-3.55 (m, 1 H), 2.52-2.77 (m, 4 H), 2.29-2.48 (m, 3 H), |
| | | 2.05-2.23 (m, 3 H), 1.86-1.96 (m, 2 H). |
| 9 | 702.7 | ¹H NMR (400 MHz, METHANOL-d₄) δ ppm 8.20-8.29 (m, 1 H), |
| | | 7.20-7.29 (m, 1 H), 7.07 (s, 1 H), 5.43-5.67 (m, 1 H), 4.70 (s, 3 |
| | | H), 3.79-4.34 (m, 6 H), 3.41-3.54 (m, 1 H), 3.21-3.30 (m, 2 H), |
| | | 2.80-2.92 (m, 1 H), 2.52-2.78 (m, 2 H), 2.30-2.51 (m, 6 H), |
| | | 2.06-2.29 (m, 3 H). |
| 10 | 703.0 | ¹H NMR (400 MHz, METHANOL-d₄) δ ppm 7.96-8.10 (m, 1 H), |
| | | 7.24 (s, 1 H), 7.03-7.12 (m, 1 H), 5.46-5.67 (m, 1 H), 4.88 (br d, |
| | | J = 13.8 Hz, 1 H), 4.60-4.74 (m, 2 H), 4.24-4.44 (m, 1 H), 3.93 |
| | | (s, 6 H), 3.40-3.54 (m, 2 H), 2.67 (s, 3 H), 2.28-2.51 (m, 3 H), |
| | | 1.89-2.24 (m, 6 H). |
| 11 | 670.0 | ¹H NMR (400 MHz, METHANOL-d₄) δ ppm 8.08-8.17 (m, 1 H), |
| | | 7.20-7.28 (m, 1 H), 7.01-7.13 (m, 1 H), 5.45-5.68 (m, 1 H), |
| | | 4.52-4.76 (m, 3 H), 3.79-4.38 (m, 4 H), 3.52 (br d, J = 13.4 Hz, 3 |
| | | H), 2.52-2.82 (m, 2 H), 2.25-2.51 (m, 4 H), 2.04-2.24 (m, 1 H), |
| | | 1.84-2.00 (m, 2 H), 1.70-1.82 (m, 1 H), 1.27 (d, J = 11.1 Hz, 3 |
| | | H), |
| 12 | 691.8 | ¹H NMR (400 MHz, METHANOL-d₄) δ ppm 7.98-8.04 (m, 1 H), |
| | | 7.20-7.28 (m, 1 H), 7.02-7.12 (m, 1 H), 5.48-5.68 (m, 1 H), |
| | | 4.85-4.99 (m, 1 H), 4.76 (br s, 2 H), 4.39-4.54 (m, 1 H), 3.82- |
| | | 4.12 (m, 3 H), 3.51 (br d, J = 6.5 Hz, 4 H), 2.53-2.81 (m, 2 H), |
| | | 2.29-2.49 (m, 4 H), 2.11-2.25 (m, 1 H), 1.96-2.11 (m, 2 H), |
| | | 1.69-1.88 (m, 1 H). |
| 13 | 667.8 | ¹H NMR (400 MHz, METHANOL-d₄) δ ppm 7.98-8.05 (m, 1 H), |
| | | 7.21-7.28 (m, 1 H), 7.03-7.11 (m, 1 H), 5.48-5.67 (m, 1 H), |
| | | 4.57-4.75 (m, 2 H), 4.07-4.27 (m, 2 H), 3.82-4.07 (m, 4 H), |
| | | 3.71-3.82 (m, 1 H), 3.43-3.55 (m, 1 H), 2.77 (s, 4 H), 2.30- |
| | | 2.51 (m, 3 H), 1.95 (s, 5 H). |
| 14 | 732.0 | ¹H NMR (400 MHz, METHANOL-d₄) δ ppm 7.95-8.01 (m, 1 H), |
| | | 7.21-7.28 (m, 1 H), 7.03-7.11 (m, 1 H), 5.47-5.68 (m, 1 H), |
| | | 4.61-4.76 (m, 2 H), 4.15-4.33 (m, 2 H), 3.81-4.13 (m, 7 H), |
| | | 3.72 (d, J = 7.9 Hz, 2 H), 3.43-3.54 (m, 1 H), 2.99 (s, 3 H), 2.57- |
| | | 2.83 (m, 2 H), 2.45-2.57 (m, 1 H), 2.30-2.41 (m, 2 H), 2.14- |
| | | 2.29 (m, 1 H), 1.97-2.07 (m, 2 H), 1.73-1.84 (m, 2 H). |
| 15 | 706.0 | ¹H NMR (400 MHz, METHANOL-d₄) δ ppm 8.19 (s, 1 H), 7.23 |
| | | (s, 1 H), 7.08 (d, J = 9.4 Hz, 1 H), 5.47-5.69 (m, 1 H), 4.90-5.01 |
| | | (m, 1 H), 4.79-4.88 (m, 2 H), 4.12-4.32 (m, 2 H), 3.72-4.11 |
| | | (m, 5 H), 3.42-3.55 (m, 1 H), 2.44-2.85 (m, 3 H), 1.98-2.43 |
| | | (m, 7 H), 1.82-1.95 (m, 1 H), 1.36-1.57 (m, 1 H). |
| 16 | 641.0 | ¹H NMR (400 MHz, METHANOL-d4) 8 ppm 8.00-8.14 (m, 1 H), |
| | | 7.20-7.28 (m, 1 H), 7.02-7.12 (m, 1 H), 5.46-5.67 (m, 1 H), |
| | | 4.69 (s, 2 H), 4.24-4.36 (m, 3 H), 3.80-4.16 (m, 4 H), 3.41- |
| | | 3.56 (m, 1 H), 2.94-3.02 (m, 2 H), 2.54-2.82 (m, 4 H), 2.29- |
| | | 2.52 (m, 3 H), 2.03-2.28 (m, 3 H). |
| 17 | 648.1 | ¹H NMR (400 MHz, METHANOL-d₄) δ ppm 7.99 (s, 1 H), 7.21- |
| | | 7.28 (m, 1 H), 7.07 (t, J = 8.9 Hz, 1 H), 5.48-5.67 (m, 1 H), 4.55- |
| | | 4.73 (m, 3 H), 4.31-4.46 (m, 1 H), 3.82-4.13 (m, 3 H), 3.58- |
| | | 3.77 (m, 2 H), 3.40-3.54 (m, 3 H), 2.53-2.82 (m, 2 H), 2.30- |
| | | 2.50 (m, 3 H), 1.99-2.27 (m, 4 H), 1.83-1.96 (m, 1 H). |
| 18 | 642.7 | ¹H NMR (400 MHz, METHANOL-d₄) δ ppm 8.19 (d, J = 1.7 Hz, 1 |
| | | H), 7.25 (dd, J = 8.4, 5.0 Hz, 1 H), 7.07 (dd, J = 9.3, 8.5 Hz, 1 H), |
| | | 5.43-5.68 (m, 1 H), 4.70-4.77 (m, 2 H), 4.60 (d, J = 12.8 Hz, 1 |
| | | H), 4.25 (t, J = 5.3 Hz, 4 H), 3.86-4.14 (m, 6 H), 3.43-3.51 (m, 1 |
| | | H), 2.56-2.72 (m, 2 H), 2.30-2.49 (m, 3 H), 2.07-2.25 (m, 5 H). |
| 19 | 642.7 | ¹H NMR (400 MHz, METHANOL-d₄) δ ppm 8.19 (d, J = 1.7 Hz, 1 |
| | | H), 7.25 (dd, J = 8.4, 5.0 Hz, 1 H), 7.07 (dd, J = 9.4, 8.4 Hz, 1 H), |
| | | 5.61 (t, J = 3,4 Hz, 1 H), 5.48 (t, J = 3.2 Hz, 1 H), 4.65-4.75 (m, 2 |
| | | H), 4.25 (t, J-5.3 Hz, 4 H), 3.85-4.01 (m, 6 H), 3.45-3.52 (m, 1 |
| | | H), 2.51-2.80 (m, 2 H), 2.31-2.44 (m, 3 H), 2.12-2.25 (m, 5 H). |
| 20 | 655.0 | ¹H NMR (400 MHz, METHANOL-d₄) δ ppm 8.31 (d, J = 6.8 Hz, 1 |
| | | H), 7.26 (dtd, 1 H), 7.08 (t, J = 8.9 Hz, 1 H), 5.46-5.68 (m, 1 H), |
| | | 4.49-4.74 (m, 5 H), 4.26-4.43 (m, 1 H), 3.82-4.09 (m, 3 H), |
| | | 3.63-3.78 (m, 1 H), 3.35-3.53 (m, 2 H), 2.58-2.81 (m, 2 H), |
| | | 2.27-2.55 (m, 6 H), 2.09-2.23 (m, 1 H), 1.88-2.08 (m, 2 H), |
| | | 1.82 (br dd, J = 8.8, 4.0 Hz, 1 H). |
| 21 | 655.0 | ¹H NMR (400 MHz, METHANOL-d₄) δ ppm 8.01-8.05 (m, 1 H), |
| | | 7.23 (dt, J = 8.4, 5.4 Hz, 1 H), 7.05 (t, J = 9,0 Hz, 1 H), 5.28-5.52 |
| | | (m, 1 H), 4.56-4.74 (m, 3 H), 4.30-4.47 (m, 2 H), 4.16-4.23 |
| | | (m, 1 H), 3.83 (ddd, J = 14.8, 10.6, 4.5 Hz, 1 H), 3.47-3.63 (m, 2 |
| | | H), 3.35-3.46 (m, 3 H), 3.12-3.20 (m, 1 H), 2.30-2.50 (m, 3 H), |
| | | 2.03-2.25 (m, 6 H), 1.67-1.84 (m, 2 H). |
| 22 | 658.8 | ¹H NMR (400 MHz, METHANOL-d₄) δ ppm 8.56 (d, J = 1.5 Hz, 1 |
| | | H), 7.24 (t, J = 6.3 Hz, 1 H), 7.07 (t, J = 8.8 Hz, 1 H), 5.44-5,65 (m, |
| | | 1 H), 4.57-4.74 (m, 2 H), 4.42-4.55 (m, 2 H), 4.09-4.18 (m, 1 |
| | | H), 3.83-4.08 (m, 6 H), 3.63-3.74 (m, 2 H), 3.41-3.58 (m, 1 H), |
| | | 2.55-2.73 (m, 2 H), 2.30-2.51 (m, 3 H), 2.08-2.23 (m, 1 H), |
| | | 1.29 (s, 3 H). |
| 23 | 658.8 | ¹H NMR (400 MHz, METHANOL-d₄) δ ppm 8.56 (d, J = 1.5 Hz, 1 |
| | | H), 7.25 (dd, J = 8,4, 5.2 Hz, 1 H), 7.07 (dd, J = 9.4, 8.4 Hz, 1 H), |
| | | 5.45-5.70 (m, 1 H), 4.62-4.74 (m, 2 H), 4.40-4.57 (m, 2 H), |
| | | 4.08-4.15 (m, 1 H), 3.85-4.06 (m, 6 H), 3.63-3.75 (m, 2 H), |
| | | 3.42-3.59 (m, 1 H), 2.52-2.77 (m, 2 H), 2.31-2.49 (m, 3 H), |
| | | 2.12-2.26 (m, 1 H), 1.29 (s, 3 H). |
| 24 | 658.8 | ¹H NMR (400 MHz, METHANOL-d₄) δ ppm 8.54-8.57 (m, 1 H), |
| | | 7.24 (ddd, J = 8.4, 5.0, 0.8 Hz, 1 H), 7.07 (t, J = 8.9 Hz, 1 H), 5.54- |
| | | 5.66 (m, 1 H), 4.56-4.74 (m, 2 H), 4.42-4.55 (m, 2 H), 3.83- |
| | | 4.16 (m, 7 H), 3.64-3.74 (m, 2 H), 3.44-3.53 (m, 1 H), 2.55- |
| | | 2.77 (m, 2 H), 2.31-2.49 (m, 3 H), 2.15 (br d, J = 10.7 Hz, 1 H), |
| | | 1.29 (s, 3 H). |
| 25 | 677.0 | ¹H NMR (400 MHz, METHANOL-d₄) δ ppm 8.14 (d, J = 1.7 Hz, 1 |
| | | H), 7.25 (ddd, J = 8.4, 5.1, 1.1 Hz, 1 H), 7.08 (dd, J = 9.4, 8.4 Hz, 1 |
| | | H), 5.45-5.68 (m, 1 H), 4.55-4.75 (m, 2 H), 4.31-4.42 (m, 4 H), |
| | | 3.83-4.09 (m, 3 H), 3.65-3.77 (m, 2 H), 3.44-3.53 (m, 1 H), |
| | | 3.34-3.40 (m, 2 H), 2.57-2.79 (m, 2 H), 2.32-2.55 (m, 5 H), |
| | | 2.08-2.23 (m, 1 H). |
| 26 | 613.2 | ¹H NMR (400 MHz, METHANOL-d₄) δ ppm 7.94 (d, J = 1.3 Hz, 1 |
| | | H), 7.19-7.27 (m, 1 H), 7.04-7.10 (m, 1 H), 5.44-5.68 (m, 1 H), |
| | | 4.58-4.75 (m, 2 H), 3.82-4.09 (m, 7 H), 3.43-3.54 (m, 1 H), |
| | | 2.54-2.78 (m, 2 H), 2.31-2.50 (m, 3 H), 2.08-2.24 (m, 1 H), |
| | | 1.65-1.93 (m, 6 H). |
| 27 | 645.0 | ¹H NMR (400 MHz, METHANOL-d₄) δ ppm 8.20 (d, J = 2.1 Hz, 1 |
| | | H), 7.25 (t, J-6.2 Hz, 1 H), 7.07 (dd, J = 9.4, 8,6 Hz, 1 H), 5.42- |
| | | 5.68 (m, 1 H), 5.00-5.25 (m, 1 H), 4.58-4.75 (m, 2 H), 4.18- |
| | | 4.43 (m, 3 H), 3.82-4.09 (m, 4 H), 3.44-3.53 (m, 1 H), 2.54- |
| | | 2.78 (m, 2 H), 2.31-2.49 (m, 3 H), 1.93-2.22 (m, 6 H), 1.56- |
| | | 1.66 (m, 1 H). |
| 28 | 659.0 | ¹H NMR (400 MHz, METHANOL-d₄) δ ppm 8.55 (d, J = 1.5 Hz, 1 |
| | | H), 7.21-7.27 (m, 1 H), 7.05-7.11 (m, 1 H), 5.46-5.69 (m, 1 H), |
| | | 4.58-4.75 (m, 2 H), 4.42-4.55 (m, 2 H), 3.83-4.18 (m, 7 H), |
| | | 3.64-3.74 (m, 2 H), 3.44-3.55 (m, 1 H), 2.55-2.76 (m, 2 H), |
| | | 2.31-2.50 (m, 3 H), 2.08-2.23 (m, 1 H), 1.29 (s, 3 H). |
| 29 | 641.0 | ¹H NMR (400 MHz, METHANOL-d₄) δ ppm 8.23-8.31 (m, 1 H), |
| | | 7.25 (t, J = 6.3 Hz, 1 H), 7.07 (t, J = 9.0 Hz, 1 H), 5.78-5.90 (m, 1 |
| | | H). 5.46-5.70 (m, 1 H), 4.59-4.75 (m, 2 H), 4.32-4.57 (m, 1 H), |
| | | 4.03-4.26 (m, 3 H), 3.81-3.96 (m, 3 H), 3.43-3.53 (m, 1 H), |
| | | 2.54-2.80 (m, 3 H), 2.31-2.51 (m, 3 H), 1.93-2.21 (m, 3 H), |
| | | 1.42-1.77 (m, 1 H). |
| 30 | 645.0 | ¹H NMR (400 MHz, METHANOL-d₄) δ ppm 8.34 (t, J = 2.1 Hz, 1 |
| | | H), 7.24 (ddd, J = 8.3, 5.1, 2.9 Hz, 1 H), 7.07 (dd, J = 9.4, 8.6 Hz, 1 |
| | | H), 5.42-5.68 (m, 1 H), 4.59-4.75 (m, 2 H), 4.31-4.39 (m, 1 H), |
| | | 4.19-4.26 (m, 3 H), 3.89-4.17 (m, 7 H), 3.70-3.77 (m, 1 H), |
| | | 3.44-3.53 (m, 1 H), 2.54-2.76 (m, 2 H), 2.31-2.49 (m, 3 H), |
| | | 2.10-2.23 (m, 1 H). |
| 31 | 641.0 | ¹H NMR (400 MHz, METHANOL-d₄) δ ppm 8.12 (d, J = 1.7 Hz, 1 |
| | | H), 7.26 (ddd, J = 8.4, 5.0, 1.5 Hz, 1 H), 7.08 (t, J = 8.9 Hz, 1 H), |
| | | 5.44-5.71 (m, 1 H), 4.56-4.75 (m, 2 H), 3.98-4.15 (m, 5 H), |
| | | 3.81-3.96 (m, 2 H), 3.43-3.52 (m, 1 H), 2.54-2.73 (m, 2 H), |
| | | 2.31-2.49 (m, 3 H), 2.02-2.20 (m, 5 H), 1.76 (br d, J = 4.2 Hz, 4 |
| | | H), 1.63-1.72 (m, 2 H). |
| 32 | 645.0 | ¹H NMR (400 MHz, METHANOL-d₄) δ ppm 8.34 (dd, J = 3,2, 1.8 |
| | | Hz, 1 H), 7.24 (dd, J = 8.5, 5.1 Hz, 1 H), 7.07 (t, J = 8.9 Hz, 1 H), |
| | | 5.44-5.68 (m, 1 H), 4.58-4.73 (m, 2 H), 4.29-4.47 (m, 1 H), |
| | | 4.19-4.26 (m, 3 H), 3.98-4.17 (m, 4 H), 3.85-3.97 (m, 3 H), |
| | | 3.73 (ddd, J = 12.7, 6.0, 3.9 Hz, 1 H), 3.44-3.55 (m, 1 H), 2.54- |
| | | 2.77 (m, 2 H), 2.31-2.50 (m, 3 H), 2.08-2.23 (m, 1 H). |
| 33 | 641.0 | ¹H NMR (400 MHz, METHANOL-d₄) δ ppm 8.08 (dd, J = 9.4, 1.7 |
| | | Hz, 1 H), 7.21-7.27 (m, 1 H), 7.07 (ddd, J-9.5, 8.4, 2.0 Hz, 1 H), |
| | | 5.42-5.66 (m, 1 H), 4.88 (br d, J = 13.0 Hz, 1 H), 4.52-4.73 (m, 3 |
| | | H), 4.43-4.50 (m, 1 H), 3.82-4.06 (m, 5 H), 3.58-3.78 (m, 2 H), |
| | | 3.44-3.53 (m, 1 H), 2.54-2.77 (m, 3 H), 2.31-2.50 (m, 3 H), |
| | | 2.05-2.21 (m, 3 H). |
| 34 | 665.0 | ¹H NMR (400 MHz, METHANOL-d₄) δ ppm 8.10 (s, 1 H), 7.25 |
| | | (dd, J = 8.4, 5.0 Hz, 1 H), 7.07 (dd, J = 9.4, 8.4 Hz, 1 H), 5.46-5.71 |
| | | (m, 1 H), 4.50-4.73 (m, 4 H), 3.99-4.26 (m, 7 H), 3.84-3.96 |
| | | (m, 2 H), 3.45-3.54 (m, 1 H), 2.55-2.79 (m, 2 H), 2.31-2.50 |
| | | (m, 3 H), 2.10-2.26 (m, 1 H). |
| 35 | 629.0 | ¹H NMR (400 MHz, METHANOL-d₄) δ ppm 8.12 (t, J = 2.0 Hz, 1 |
| | | H), 7.24 (ddd, J = 8.3, 5.1, 2.9 Hz, 1 H), 7.07 (dd, J = 9.3, 8.5 Hz, 1 |
| | | H), 5.41-5.69 (m, 1 H), 4.58-4.75 (m, 2 H), 4.16 (dd, J = 13.1, |
| | | 3.0 Hz, 1 H), 3.75-4.02 (m, 7 H), 3.44-3.53 (m, 1 H), 2.54-2.82 |
| | | (m, 2 H), 2.31-2.49 (m, 3 H), 2.03-2.23 (m, 3 H), 1.77 (br dd, |
| | | J = 9.1, 6.4 Hz, 2 H). |
| 36 | 629.0 | ¹H NMR (400 MHz, METHANOL-d₄) δ ppm 8.11 (s, 1 H), 7.24 |
| | | (dd, J = 8.4, 5.2 Hz, 1 H), 7.07 (dd, J = 9.3, 8.5 Hz, 1 H), 5.45-5.67 |
| | | (m, 1 H), 4.56-4.74 (m, 2 H), 4.16 (br d, J = 12.8 Hz, 1 H), 3.74- |
| | | 4.01 (m, 7 H), 3.44-3.53 (m, 1 H), 2.55-2.77 (m, 2 H), 2.31- |
| | | 2.49 (m, 3 H), 2.03-2.23 (m, 3 H), 1.70-1.82 (m, 2 H). |
| 37 | 657.0 | ¹H NMR (400 MHz, METHANOL-d₄) δ ppm 8.25-8.28 (m, 1 H), |
| | | 7.24 (dtd, 1 H), 7.07 (t, J = 8.9 Hz, 1 H), 5.44-5.67 (m, 1 H), 4.59- |
| | | 4.71 (m, 2 H), 4.46-4.56 (m, 1 H), 4.28 (br d, J = 13.2 Hz, 1 H), |
| | | 3.82-4.08 (m, 3 H), 3.35-3.52 (m, 3 H), 2.54-2.77 (m, 2 H), |
| | | 2.31-2.49 (m, 3 H), 2.09-2.24 (m, 2 H), 1.70-1.89 (m, 3 H), |
| | | 1.52-1.67 (m, 2 H), 1.02 (t, J = 7.5 Hz, 3 H). |
| 38 | 717.0 | ¹H NMR (400 MHz, METHANOL-d₄) δ ppm 8.01 (t, J = 1.8 Hz, 1 |
| | | H), 7.24 (dd, J = 8.4, 5.2 Hz, 1 H), 7.07 (dd, J-9.3, 8.5 Hz, 1 H), |
| | | 5.43-5.72 (m, 1 H), 4.80-4.91 (m, 1 H), 4.64-4.77 (m, 2 H), |
| | | 4.48-4.62 (m, 1 H), 4.20-4.28 (m, 1 H), 3.83-4.10 (m, 4 H), |
| | | 3.66-3.78 (m, 1 H), 3.35-3.52 (m, 3 H), 3.03 (d, J = 13.8 Hz, 1 |
| | | H), 2.44-2.82 (m, 3 H), 2.12-2.39 (m, 5 H), 1.98-2.07 (m, 1 H), |
| | | 1.78-1.98 (m, 3 H). |
| 39 | 643.0 | ¹H NMR (400 MHz, METHANOL-d₄) δ ppm 8.20 (dd, J = 3.7, 1.6 |
| | | Hz, 1 H), 7.24 (dt, J-8.4, 5.2 Hz, 1 H), 7.07 (t, J = 8.9 Hz, 1 H), |
| | | 5.43-5.67 (m, 1 H), 4.57-4.74 (m, 2 H), 4.43 (br t, J = 12.3 Hz, 1 |
| | | H), 4.20 (br d, J = 13.6 Hz, 1 H), 3.82-4.06 (m, 3 H), 3.35-3.61 |
| | | (m, 3 H), 2.55-2.77 (m, 2 H), 2.32-2.50 (m, 3 H), 2.09-2.23 |
| | | (m, 2 H), 1.74-1.90 (m, 3 H), 1.30 (d, J = 3.6 Hz, 3 H). |
| 40 | 667.0 | ¹H NMR (400 MHz, METHANOL-d₄) δ ppm 7.97 (d, J = 1.7 Hz, 1 |
| | | H), 7.25 (t, J = 6.4 Hz, 1 H), 7.07 (dd, J = 9.4, 8,6 Hz, 1 H), 5.46- |
| | | 5.70 (m, 1 H), 4.55-4.77 (m, 3 H), 3.78-4.21 (m, 7 H), 3.45- |
| | | 3.53 (m, 1 H), 2.84-3.00 (m, 3 H), 2.55-2.80 (m, 2 H), 2.32- |
| | | 2.49 (m, 3 H), 2.11-2.25 (m, 1 H), 2.00-2.08 (m, 2 H), 1.93 (br |
| | | dd, J = 11.5, 5.9 Hz, 2 H). |
| 41 | 643.0 | ¹H NMR (400 MHz, METHANOL-d₄) δ ppm 8.21 (dd, J = 5.5, 1.6 |
| | | Hz, 1 H), 7.24 (td, J = 8.3, 5.2 Hz, 1 H), 7.07 (ddd, J = 9.5, 8.4, 1.1 |
| | | Hz, 1 H), 5.44-5.72 (m, 1 H), 4.58-4.75 (m, 2 H), 4.40-4.49 |
| | | (m, 1 H), 4.21 (br d, J = 13.4 Hz, 1 H), 3.83-4.09 (m, 3 H), 3.35- |
| | | 3.62 (m, 3 H), 2.55-2.79 (m, 2 H), 2.31-2.50 (m, 3 H), 2.09- |
| | | 2.24 (m, 2 H), 1.74-1.90 (m, 3 H), 1.30 (d, J = 3.3 Hz, 3 H). |
| 42 | 655.0 | ¹H NMR (400 MHz, METHANOL-d₄) δ ppm 7.96 (d, J = 1.7 Hz, 1 |
| | | H), 7.25 (ddd, J = 8.4, 5.2, 1.0 Hz, 1 H), 7.07 (dd, J = 9.4, 8.4 Hz, 1 |
| | | H), 5.43-5.71 (m, 1 H), 4.61-4.75 (m, 2 H), 4.43-4.54 (m, 4 H), |
| | | 4.16-4.32 (m, 2 H), 3.83-4.09 (m, 5 H), 3.45-3.55 (m, 1 H), |
| | | 2.54-2.80 (m, 2 H), 2.32-2.51 (m, 3 H), 2.01-2.24 (m, 3 H), |
| | | 1.83 (quin, J = 5.6 Hz, 2 H). |
| 43 | 657.0 | ¹H NMR (400 MHz, METHANOL-d₄) δ ppm 8.09 (dd, J = 8.0, 1.6 |
| | | Hz, 1 H), 7.25 (ddd, J = 8.3, 5.1, 3.1 Hz, 1 H), 7.07 (t, J = 8.9 Hz, 1 |
| | | H), 5.46-5.68 (m, 1 H), 4.59-4.75 (m, 2 H), 3.82-4.07 (m, 6 H), |
| | | 3.69 (dd, J = 13.1, 7.8 Hz, 1 H), 3.44-3.60 (m, 2 H), 3.35-3.39 |
| | | (m, 1 H), 2.55-2.77 (m, 2 H), 2.31-2.50 (m, 3 H), 2.09-2.23 |
| | | (m, 1 H), 1.83-1.96 (m, 2 H), 1.71-1.82 (m, 1 H), 1.52-1.61 |
| | | (m, 1 H), 1.03 (d, J = 1.7 Hz, 3 H). |
| 44 | 673.0 | ¹H NMR (400 MHz, METHANOL-d₄) δ ppm 8.47 (d, J = 5.8 Hz, 1 |
| | | H), 7.23 (dd, J = 8.5, 5.1 Hz, 1 H), 7.06 (t, J = 8.6 Hz, 1 H), 5.29- |
| | | 5.61 (m, 1 H), 4.37-4.59 (m, 4 H), 3.97-4.11 (m, 2 H), 3.69- |
| | | 3.95 (m, 4 H), 3.55-3.67 (m, 3 H), 3.22-3.31 (m, 1 H), 2.38- |
| | | 2.60 (m, 2 H), 2.27-2.36 (m, 1 H), 2.16-2.26 (m, 2 H), 1.98- |
| | | 2.11 (m, 1 H), 1.57-1.67 (m, 2 H), 1.06 (td, J = 7.5, 4.1 Hz, 3 H). |
| 45 | 673.0 | ¹H NMR (400 MHz, METHANOL-d₄) δ ppm 8.43 (dd, J = 7.7, 1.5 |
| | | Hz, 1 H), 7.23 (ddd, J-8.3, 5.2, 3.0 Hz, 1 H), 7.05 (dd, J = 9.3, 8.5 |
| | | Hz, 1 H), 5.27-5.50 (m, 1 H), 4.33-4.51 (m, 4 H), 3,97-4.09 |
| | | (m, 2 H), 3.73-3.92 (m, 3 H), 3.40-3.64 (m, 4 H), 3.15-3.31 |
| | | (m, 1 H), 2.19-2.57 (m, 3 H), 2.09-2.17 (m, 2 H), 1.94-2.05 |
| | | (m, 1 H), 1.56-1.67 (m, 2 H), 1.06 (td, J = 7.5, 4.1 Hz, 3 H). |
| 46 | 694.8 | ¹H NMR (400 MHz, METHANOL-d₄) δ ppm 8.37 (d, J = 1.7 Hz, 1 |
| | | H), 7.25 (dd, J-8.4, 5.0 Hz, 1 H), 7.07 (dd, J-9.4, 8.6 Hz, 1 H), |
| | | 5.92 (t, J = 54.9 Hz, 1 H), 5.45-5,68 (m, 1 H), 4.73-4.77 (m, 1 |
| | | H), 4.64 (d, J = 12.8 Hz, 1 H), 4.41-4.51 (m, 2 H), 4.25 (d, J = 15.0 |
| | | Hz, 1 H), 3.86-4.13 (m, 7 H), 3.77 (d, J = 13.2 Hz, 1 H), 3.44- |
| | | 3.53 (m, 1 H), 2.55-2.75 (m, 2 H), 2.30-2.49 (m, 3 H), 2.15 (br |
| | | dd, J = 6.6, 3.0 Hz, 1 H). |
| 47 | 643.2 | ¹H NMR (400 MHz, METHANOL-d₄) δ ppm 8.32 (d, J = 1.5 Hz, 1 |
| | | H), 7.24 (dd, J = 8.4, 5.0 Hz, 1 H), 7.07 (dd, J = 9.4, 8.4 Hz, 1 H), |
| | | 5.24-5.76 (m, 1 H), 4.73 (s, 2 H), 4.57 (d, J = 12.8 Hz, 1 H), 4.18- |
| | | 4.48 (m, 3 H), 3.89 (br d, J = 11.5 Hz, 4 H), 3.35-3.54 (m, 2 H), |
| | | 2.27-2.69 (m, 7 H), 2.07-2.24 (m, 3 H), 1.79-2.00 (m, 1 H), |
| | | 1.68 (s, 3 H) |
| 48 | 643.2 | ¹H NMR (400 MHz, METHANOL-d₄) δ ppm 8.08-8.32 (m, 1 H), |
| | | 7.25 (dd, J = 8.5, 5.1 Hz, 1 H), 7.07 (dd, J = 9.4, 8.6 Hz, 1 H), 5.40- |
| | | 5.67 (m, 2 H), 4.04-4.28 (m, 2 H), 3.84-4.04 (m, 4 H), 3.70- |
| | | 3.84 (m, 3 H), 3.50 (dt, J = 3.2, 1.7 Hz, 2 H), 3.41 (s, 4 H), 2.52- |
| | | 2.81 (m, 2 H), 2.32-2.45 (m, 3 H), 2.17 (s, 5 H), 1.97-2.10 (m, 1 |
| | | H) |
| 49 | 629.2 | ¹H NMR (400 MHz, METHANOL-d₄) δ ppm 8.16-8.39 (m, 1 H), |
| | | 7.18-7.30 (m, 1 H), 7.24 (ddd, J = 8.2, 5.3, 2.6 Hz, 1 H), 7.07 (dd, |
| | | J-9.4, 8.4 Hz, 1 H), 5.20-5.82 (m, 1 H), 4.83 (br d, J = 5.2 Hz, 3 |
| | | H), 4.63-4.74 (m, 1 H), 4.59 (d, J = 12.8 Hz, 1 H), 4.10-4.28 (m, |
| | | 2 H), 3.98-4.10 (m, 1 H), 3.86-3.97 (m, 4 H), 3.80-3.88 (m, 1 |
| | | H), 3.48 (dt, J = 9.7, 5.7 Hz, 1 H), 2.57-2.79 (m, 2 H), 2.37 (br d, |
| | | J = 10.2 Hz, 3 H), 2.19 (br d, J = 6.9 Hz, 3 H), 2.05 (br s, 1 H) |
| 50 | 638.2 | ¹H NMR (400 MHz, METHANOL-d₄) δ ppm 8.07-8.39 (m, 1 H), |
| | | 7.25 (td, J = 8.2, 5.0 Hz, 1 H), 7.07 (dd, J = 9.4, 8.6 Hz, 1 H), 5.44- |
| | | 5.65 (m, 1 H), 4.82 (br s, 3 H), 4.56-4.72 (m, 2 H), 4.16-4.37 |
| | | (m, 2 H), 3.79-4.08 (m, 3 H), 3.49 (br dd, J = 9.7, 5.7 Hz, 1 H), |
| | | 3.11-3.24 (m, 2 H), 2.55-2.86 (m, 2 H), 2.27-2.49 (m, 5 H), |
| | | 1.97-2.20 (m, 3 H) |
| 51 | 643.2 | ¹H NMR (400 MHz, METHANOL-d₄) δ ppm 8.08 (d, J = 1.7 Hz, 1 |
| | | H), 7.26 (ddd, J = 8.4, 5.0, 1.5 Hz, 1 H), 7.02-7.16 (m, 1 H), 5.43- |
| | | 5.72 (m, 1 H), 4.58-4.74 (m, 2 H), 4.33 (t, J = 5.9 Hz, 2 H), 4.06- |
| | | 4.18 (m, 4 H), 3.91-3.98 (m, 2 H), 3.73-3.86 (m, 5 H), 3.45- |
| | | 3.60 (m, 1 H), 2.60-2.72 (m, 1 H), 2.31-2.41 (m, 1 H), 2.30- |
| | | 2.43 (m, 1 H), 2.03-2.16 (m, 2 H), 1.85-1.94 (m, 3 H), 1.67- |
| | | 1.78 (m, 2 H) |
| 52 | 643.2 | ¹H NMR (400 MHz, METHANOL-d₄) δ ppm 8.18-8.31 (m, 1 H), |
| | | 7.19-7.37 (m, 1 H), 6.93-7.11 (m, 1 H), 5.50-5.70 (m, 2 H), |
| | | 4.67-4.73 (m, 1 H), 4.56-4.64 (m, 1 H), 4.12-4.51 (m, 2 H), |
| | | 3.73-4.07 (m, 3 H), 3.50 (br dd, J = 3.2, 1.6 Hz, 2 H), 2.63 (br s, 2 |
| | | H), 2.28-2.44 (m, 3 H), 2.05-2.22 (m, 3 H), 1.86-2.00 (m, 1 H), |
| | | 1.55-1.73 (m, 3 H), 1.34-1.46 (m, 2 H), 0.82-1.04 (m, 2 H) |
| 53 | 624.2 | ¹H NMR (400 MHz, METHANOL-d₄) δ ppm 8.19-8.36 (s, 1 H), |
| | | 7.24 (dd, J = 8.4, 5.0 Hz, 2 H), 6.99-7.14 (s, 1 H), 5.16-5.31 (m, |
| | | 2 H), 4.00-49 (m, 6 H), 3.65-3.92 (m, 2 H), 2.28-2.53 (m, 8 H), |
| | | 2.17 (s, 1 H), 1.98 (d, J = 19.0 Hz, 1 H), 1.24-1.56 (m, 2 H) |
| 54 | 638.2 | ¹H NMR (400 MHz, METHANOL-d₄) δ ppm 7.89-8.08 (m, 1 H), |
| | | 7.25 (dd, J = 8.4, 5.0 Hz, 1 H), 7.07 (dd, J = 9.3, 8.5 Hz, 1 H), 5.42- |
| | | 5.77 (m, 1 H), 4.61-4.69 (m, 1 H), 4.33 (d, J = 5.6 Hz, 1 H), 3.73- |
| | | 4.19 (m, 7 H), 3.41-3.63 (m, 2 H), 3,17-3.30 (m, 1 H), 2.55- |
| | | 2.84 (m, 3 H), 2.31-2.53 (m, 3 H), 2.02-2.27 (m, 4 H), 1.75- |
| | | 1.96 (m, 1 H) |
| 65 | 629.2 | ¹H NMR (400 MHz, METHANOL-d₄) δ ppm 8.21 (dd, J = 3.8, 1,5 |
| | | Hz, 1 H), 6.94-7.36 (m, 2 H), 5.72 (s, 3 H), 4.52-4.76 (m, 3 H), |
| | | 4.45 (br d, J = 5.0 Hz, 2 H), 3.75-4.25 (m, 5 H), 3.38-3.67 (m, 1 |
| | | H), 2.55-2.67 (m, 1 H), 2.27-2.49 (m, 3 H), 1.79-2.25 (m, 4 H), |
| | | 1.49 (d, J = 6.5 Hz, 3 H), 1.32 (s, 1 H). |
| 66 | 613.2 | ¹H NMR (400 MHz, METHANOL-d₄) δ ppm 8.25 (t, J = 2.0 Hz, 1 |
| | | H), 7.25 (ddd, J = 8.4, 5.0, 3.6 Hz, 1 H), 7.08 (dd, J = 9.3, 8.5 Hz, 1 |
| | | H), 5.36-5.84 (m, 1 H), 4.55-4.80 (m, 4 H), 4.00-4.33 (m, 2 H), |
| | | 3.77-3.99 (m, 2 H), 3.49 (br dd, J = 9.6, 5.6 Hz, 2 H), 2.52-2.80 |
| | | (m, 2 H), 1.79-2.44 (m, 8 H), 1.48 (d, J = 6.3 Hz, 3 H). |
| 67 | 590.0 | ¹H NMR (400 MHz, METHANOL-d₄) δ ppm 8.82 (br d, J = 8.9 |
| | | Hz, 1 H), 7.28 (dt, J = 8.4, 4.8 Hz, 1 H), 7.09 (t, J = 8.6 Hz, 1 H), |
| | | 4.60-4.82 (m, 2 H), 4.53 (dd, J-10.6, 6.6 Hz, 1 H), 4.42 (dd, |
| | | J = 10.6, 6.0 Hz, 1 H), 4.18-4.31 (m, 1 H), 3.91-4.13 (m, 3 H), |
| | | 3.66-3.79 (m, 2 H), 1.37 (s, 6 H), 1.32 (s, 3 H). |

### 2-Amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile (Example 55)

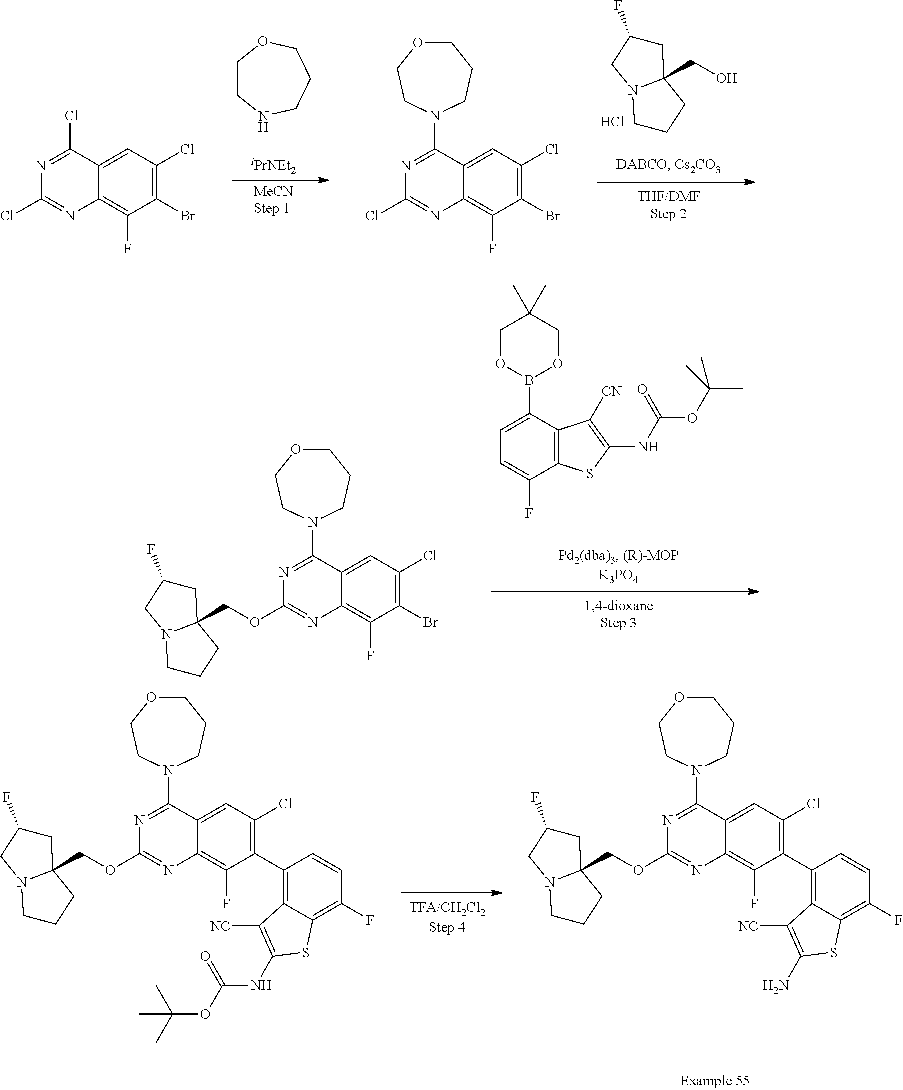

Step 1: 4-(7-Bromo-2,6-dichloro-8-fluoroquinazolin-4-yl)-1,4-oxazepane. To a mixture of 7-bromo-2,4,6-trichloro-8-fluoroquinazoline (2.03 g, 6.13 mmol, WuXi App Tec Co. Ltd.) in acetonitrile (15 mL) was added DIPEA (0.83 g, 1.1 mL, 6.44 mmol, Sigma-Aldrich Corporation) and [1,4]oxazepane (0.63 g, 6.25 mmol, Oakwood Products, Inc.). The reaction was stirred at rt overnight. Solvent was removed under reduced pressure. The crude product was purified by column chromatography on silica gel, eluting with a gradient of 0-50% EtOAc in heptane to yield 4-(7-bromo-2,6-dichloro-8-10 fluoroquinazolin-4-yl)-1,4-oxazepane (0.53 g, 1.34 mmol, 22% yield). m/z (ESI): 394.0 (M+H)⁺.
Step 2: 4-(7-Bromo-6-chloro-8-fluoro-2-(((2R,7aS)-2-fluorotetrahydro-1H-pyrrolizin-7a(5H)-yl)methoxy)quinazolin-4-yl)-1,4-oxazepane. A 20-mL vial was charged with 1,4-diazabicyclo[2.2.2]octane (15 mg, 0.13 mmol, Sigma-Aldrich Corporation), cesium carbonate (2.61 g, 8.02 mmol, Sigma-Aldrich Corporation), ((2R,7aS)-2-fluorotetrahydro-1H-pyrrolizin-7a(5H)-yl) methanol hydrochloride (0.79 g, 4.01 mmol, AChemBlock), 4-(7-bromo-2,6-dichloro-8-fluoroquinazolin-4-yl)-1,4-oxazepane (0.53 g, 1.34 mmol), N,N-dimethylformamide (2.2 mL) and tetrahydrofuran (4.5 mL). The reaction was stirred at 40° C. overnight. The crude mixture was purified by column chromatography on silica gel, eluting with a gradient of 0-50% 3:1 EtOAc/EtOH in heptane with 2% triethylamine additive to yield 4-(7-bromo-6-chloro-8-fluoro-2-(((2R,7aS)-2-fluorotetrahydro-1H-pyrrolizin-7a(5H)-yl)methoxy)quinazolin-4-yl)-1,4-oxazepane (0.27 g, 0.52 mmol, 39% yield). m/z (ESI): 517.0 (M+H)⁺.
Step 3: tert-Butyl (4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-3-cyano-7-fluorobenzo[b]thiophen-2-yl) carbamate. An 8-mL vial was charged with potassium phosphate (0.54 g, 2.55 mmol, Combi-Blocks Inc.), (R)-(+)-2-(diphenylphosphino)-2′-methoxy-1, 1′-binaphthyl (48 mg, 0.10 mmol, Strem Chemicals, Inc.), bis[tris(dibenzylideneacetone)palladium (0)] (47 mg, 0.05 mmol, Sigma-Aldrich Corporation), and tert-butyl (3-cyano-4-(5,5-dimethyl-1,3,2-dioxaborinan-2-yl)-7-fluorobenzo[b]thiophen-2-yl)carbamate (0.62 g, 1.53 mmol, PharmaBlock), 4-(7-bromo-6-chloro-8-fluoro-2-(((2R,7aS)-2-fluorotetrahydro-1H-pyrrolizin-7a(5H)-yl)methoxy)quinazolin-4-yl)-1,4-oxazepane (0.26 g, 0.51 mmol), and 1,4-dioxane (5.0 mL). The reaction mixture was stirred at 80° C. for 1 h. The crude mixture was purified by column chromatography on silica gel, eluting with a gradient of 0-50% 3:1 EtOAc/EtOH in heptane with 2% triethylamine additive to yield tert-butyl (4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-3-cyano-7-fluorobenzo[b]thiophen-2-yl)carbamate (0.21 g, 0.29 mmol, 56% yield). m/z (ESI): 729.7 (M+H)⁺.
Step 4: 2-Amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile. tert-Butyl (4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-3-cyano-7-fluorobenzo[b]thiophen-2-yl)carbamate (0.21 g, 0.29 mmol) was stirred in trifluoroacetic acid (0.7 mL) and dichloromethane (0.7 mL) at rt for 1 h. Volatiles were removed under reduced pressure. The crude product was purified by reverse phase HPLC to yield 2-amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile as bis(2,2,2-trifluoroacetate) as light-yellow solid (89 mg, 0.10 mmol, 36% yield). m/z (ESI): 629.8 (M+H)⁺. ¹H NMR (400 MHZ, METHANOL-d₄) δ ppm 8.10-8.14 (m, 1H), 7.21-7.28 (m, 1H), 7.03-7.11 (m, 1H), 5.46-5.66 (m, 1H), 4.57-4.76 (m, 2H), 4.17-4.30 (m, 4H), 3.83-4.10 (m, 7H), 3.42-3.54 (m, 1H), 2.59 (br s, 2H), 2.37 (br d, J=7.9 Hz, 3H), 2.06-2.27 (m, 3H).

TABLE 5
Additional Examples 56 to 60, 68 to 69. Prepared in an Analogous Manner to Example 55.

| Ex | | | Salt | | Method |
|---|---|---|---|---|---|
| # | Structure | Name | Form | Reagent | Change |
| 56 | 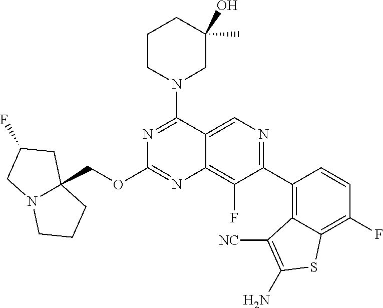 [SMILES: C[C@@]1(O)CCCN(c2nc(OC[C@@]34CCCN3C[C@H](F)C4)nc3c(F)c(-c4ccc(F)c5sc(N)c(C#N)c45)ncc23)C1] | 2-Amino-7- fluoro-4-(8- fluoro-2- (((2R,7aS)-2- fluorotetrahydro- 1H-pyrrolizin- 7a(5H)- yl)methoxy)-4- ((R)-3-hydroxy- 3- methylpiperidin- 1-yl)pyrido[4,3- d]pyrimidin-7- yl)benzo[b] thiophene-3- carbonitrile | Bis(2, 2,2- tri- fluoro- acetate) | Step 1: (3R)- 3- methylpiperidin- 3-ol hydrochloride (CAS#: 2305080-34-4, PharmaBlock) and 2,4,7- trichloro-8- fluoro- pyrido[4,3- d]pyrimidine (CAS#: 2454396-80-4, LabNetwork) | Step 1 and 2 were carried out in one pot with DIPEA/ MeCN instead of DABCO/ Cs₂CO₃/ DMF/THF. |
| 57 | 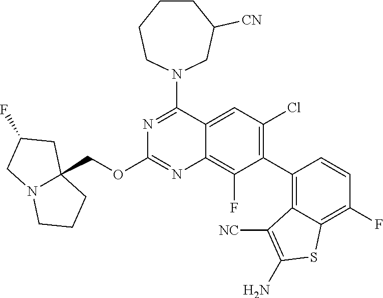 [SMILES: N#Cc1c(N)sc2c(F)ccc(-c3c(Cl)cc4c(N5CCCCC(C#N)C5)nc(OC[C@@]56CCCN5C[C@H](F)C6)nc4c3F)c12] | 1-(7-(2-Amino-3- cyano-7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)azepane-3- carbonitrile | Bis(2, 2,2- tri- fluoro- acetate) | Step 1: azepane-3- carbonitrile hydrochloride (CAS#: 2007917-48-6, Pharmablock, Inc.) | |
| 58 | 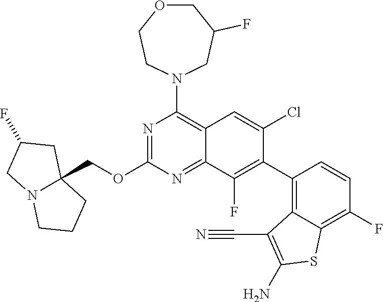 [SMILES: N#Cc1c(N)sc2c(F)ccc(-c3c(Cl)cc4c(N5CCOCC(F)C5)nc(OC[C@@]56CCCN5C[C@H](F)C6)nc4c3F)c12] | 2-Amino-4-(6- chloro-8-fluoro- 4-(6-fluoro-1,4- oxazepan-4-yl)- 2-(((2R,7aS)-2- fluorotetrahydro- 1H-pyrrolizin- 7a(5H)- yl)methoxy) quinazolin-7-yl)-7- fluorobenzo[b] thiophene-3- carbonitrile Isomer 1 | Bis (2,2,2- tri- fluoro- aceate) | Step 1: 6- fluoro-1,4- oxazepane hydrochloride (CAS#: 218594-82-2, Enamine) | Chiral separation after Step 2. Details included below. |
| 59 | 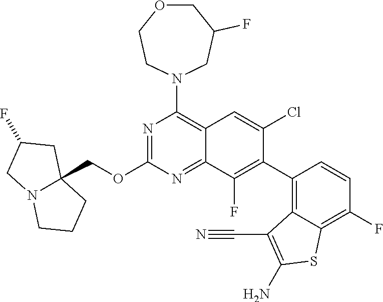 [SMILES: N#Cc1c(N)sc2c(F)ccc(-c3c(Cl)cc4c(N5CCOCC(F)C5)nc(OC[C@@]56CCCN5C[C@H](F)C6)nc4c3F)c12] | 2-Amino-4-(6- chloro-8-fluoro- 4-(6-fluoro-1,4- oxazepan-4-yl)- 2-(((2R,7aS)-2- fluorotetrahydro- 1H-pyrrolizin- 7a(5H)- yl)methoxy) quinazolin-7-yl)-7- fluorobenzo[b] thiophene-3- carbonitrile Isomer 2 | Bis (2,2.2- tri- fluoro- aceate) | Step 1: 6- fluoro-1,4- oxazepane hydrochloride (CAS#: 218594-82-2, Enamine) | Chiral separation after Step 2. Details included below. |
| 60 | 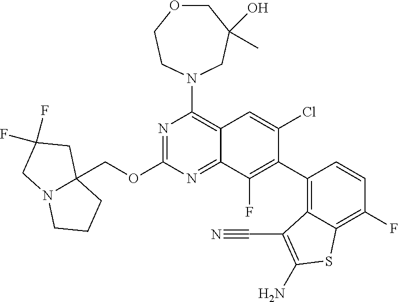 [SMILES: CC1(O)COCCN(c2nc(OCC34CCCN3CC(F)(F)C4)nc3c(F)c(-c4ccc(F)c5sc(N)c(C#N)c45)c(Cl)cc23)C1] | 2-Amino-4-(6- chloro-2-((2,2- difluorotetrahydro- 1H-pyrrolizin- 7a(5H)- yl)methoxy)-8- fluoro-4-(6- hydroxy-6- methyl-1,4- oxazepan-4- yl)quinazolin-7- yl)-7- fluorobenzo[b] thiophene-3- carbonitrile | Bis (2,2,2- tri- fluoro- aceate) | Step 1: 6- methyl-1,4- oxazepan-6-ol hydrochloride isomer 1 (Intermediate A1) Step 2: (2,2- difluorotetra- hydro-1H- pyrrolizin- 7a(5H)- yl)methanol (CAS#: 1788873-48-2, PharmaBlock Inc.) | |
| 68 | 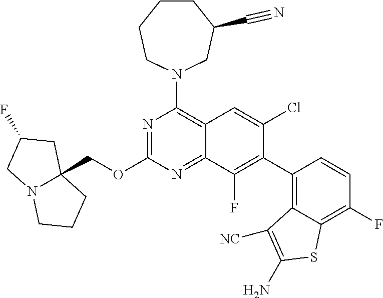 [SMILES: N#Cc1c(N)sc2c(F)ccc(-c3c(Cl)cc4c(N5CCCC[C@@H](C#N)C5)nc(OC[C@@]56CCCN5C[C@H](F)C6)nc4c3F)c12] | (3R)-1-(7-(2- amino-3-cyano- 7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)azepane-3- carbonitrile | 2,2,2- tri- fluoro- acetate | Step 1: azepane-3- carbonitrile hydrochloride (CAS#: 2007917-48-6, Pharmablock, Inc.) | Chiral separation performed after Step 1. Details included below. |
| 69 | 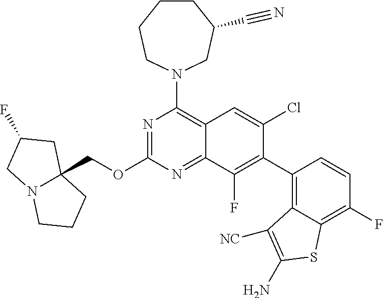 [SMILES: N#Cc1c(N)sc2c(F)ccc(-c3c(Cl)cc4c(N5CCCC[C@H](C#N)C5)nc(OC[C@@]56CCCN5C[C@H](F)C6)nc4c3F)c12] | (3S)-1-(7-(2- amino-3-cyano- 7- fluorobenzo[b] thiophen-4-yl)-6- chloro-8-fluoro- 2-(((2R,7aS)-2- fluorotetrahydro- 1H-pyrrolizin- 7a(5H)- yl)methoxy) quinazolin-4- yl)azepane-3- carbonitrile | 2,2,2- tri- fluoro- acetate | Step 1: azepane-3- carbonitrile hydrochloride (CAS#: 2007917-48-6, PharmaBlock, Inc.) | Chiral separation performed after Step 1. Details included below. |

TABLE 6
Conditions for Chiral SFC Separation.

| Separation | SFC Conditions | Peak to Ex# |
|---|---|---|
| 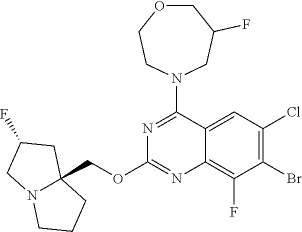 [SMILES: Fc1c(Br)c(Cl)cc2c(N3CCOCC(F)C3)nc(OC[C@@]34CCCN3C[C@H](F)C4)nc12] | Column: ChiralPak AD (2 x 25 cm, 5 μm). Mobile phase: 25% 1:1 ACN. MeOH with 0.2% TEA. Flowrate: 80 mL/min. Yield: 584 mg sample was submitted to generate 114 mg of peak 1 with an ee of 99% and 108 mg of peak 2 with an ee of 99%. | |
| 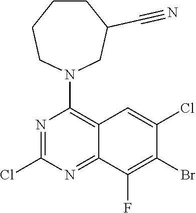 [SMILES: N#CC1CCCCN(c2nc(Cl)nc3c(F)c(Br)c(Cl)cc23)C1] | Column: Chiralcel OJ, 2 x 25 cm 5 μm column Mobile phase: 25% MeOH Flowrate: 70 mL/min. Yield: 465 mg sample was submitted to generate 153 mg of peak 1 with an ee of 99% and 169 mg of peak 2 with an ee of 89%. | Peak 1: Example 68 Peak 2: Example 69 |

TABLE 7
Analytical Data for Examples 56 to 60, 68 to 69.

| | MS | |
|---|---|---|
| Ex. | m/z (ESI): | |
| # | (M + H)⁺ | ¹H NMR |
| 56 | 610.2 | ¹H NMR (400 MHz, METHANOL-d₄) δ ppm 9.40 (s, 1 H), 7.44 |
| | | (dd, J = 8.4, 5.0 Hz, 1 H), 7.11 (dd, J = 9.3, 8.5 Hz, 1 H), 5.52-5.72 |
| | | (m, 1 H), 4.82 (br d, J = 9.6 Hz, 3 H), 4.26 (br d, J = 12.8 Hz, 1 H), |
| | | 3.86-4.02 (m, 3 H), 3.38-3.59 (m, 3 H), 3.08-3.18 (m, 2 H), |
| | | 2.60-2.80 (m, 2 H), 2.35-2.56 (m, 3 H), 2.17-2.30 (m, 1 H), |
| | | 1.91-2.10 (m, 5 H). |
| 57 | 652.0 | ¹H NMR (400 MHz, METHANOL-d₄) δ ppm 8.12-8.19 (m, 1 H), |
| | | 7.24 (s, 1 H), 7.07 (dd, J = 9.3, 8.5 Hz, 1 H), 5.43-5.69 (m, 1 H), |
| | | 4.65-4.77 (m, 2 H), 4.38-4.48 (m, 1 H), 4.16-4.34 (m, 2 H), |
| | | 3.81-4.13 (m, 4 H), 3.38-3.56 (m, 2 H), 2.54-2.87 (m, 2 H), |
| | | 2.30-2.53 (m, 3 H), 1.96-2.29 (m, 5 H), 1.79-1.96 (m, 1 H), |
| | | 1.47-1.67 (m, 1 H). |
| 58 | 647.0 | ¹H NMR (400 MHz, METHANOL-d₄) δ ppm 8.19 (d, J = 6.1 Hz, 1 |
| | | H), 7.22-7.27 (m, 1 H), 7.07 (t, J = 8.9 Hz, 1 H), 5,45-5.68 (m, 1 |
| | | H), 5.04-5.26 (m, 1 H), 4.69 (s, 1 H), 4.61 (d, J = 12.5 Hz, 1 H), |
| | | 4.32-4.56 (m, 2 H), 4.04-4.29 (m, 4 H), 3.85-4.01 (m, 5 H), |
| | | 3.44-3.53 (m, 1 H), 2.54-2.77 (m, 2 H), 2.31-2.49 (m, 3 H), |
| | | 2.09-2.24 (m, 1 H). |
| 59 | 647.2 | ¹H NMR (400 MHz, METHANOL-d₄) δ ppm 8.19 (d, J = 6.7 Hz, 1 |
| | | H), 7.25 (ddd, J = 8.4, 5.1, 2.0 Hz, 1 H), 7.07 (dd, J = 9.2, 8.6 Hz, 1 |
| | | H), 5.46-5.66 (m, 1 H), 5,07-5.28 (m, 1 H), 4.58-4.74 (m, 2 H), |
| | | 3.83-4.53 (m, 11 H), 3.44-3.54 (m, 1 H), 2.54-2.81 (m, 2 H), |
| | | 2.31-2.50 (m, 3 H), 2.09-2.24 (m, 1 H). |
| 60 | 677.0 | ¹H NMR (400 MHz, METHANOL-d₄) δ ppm 8.58 (s, 1 H), 7.25 |
| | | (t, J = 6.3 Hz, 1 H), 7.07 (t, J = 9.0 Hz, 1 H), 4.63-4.71 (m, 1 H), |
| | | 4.43-4.60 (m, 2 H), 4.10-4.29 (m, 2 H), 3.84-4.04 (m, 5 H), |
| | | 3.64-3.75 (m, 2 H), 3.35-3.53 (m, 2 H), 2.78-3.03 (m, 2 H), |
| | | 2.47 (dt, J = 13.0, 6.3 Hz, 1 H), 2.21-2.42 (m, 3 H), 1.28-1.32 (m, |
| | | 3 H). |
| 68 | 652.0 | ¹H NMR (400 MHz, METHANOL-d₄) δ ppm 8.21 (d, J = 1.46 Hz, |
| | | 1 H), 7.18-7.29 (m, 1 H), 7.03-7.14 (m, 1 H), 5.93 (s, 2 H), 5.42- |
| | | 5.64 (m, 1 H), 4.60-4.80 (m, 3 H), 3.79-4.09 (m, 3 H), 3.68- |
| | | 3.79 (m, 2 H), 3.42-3.55 (m, 1 H), 2.53-2.80 (m, 2 H), 2.28- |
| | | 2.53 (m, 5 H), 2.08-2.26 (m, 1 H), 1.65-1.88 (m, 4 H). |
| 69 | 652.0 | ¹H NMR (400 MHz, METHANOL-d₄) δ ppm 8.20 (d, J = 1.67 Hz, |
| | | 1 H), 7.20-7.28 (m, 1 H), 7.07 (d, J = 0.84 Hz, 1 H), 5.91 (d, |
| | | J = 15.47 Hz, 2 H), 5.44-5.67 (m, 1 H), 4.56-4.79 (m, 3 H), 3.77- |
| | | 4.13 (m, 3 H), 3.66-3.77 (m, 2 H), 3.41-3.56 (m, 1 H), 2.58 (s, 2 |
| | | H), 2.42 (br t, J = 7.32 Hz, 5 H), 2.05-2.22 (m, 1 H), 1.66-1.90 |
| | | (m, 4 H). |

### 2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile (Example 61)

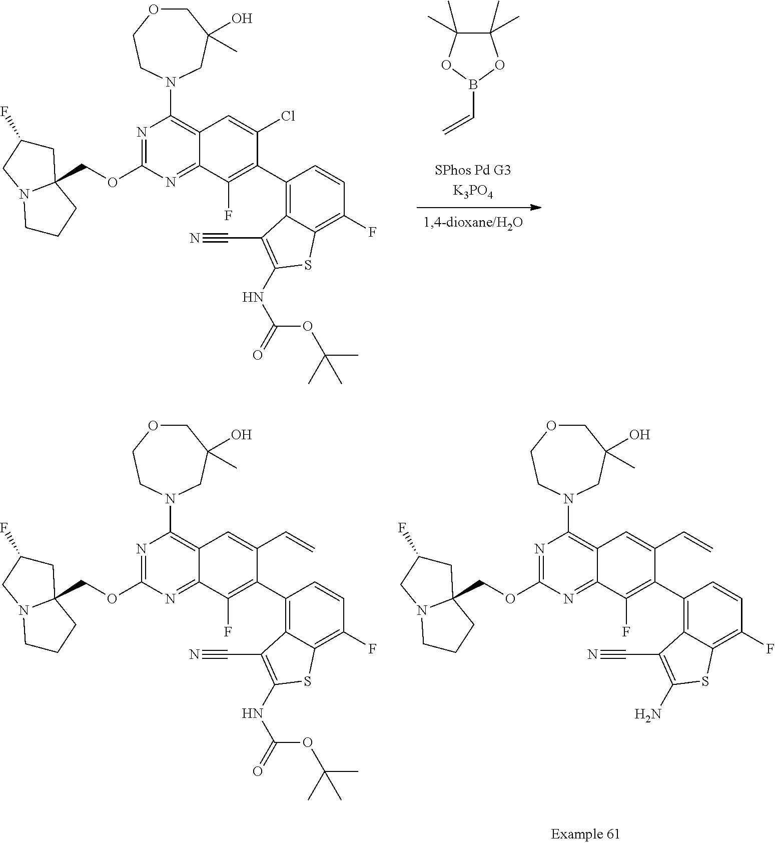

To a 2.0 mL microwave vial was added tert-butyl (4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-3-cyano-7-fluorobenzo[b]thiophen-2-yl)carbamate (39 mg, 0.05 mmol, prepared according to Example 22), vinylboronic acid pinacol ester (40 mg, 0.26 mmol, TCI America), SPhos Pd G3 (18 mg, 0.02 mmol, Sigma-Aldrich Corporation), and potassium phosphate (57 mg, 0.27 mmol, Acros Organics). The vial was purged with nitrogen and suspended in 1,4-dioxane (2.0 mL) and water (0.4 mL), The reaction was then heated in a microwave reactor at 150° C. for 45 min. The reaction mixture was purified by reverse phase HPLC to afford tert-butyl (3-cyano-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophen-2-yl)carbamate (8.0 mg, 10.65 μmol, 21% yield) as orange solid. m/z (ESI): 751.0 (M+H)⁺, and 2-amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile as bis(2,2,2-trifluoroacetate) and as yellow solid (8 mg, 9.1 μmol, 18% yield). m/z (ESI): 651.0 (M+H)⁺. ¹H NMR (400 MHZ, METHANOL-d₄) 8 ppm 8.66 (d, J=3.3 Hz, 1H), 7.18 (ddd, J=8.4, 5.1, 3.4 Hz, 1H), 7.04-7.11 (m, 1H), 6.34-6.43 (m, 1H), 5.85 (dd, J=17.0, 3.4 Hz, 1H), 5.45-5.65 (m, 1H), 5.26 (d, J=11.1 Hz, 1H), 4.58-4.72 (m, 2H), 3.82-4.18 (m, 8H), 3.61-3.81 (m, 2H), 3.45-3.54 (m, 1H), 2.55-2.75 (m, 2H), 2.29-2.54 (m, 4H), 2.08-2.24 (m, 1H), 1.26-1.36 (m, 3H).

### 2-Amino-4-(6-ethyl-8-fluoro-2-(((2R,7aS)-2-fluorotetrahydro-1H-pyrrolizin-7a(H)-yl)methoxy)-4-(6-hydroxy-6-methyl-1,4-oxazepan-4-yl)quinazolin-7-yl)-7-fluorobenzo[b]thiophene-3-carbonitrile (Example 62)

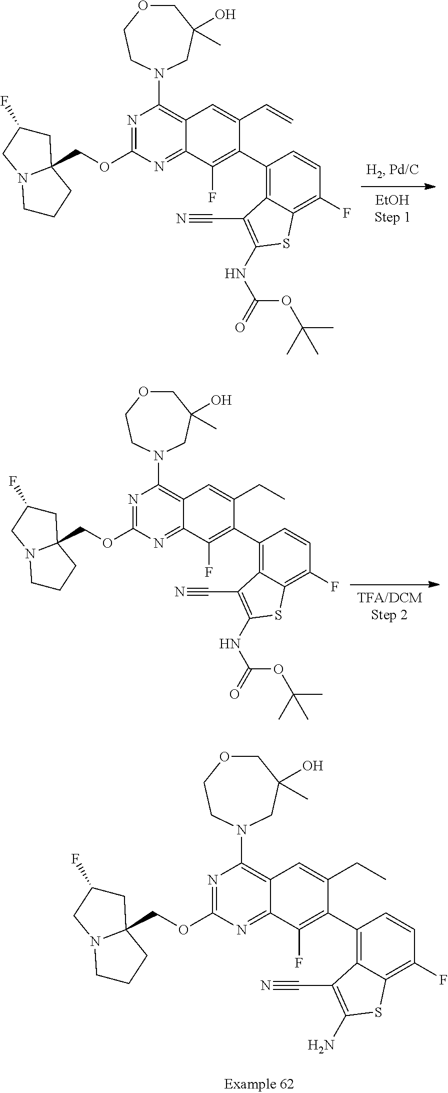

Step 1: tert-Butyl (3-cyano-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophen-2-yl)carbamate. tert-Butyl (3-cyano-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophen-2-yl)carbamate (8 mg, 10.7 μmol, prepared according to Example 61) was dissolved in ethanol (1.0 mL). Palladium on activated carbon (2.3 mg, 2.13 μmol, Sigma-Aldrich Corporation) was added and the mixture was stirred at it under an atmosphere of H₂ (40 psi) for 4 h. The reaction mixture was filtered through celite and washed with MeOH. The filtrate was concentrated and purified by reverse phase HPLC to afford tert-butyl (3-cyano-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophen-2-yl)carbamate as white solid, which was used directly in the next step. m/z (ESI): 752.8 (M+H)⁺.
Step 2: 2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile. The abovementioned tert-butyl (3-cyano-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophen-2-yl)carbamate was dissolved in DCM (0.5 mL) and trifluoroacetic acid (0.5 mL). The reaction mixture was stirred at rt for 1 h. The reaction mixture was concentrated under reduced pressure. The crude product was purified by reverse phase HPLC to afford 2-amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile as bis(2,2,2-trifluoroacetate) and as white solid (2.1 mg, 2.38 μmol, 22% yield). m/z (ESI): 652.9 (M+H)⁺. ¹H NMR (400 MHZ, METHANOL-d₄) δ ppm 8.24 (d, J=12.5 Hz, 1H), 7.21 (ddd, J=8.3, 5.1, 3.3 Hz, ¹H), 7.07 (ddd, J=9.5, 8.3, 1.5 Hz, 1H), 5.43-5.66 (m, 1H), 4.62-4.77 (m, 3H), 4.47-4.60 (m, 2H), 4.09-4.15 (m, 1H), 4.02-4.07 (m, 1H), 3.84-4.01 (m, 5H), 3.65-3.76 (m, 2H), 3.43-3.54 (m, 1H), 2.45-2.66 (m, 4H), 2.31-2.43 (m, 2H), 2.08-2.23 (m, 1H), 1.29 (s, 3H), 1.08-1.15 (m, 3H).

### 2-Amino-7-fluoro-4-(8-fluoro-2-(((2R,7aS)-2-fluorotetrahydro-1H-pyrrolizin-7a (H)-yl)methoxy)-6-hydroxy-4-(6-hydroxy-6-methyl-1,4-oxazepan-4-yl)quinazolin-7-yl)benzo[b]thiophene-3-carbonitrile (Example 63)

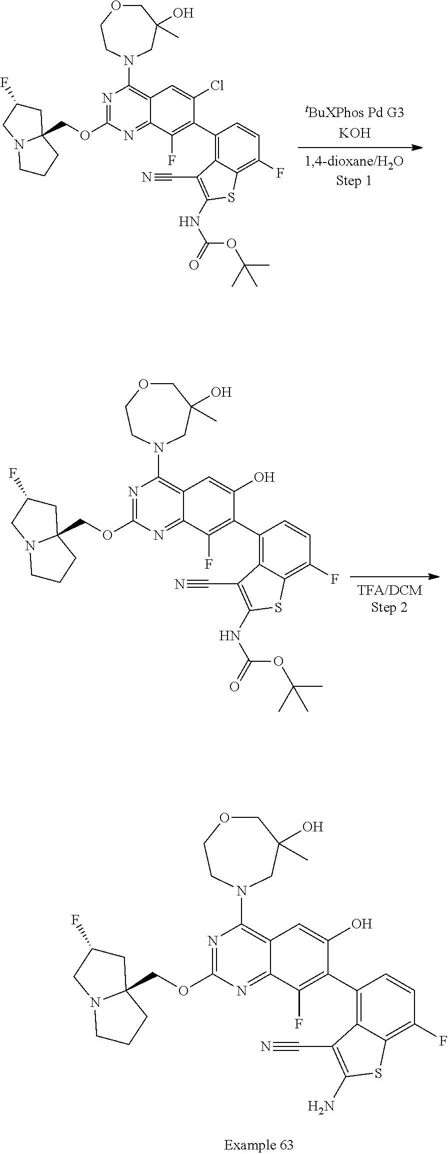 [SMILES: CC1(O)COCCN(c2nc(OC[C@@]34CCCN3C[C@H](F)C4)nc3c(F)c(-c4ccc(F)c5sc(NC(=O)OC(C)(C)C)c(C#N)c45)c(Cl)cc23)C1 ; CC1(O)COCCN(c2nc(OC[C@@]34CCCN3C[C@H](F)C4)nc3c(F)c(-c4ccc(F)c5sc(NC(=O)OC(C)(C)C)c(C#N)c45)c(O)cc23)C1 ; CC1(O)COCCN(c2nc(OC[C@@]34CCCN3C[C@H](F)C4)nc3c(F)c(-c4ccc(F)c5sc(N)c(C#N)c45)c(O)cc23)C1]

Step 1: tert-Butyl (3-cyano-7-fluoro-4-(8-fluoro-2-(((2R,7aS)-2-fluorotetrahydro-1H-pyrrolizin-7a(5H)-yl)methoxy)-6-hydroxy-4-(6-hydroxy-6-methyl-1,4-oxazepan-4-yl)quinazolin-7-yl)benzo[b]thiophen-2-yl)carbamate. The mixture of tert-butyl (4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-3-cyano-7-fluorobenzo[b]thiophen-2-yl)carbamate (61 mg, 0.08 mmol, prepared according to Example 22), potassium hydroxide (23 mg, 0.40 mmol, VWR International, LLC), and tBuXPhos Pd G3 (6.4 mg, 8.03 μmol, Sigma-Aldrich Corporation) in 1,4-dioxane (2.0 mL) and water (0.5 mL) was stirred at 100° C. for 1.S h. The reaction mixture was purified by reverse phase HPLC to afford tert-butyl (3-cyano-7-fluoro-4-(8-fluoro-2-(((2R,7aS)-2-fluorotetrahydro-1H-pyrrolizin-7a(5H)-yl)methoxy)-6-hydroxy-4-(6-hydroxy-6-methyl-1,4-oxazepan-4-yl)quinazolin-7-yl)benzo[b]thiophen-2-yl)carbamate (30 mg, 0.04 mmol, 50% yield) as orange solid. m/z (ESI): 741.0 (M+H)⁺.
Step 2: 2-Amino-7-fluoro-4-(8-fluoro-2-(((2R,7aS)-2-fluorotetrahydro-1-pyrrolizin-7a(5H)-yl)methoxy)-6-hydroxy-4-(6-hydroxy-6-methyl-1,4-oxazepan-4-yl)quinazolin-7-yl)benzo[b]thiophene-3-carbonitrile. tert-Butyl (3-cyano-7-fluoro-4-(8-fluoro-2-(((2R,7aS)-2-fluorotetrahydro-1H-pyrrolizin-7a(5H)-yl)methoxy)-6-hydroxy-4-(6-hydroxy-6-methyl-1,4-oxazepan-4-yl)quinazolin-7-yl)benzo[b]thiophen-2-yl)carbamate (30 mg, 0.04 mmol) was dissolved in DCM (1.0 mL) and trifluoroacetic acid (0.5 mL). The reaction mixture was stirred at rt for 1 h. The reaction mixture was concentrated under reduced pressure. The crude product was purified by reverse phase HPLC to afford 2-amino-7-fluoro-4-(8-fluoro-2-(((2R,7aS)-2-fluorotetrahydro-1H-pyrrolizin-7a(5H)-yl)methoxy)-6-hydroxy-4-(6-hydroxy-6-methyl-1,4-oxazepan-4-yl)quinazolin-7-yl)benzo[b]thiophene-3-carbonitrile as bis(2,2,2-trifluoroacetate) as yellow solid (20 mg, 0.02 mmol, 57% yield). m/z (ESI): 641.0 (M+H)⁺. ¹H NMR (400 MHZ, METHANOL-d₄) δ ppm 7.57 (s, 1H), 7.25 (ddd, J=8.4, 5.0, 4.2 Hz, 1H), 7.04 (ddd, J=9.5, 8.5, 2.1 Hz, 1H), 5.45-5.69 (m, 1H), 4.56-4.73 (m, 2H), 4.38-4.49 (m, 2H), 3.84-4.16 (m, 7H), 3.63-3.75 (m, 2H), 3.43-3.53 (m, 1H), 2.54-2.71 (m, 2H), 2.30-2.52 (m, 3H), 2.08-2.22 (m, 1H), 1.28 (s, 3H).

### 2-Amino-4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-7-fluorobenzo[b]thiophene-3-carbonitrile (Example 64)

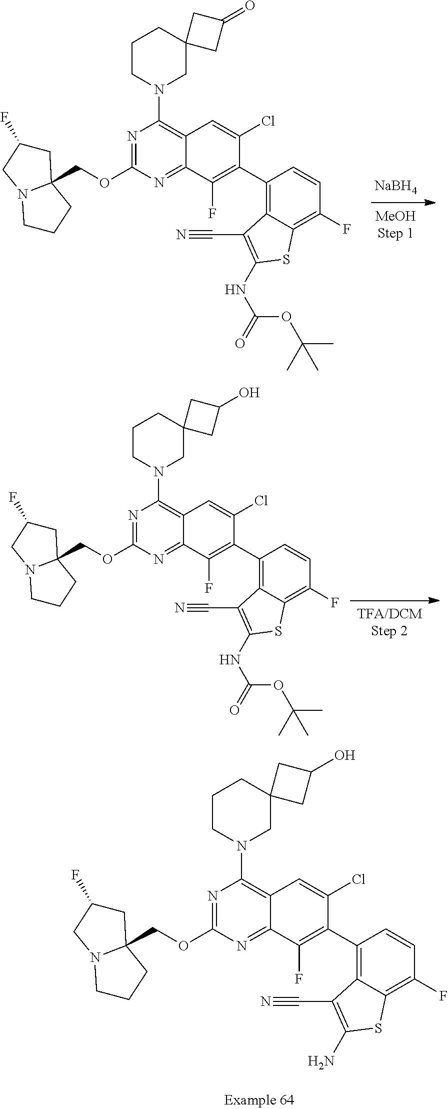

Step 1: tert-Butyl (4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-3-cyano-7-fluorobenzo[b]thiophen-2-yl)carbamate. To a stirred solution of tert-butyl (4-(6-chloro-8-fluoro-2-(((2R,7aS)-2-fluorotetrahydro-1H-pyrrolizin-7a(5H)-yl)methoxy)-4-(2-oxo-6-azaspiro[3.5]nonan-6-yl)quinazolin-7-yl)-3-cyano-7-fluorobenzo[b]thiophen-2-yl)carbamate (44 mg, 0.06 mmol, synthesized according to Example 40) in methanol (1.0 mL) at 0° C. was added sodium borohydride (3.2 mg, 0.09 mmol, Sigma-Aldrich Corporation). The reaction mixture was stirred at 0° C. for 30 min. The reaction was quenched with water. The crude mixture was purified by reverse phase HPLC to afford tert-butyl (4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-3-cyano-7-fluorobenzo[b]thiophen-2-yl)carbamate (20 mg, 0.03 mmol, 46% yield) as yellow solid. m/z (ESI): 769.2 (M+H)⁺.
Step 2: 2-Amino-4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-7-fluorobenzo[b]thiophene-3-carbonitrile. tert-Butyl (4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-3-cyano-7-fluorobenzo[b]thiophen-2-yl)carbamate (20 mg, 0.03 mmol) was dissolved in DCM (0.5 mL) and trifluoroacetic acid (0.5 mL). The reaction mixture was stirred at rt for 1 h. The reaction mixture was concentrated under reduced pressure. The crude product was purified by reverse phase HPLC to afford 2-amino-4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-7-fluorobenzo[b]thiophene-3-carbonitrile as bis(2,2,2-trifluoroacetate) and as yellow solid (13 mg, 0.01 mmol, 26% yield). m/z (ESI): 669.2 (M+H)⁺. ¹H NMR (400 MHZ, METHANOL-d₄) δ ppm 7.95-7.99 (m, 1H), 7.23-7.27 (m, 1H), 7.07 (t, J=8.9 Hz, 1H), 5.45-5.68 (m, 1H), 4.55-4.73 (m, 2H), 4.25-4.33 (m, 1H), 3.82-4.06 (m, 7H), 3.44-3.53 (m, 1H), 2.54-2.83 (m, 2H), 2.25-2.48 (m, 4H), 2.11-2.24 (m, 2H), 1.67-1.87 (m, 6H).

### 2-Amino-4-(6-chloro-4-(5,6-dihydro-2H-pyran-3-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile bis(2,2,2-trifluoroacetate) (Example 70)

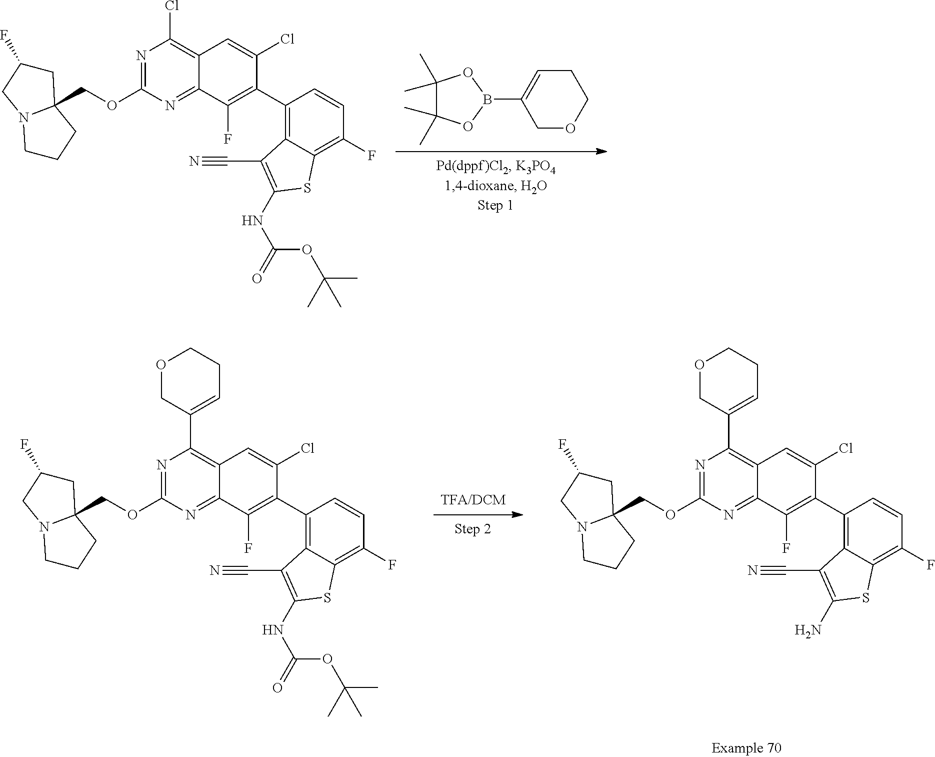 [SMILES: CC(C)(C)OC(=O)Nc1sc2c(F)ccc(-c3c(Cl)cc4c(Cl)nc(OC[C@@]56CCCN5C[C@H](F)C6)nc4c3F)c2c1C#N ; CC1(C)OB(C2=CCCOC2)OC1(C)C ; CC(C)(C)OC(=O)Nc1sc2c(F)ccc(-c3c(Cl)cc4c(C5=CCCOC5)nc(OC[C@@]56CCCN5C[C@H](F)C6)nc4c3F)c2c1C#N ; N#Cc1c(N)sc2c(F)ccc(-c3c(Cl)cc4c(C5=CCCOC5)nc(OC[C@@]56CCCN5C[C@H](F)C6)nc4c3F)c12]

Step 1: tert-Butyl (4-(6-chloro-4-(5,6-dihydro-2H-pyran-3-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate. In a red-capped vial was charged with the mixture of tert-butyl (3-cyano-4-(4,6-dichloro-8-fluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate (21 mg, 0.032 mmol), 2-(5,6-dihydro-2h-pyran-3-yl)-4,4,5,5-tetramethyl-1,3,2-dioxaborolane (13 mg, 0.063 mmol), [1,1′-bis(diphenylphosphino)ferrocene] dichloropalladium (ii) (4.6 mg, 6.32 μmol), and potassium phosphate tribasic (20 mg, 0.095 mmol) in 1,4-dioxane (0.8 mL) and water (0.2 mL). The reaction mixture was heated at 90° C. for 1 h. After cooling to rt, the mixture was purified by preparative HPLC to afford tert-butyl (4-(6-chloro-4-(5,6-dihydro-2H-pyran-3-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate as yellow solid. m/z (ESI, +ve ion): 711.7 (M+H)⁺.
Step 2: 2-Amino-4-(6-chloro-4-(5,6-dihydro-2H-pyran-3-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile bis(2,2,2-trifluoroacetate). tert-Butyl (4-(6-chloro-4-(5,6-dihydro-2H-pyran-3-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate from previous step was dissolved in dichloromethane (0.5 mL), trifluoroacetic acid (0.5 mL) was added and the reaction mixture was stirred at rt for 1 h, then concentrated and purified by reverse phase HPLC to afford 2-amino-4-(6-chloro-4-(5,6-dihydro-2H-pyran-3-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile bis(2,2,2-trifluoroacetate) (3.8 mg, 4.52 μmol, 14% yield for two steps) as yellow solid. ¹H NMR (400 MHz, METHANOL-d₄) δ ppm 8.25 (d, J=1.7 Hz, 1H), 7.26 (dd, J=8.4, 5.0 Hz, 1H), 7.09 (dd, J=9.3, 8.5 Hz, 1H), 6.64-6.68 (m, 1H), 5.49-5.76 (m, 1H), 4.65-4.80 (m, 4H), 3.88-4.09 (m, 5H), 3.47-3.56 (m, 1H), 2.61-2.88 (m, 2H), 2.34-2.59 (m, 5H), 2.13-2.31 (m, 1H). m/z (ESI): 611.8 (M+H)⁺.

### 2-Amino-4-(6-chloro-4-cyclopropyl-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile bis(2,2,2-trifluoroacetate) (Example 71)

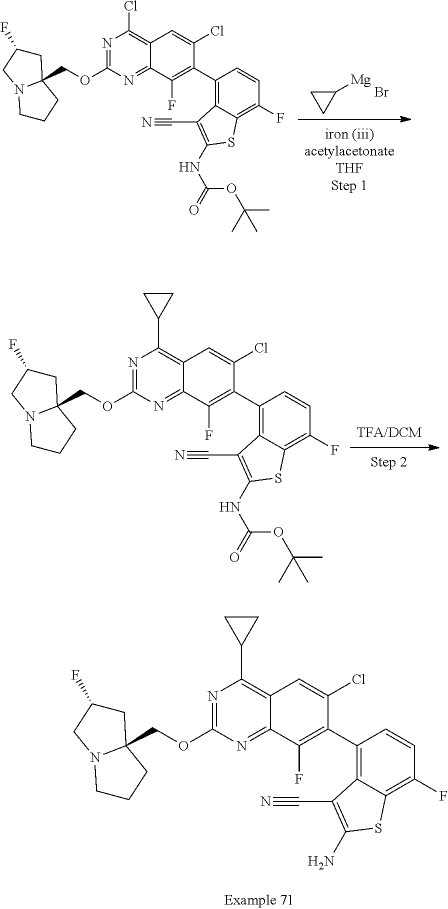

Step 1: tert-Butyl (4-(6-chloro-4-cyclopropyl-8-fluoro-2-(((2,7aS)-2-fluorotetrahydro-1H-pyrrolizin-7a(5H)-yl)methoxy)quinazolin-7-yl)-3-cyano-7-fluorobenzo[b]thiophen-2-yl)carbamate. To a stirred solution of tert-butyl (3-cyano-4-(4,6-dichloro-8-fluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate (20 mg, 0.030 mmol) in tetrahydrofuran (1.0 mL) at 0° C. was added iron (III) acetylacetonate (5.3 mg, 0.015 mmol), followed by cyclopropylmagnesium bromide (1 M solution in THF, 0.06 mL, 0.060 mmol). The resulting mixture was stirred at 0° C. for 1 h, quenched with water, and extracted with EtOAc. The organic layer was dried (Na₂SO₄), concentrated. The residue was purified by reverse phase HPLC to afford tert-butyl (4-(6-chloro-4-cyclopropyl-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (10 mg, 0.015 mmol, 50% yield) as white solid. m/z (ESI): 670.0 (M+H)⁺.
Step 2:2-Amino-4-(6-chloro-4-cyclopropyl-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile bis(2,2,2-trifluoroacetate). tert-Butyl (4-(6-chloro-4-cyclopropyl-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (10 mg, 0.015 mmol) was dissolved in dichloromethane (0.5 mL), trifluoroacetic acid (0.5 mL) was added and the reaction mixture was stirred at rt for 1 h. The reaction mixture was concentrated and purified by reverse phase HPLC to afford 2-amino-4-(6-chloro-4-cyclopropyl-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile bis(2,2,2-trifluoroacetate) (7.8 mg, 9.8 μmol, 65% yield) as yellow solid. 1H NMR (400 MHZ, METHANOL-d₄) δ ppm 8.50 (s, 1H), 7.25 (t, J-6.5 Hz, 1H), 7.05-7.12 (m, 1H), 5.48-5.72 (m, 1H), 4.64 ˜ 4.83 (m, 2H), 3.87-4.09 (m, 3H), 3.45-3.56 (m, 1H), 2.98-3.05 (m, 1H), 2.56-2.82 (m, 2H), 2.32-2.51 (m, 3H), 2.12-2.26 (m, 1H), 1.29-1.57 (m, 4H). m/z (ESI): 570.2 (M+H)⁺.

### Biological Evalution

Provided in this section is the biological evaluation of the specific examples provided herein.

### KRAS G12D TR-FRET Assay

Compounds of interest were prepared in a dose-response titration in DMSO, and 80 nL were added via Labcyte Echo to each well of a 384-well plate (Perkin Elmer 6008280). The His-tagged KRAS G12D protein (Amgen) was diluted to 20 nM in Assay Buffer (20 mM HEPES, pH 7.4, 10 mM MgCl₂, 50 mM NaCl, 0.1% BSA, 0.01% Tween-20, 10 μM GDP) and 2 μL was added to the appropriate wells of the 384-well plate. The plate was incubated for 30 minutes at rt. Biotinylated KRPep-2d substrate (Amgen) was diluted to 20 DM in Assay Buffer and 2 AL was added to all wells and incubated for 1 hour at room temperature. Detection Reagent (0.4 nM LANCE Eu-W1024 Anti-6×His (Perkin Elmer AD0401), 5 nM streptavidin-d2 (Cisbio 610SADLA)) was prepared in Assay Buffer, then 4 AL was added to the plate and incubated for 1 h at rt. Plates were read using PerkinElmer En Vision (ex: 320 nm, em1: 665 nm, em2: 615 nm) and em1/em2 data was used to generate curve fits using a 4-parameter logistic model to calculate IC₅₀ values.

### KRAS G12D Coupled Nucleotide Exchange Assay

Purified GDP-bound KRAS protein (aa 1-169), containing both G12D and C118A amino acid substitutions and an N-terminal His-tag, was pre-incubated in assay buffer (25 mM HEPES pH 7.4, 10 mM MgCl₂, and 0.01% Triton X-100) with a compound dose-response titration for 2 h. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 10 min. The assay plates were then read on a PerkinElmer En Vision Multilabel Reader, using AlphaScreen® technology, and data were analyzed using a 4-parameter logistic model to calculate IC₅₀ values.

### Phospho-ERK1/2 MSD Assay

AsPC-1 (ATCC® CRL-1682TM) cells were cultured in RPMI 1640 Medium (ThermoFisher Scientific 11875093) containing 10% fetal bovine serum (ThermoFisher Scientific 16000044) and 1× penicillin-streptomycin-glutamine (ThermoFisher Scientific 10378016). Sixteen hours prior to compound treatment, AsPC-1 cells were seeded in 96-well cell culture plates at a density of 25,000 cells/well and incubated at 37° C., 5% CO₂. A compound dose-response titration was diluted in growth media, added to appropriate wells of a cell culture plate, and then incubated at 37° C., 5% CO₂ for 2 hours. Following compound treatment, cells were washed with ice-cold Dulbecco's phosphate-buffered saline, no Ca²⁺ or Mg²⁺ (ThermoFisher Scientific 14190144), and then lysed in RIPA buffer (50 mM Tris-HCl pH 7.5, 1% Igepal, 0.5% sodium deoxycholate, 150 mM NaCl, and 0.5% sodium dodecyl sulfate) containing protease inhibitors (Roche 4693132001) and phosphatase inhibitors (Roche 4906837001). Phosphorylation of ERK1/2 in compound-treated lysates was assayed using Phospho-ERK1/2 Whole Cell Lysate kits (Meso Scale Discovery K151DWD) according to the manufacturer's protocol. Assay plates were read on a Meso Scale Discovery Sector Imager 6000, and data were analyzed using a 4-parameter logistic model to calculate IC₅₀ values.

TABLE 8
Biochemical and cellular activity of examples

| | KRAS G12D | KRAS G12D | 2 h p-ERK |
|---|---|---|---|
| | TR-FRET | Coupled | (AsPC- |
| | IC₅₀ | Exchange IC₅₀ | 1 cells), |
| Ex.# | (μM) | (μM) | IC₅₀ (μM) |
| 1 | 0.001 | 0.001 | 0.052 |
| 2 | 0.034 | 0.046 | NT |
| 3 | 0.004 | 0.007 | 0.105 |
| 4 | 0.004 | 0.006 | 0.041 |
| 5 | 0.003 | 0.004 | 0.097 |
| 6 | 0.005 | 0.005 | 0.143 |
| 7 | 0.004 | 0.006 | 0.264 |
| 8 | 0.002 | 0.004 | 0.015 |
| 9 | 0.060 | 0.041 | NT |
| 10 | 0.044 | 0.036 | NT |
| 11 | 0.007 | 0.007 | 0.677 |
| 12 | 0.006 | 0.006 | 1.810 |
| 13 | 0.005 | 0.005 | 2.210 |
| 14 | 0.004 | 0.004 | 0.729 |
| 15 | 0.003 | 0.004 | 0.296 |
| 16 | 0.002 | 0.003 | 0.076 |
| 17 | 0.003 | 0.006 | 0.768 |
| 18 | 0.002 | 0.002 | 0.013 |
| 19 | 0.164 | 0.094 | NT |
| 20 | 0.022 | 0.026 | 1.020 |
| 21 | 0.010 | 0.008 | 0.532 |
| 22 | 0.002 | 0.002 | 0.013 |
| 23 | 0.044 | 0.042 | NT |
| 24 | 0.003 | 0.002 | 0.185 |
| 25 | 0.003 | 0.003 | 0.492 |
| 26 | 0.006 | 0.007 | 0.601 |
| 27 | 0.002 | 0.002 | 0.043 |
| 28 | 0.003 | 0.005 | 0.097 |
| 29 | 0.004 | 0.006 | 0.081 |
| 30 | 0.0004 | 0.0008 | 0.212 |
| 31 | 0.001 | 0.001 | 0.187 |
| 32 | 0.002 | 0.002 | 0.079 |
| 33 | 0.003 | 0.003 | 0.285 |
| 34 | 0.004 | 0.004 | 0.093 |
| 35 | 0.004 | 0.005 | 0.199 |
| 36 | 0.005 | 0.006 | 0.205 |
| 37 | 0.008 | 0.010 | 0.859 |
| 38 | 0.010 | 0.010 | 0.781 |
| 39 | 0.010 | 0.011 | 0.283 |
| 40 | 0.019 | 0.026 | 1.680 |
| 41 | 0.003 | 0.004 | 0.288 |
| 42 | 0.008 | 0.010 | 0.402 |
| 43 | 0.015 | 0.018 | 1.160 |
| 44 | 0.002 | 0.003 | 0.032 |
| 45 | 0.006 | 0.005 | 0.185 |
| 46 | 0.006 | 0.003 | 0.148 |
| 47 | 0.017 | 0.007 | 1.110 |
| 48 | 0.045 | 0.021 | 3.060 |
| 49 | 0.004 | 0.004 | 0.252 |
| 50 | 0.007 | 0.007 | 0.307 |
| 51 | 0.002 | 0.002 | 0.089 |
| 52 | 0.008 | 0.007 | 0.505 |
| 53 | 0.001 | 0.001 | 0.119 |
| 54 | 0.014 | 0.013 | 1.090 |
| 55 | 0.005 | 0.008 | 0.102 |
| 56 | 0.017 | 0.030 | NT |
| 57 | 0.003 | 0.003 | 0.061 |
| 58 | 0.003 | 0.001 | 0.078 |
| 59 | 0.005 | 0.002 | 0.062 |
| 60 | 0.008 | 0.007 | 0.292 |
| 61 | 0.005 | 0.003 | 0.050 |
| 62 | 0.003 | 0.001 | 0.025 |
| 63 | 0.004 | 0.003 | >10 |
| 64 | 0.025 | 0.028 | 1.520 |
| 65 | 0.012 | 0.006 | 0.33 |
| 66 | 0.024 | 0.016 | 0.586 |
| 67 | 0.061 | 0.034 | NT |
| 68 | 0.144 | 0.091 | NT |
| 69 | 0.139 | 0.071 | NT |
| 70 | 0.034 | 0.019 | 0.845 |
| 71 | 0.011 | 0.005 | 0.185 |

NT: not tested.

### REFERENCES

All references, for example, a scientific publication or patent application publication, cited herein are incorporated herein by reference in their entirety and for all purposes to the same extent as if each reference was specifically and individually indicated to be incorporated by reference in its entirety for all purposes.

What is claimed is:

1. A compound of formula (I): 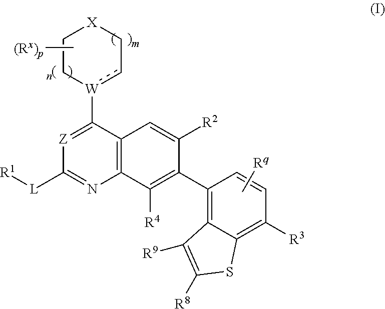
or tautomer thereof, or a pharmaceutically acceptable salt of said compound or said tautomer,
wherein; [symbol]
is a single bond or a double bond;
W is C, CH or N, wherein when W is N, [symbol] is a single bond;
X is a bond, CH₂, O, S, S(O), S(O)(NR^{z}) or S(O)₂;
n is 0, 1, 2, or 3;
m is 0, 1, 2, 3 or 4;
each R^{x} is hydroxyl, halogen, oxo, cyano, C₁₋₄ alkyl, C₁₋₄ haloalkyl, C₁₋₄ alkoxy, —T—R^{y} or two R^{x} taken together with the same carbon or adjacent carbon atoms can form C₃₋₇ cycloalkyl or a 5-7 membered heterocycloalkyl, wherein each C₃₋₇ cycloalkyl or 5-7 membered heterocycloalkyl is further substituted with 0-3 occurrences of R^{y} or two R^{x} taken together with the same carbon atom can form a C₃₋₈ cycloalkyl or 4-7 membered heterocycloalkyl substituted with 0-3 occurrences of R^{y} or two R^{x} taken together with the carbon atoms to which they are attached can form a bridged ring, wherein the bridge atoms are selected from one of the following: —C₁₋₄ alkylene, —C₁₋₄ alkylene-O—, —C₁₋₄ alkylene-O—C₁₋₄ alkylene-, —C₁₋₄ alkylene-S—C₁₋₄ alkylene- or —C₁₋₄ alkylene-S—;
Z is CH, CR′ or N;
R¹ is halogen, cyano or C₁₋₄ alkyl;
L is a bond, —C₁₋₄ alkylene, —O—C₁₋₄ alkylene, —S—C₁₋₄ alkylene, —NR^{z}—, —O— or —S—;
R¹ is hydroxyl, aryl, heteroaryl, C₃₋₈ cycloalkyl or heterocycloalkyl optionally substituted with 0-3 occurrences of R⁵;
R² is hydrogen, halogen, C₁₋₄ alkyl, C₂₋₄ alkenyl or cyano;
R³ hydrogen, halogen, cyano, C₁₋₄ alkyl, C₁₋₄ haloalkyl or C₂₋₄ alkynyl;
R⁴ is hydrogen or halogen;
each R⁵ is halogen, oxo, hydroxyl, amino or C₁₋₄ alkyl;
p is 0, 1, 2 or 3;
R⁸ is amino;
R⁹ is cyano;
T is C₁₋₄ alkylene, —C(O)—, —O—, —S(O)₂— or —S—;
R^{q} is hydrogen, halogen or C₁₋₄ alkyl;
R^{y} is halogen, hydroxyl, oxo, cyano or amino; and
R^{z} is hydrogen or C₁₋₄ alkyl.

2. The compound of claim 1, wherein Z is N.

3. The compound of claim 1, wherein L is —C₁₋₄ alkylene or —O—C₁₋₄ alkylene.

4. The compound of claim 3, wherein R¹ is 7-(hexahydro-1H-pyrrolizine), 2-pyrrolidine or N-morpholinyl substituted with 0-3 occurrences of R⁵.

5. The compound of claim 4, wherein L-R¹ is

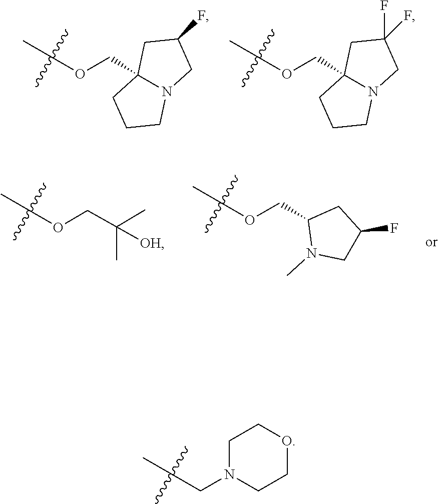

6. The compound of claim 1, wherein W is N.

7. The compound of claim 6, wherein X is O, CH₂, S or S(O)₂.

8. The compound of claim 7, wherein X is O.

9. The compound of claim 7, wherein n is 1 and m is 2, m is 1 and n is 2 or n is 2 and m is 2.

10. The compound of claim 1, wherein 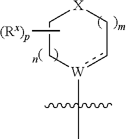
is

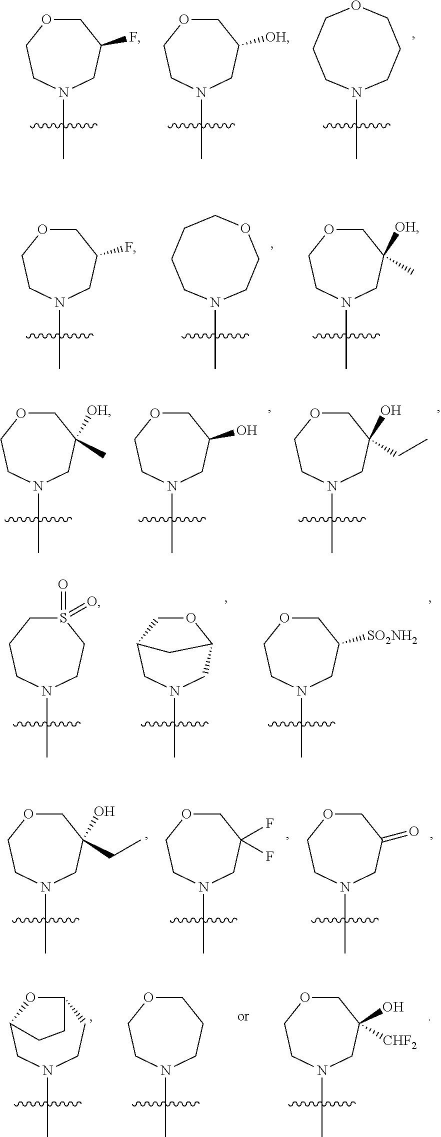

11. The compound of claim 7, wherein X is CH₂.

12. The compound of claim 10, wherein n is 0 and m is 1 or m is 0 and n is 1.

13. The compound of claim 1, wherein 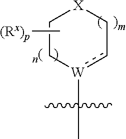
is

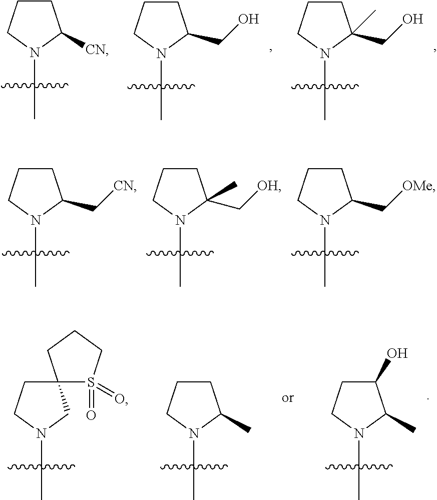

14. The compound of claim 10, wherein n is 1 and m is 1.

15. The compound of claim 1, wherein 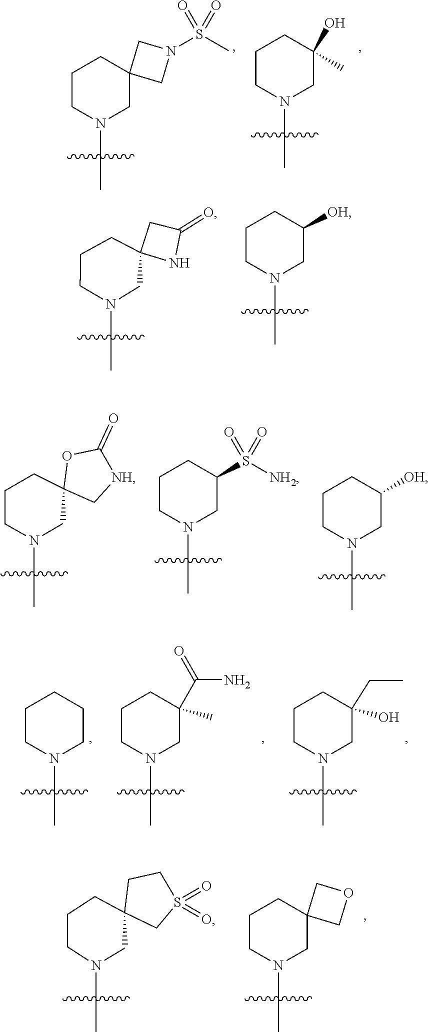 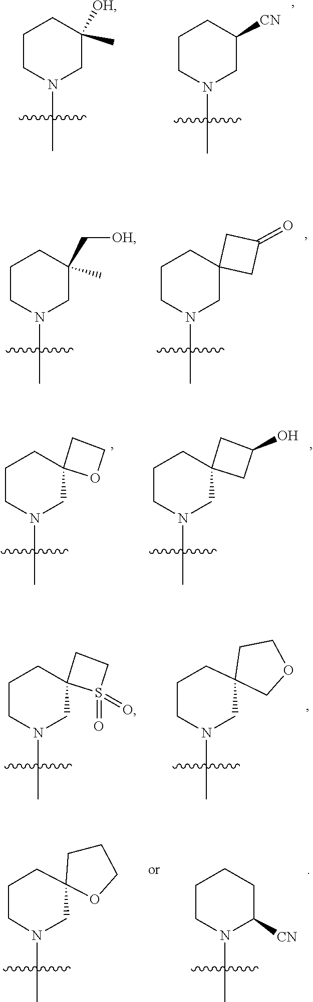
is

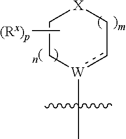

16. The compound of claim 10, wherein n is 1 and m is 2, m is 1 and n is 2 or n is 2 and m is 2.

17. The compound of claim 1, wherein 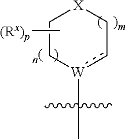
is

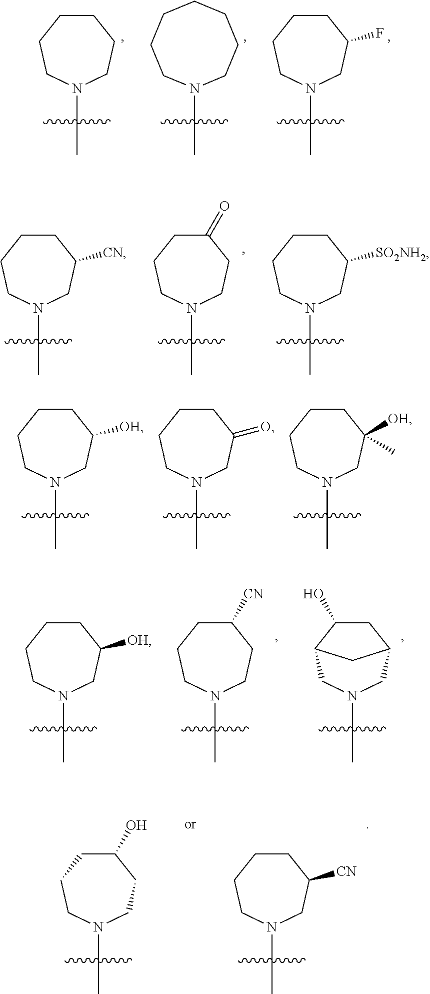

18. The compound of claim 1, wherein W is C and [symbol] is a double bond or W is C and [symbol] is a single bond.

19. The compound of claim 17, wherein X is O or a bond.

20. The compound of claim 1, wherein 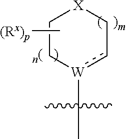
is

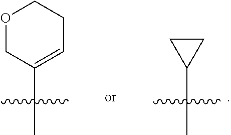

21. The compound of claim 1, wherein R² is halogen, C₁₋₄ alkyl, C₂₋₄ alkenyl or cyano.

22. The compound of claim 1, wherein R⁴ is halogen.

23. The compound of claim 1, wherein R³ is hydrogen or halogen.

24. The compound of claim 1, wherein the compound is selected from one of the following compounds:
2-Amino-4-(6-chloro-8-fluoro-2-(((2R,7aS)-2-fluorotetrahydro-1H-pyrrolizin-7a(5H)-yl)methoxy)-4-(1,5-oxazocan-5-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile;
2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-4-(6-ethyl-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((R)-3-hydroxyazepan-1-yl)quinazolin-7-yl)-7-fluorobenzo[b]thiophene-3-carbonitrile;
2-Amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile;
2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-3-carbonitrile;
2-Amino-4-(6-chloro-8-fluoro-4-(6-fluoro-1,4-oxazepan-4-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(4-oxoazepan-1-yl)quinazolin-7-yl)-7-fluorobenzo[b]thiophene-3-carbonitrile;
2-Amino-4-(6-chloro-8-fluoro-4-(6-fluoro-1,4-oxazepan-4-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((S)-6-hydroxy-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-hydroxy-2,3,6,7-tetrahydro-1H-azepin-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(1,4-oxazocan-4-yl)quinazolin-7-yl)-7-fluorobenzo[b]thiophene-3-carbonitrile;
2-Amino-4-(6-chloro-4-(6,6-difluoro-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((S)-3-hydroxyazepan-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-hydroxy-3-methylazepan-1-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile;
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-4-carbonitrile;
(2S)-1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)pyrrolidine-2-carbonitrile;
2-Amino-4-(6-chloro-8-fluoro-2-(((2R,7aS)-2-fluorotetrahydro-1H-pyrrolizin-7a(5H)-yl)methoxy)-4-(6-oxo-1,4-oxazepan-4-yl)quinazolin-7-yl)-7-fluorobenzo[b]thiophene-3-carbonitrile;
2-Amino-4-(6-chloro-4-(6-(difluoromethyl)-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile; or
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile.

25. The compound of claim 1, wherein the compound is selected from one of the compounds: following
2-Amino-4-(6-chloro-8-fluoro-2-(((2R,7aS)-2-fluorotetrahydro-1H-pyrrolizin-7a(5H)-yl)methoxy)-4-(1,5-oxazocan-5-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile;
2-Amino-4-(6-ethyl-8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)quinazolin-7-yl)-7-fluorobenzo[b]thiophene-3-carbonitrile;
2-Amino-4-(6-chloro-4-(6-ethyl-6-hydroxy-1,4-oxazepan-4-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-4-(6-chloro-8-fluoro-2-(((2R,7aS)-2-fluorotetrahydro-1H-pyrrolizin-7a(5H)-yl)methoxy)-4-((R)-3-hydroxyazepan-1-yl)quinazolin-7-yl)-7-fluorobenzo[b]thiophene-3-carbonitrile;
2-Amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile;
2-Amino-7-fluoro-4-(8-fluoro-2-(((2R,7aS)-2-fluorotetrahydro-1H-pyrrolizin-7a(5H)-yl)methoxy)-4-(6-hydroxy-6-methyl-1,4-oxazepan-4-yl)-6-vinylquinazolin-7-yl)benzo[b]thiophene-3-carbonitrile;
2-Amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile; or
1-(7-(2-Amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl) azepane-3-carbonitrile.

26. A pharmaceutical composition comprising the compound according to any one of claims 1-25 or a pharmaceutically acceptable salt of said compound, and a pharmaceutically acceptable excipient.

27-33. (canceled)

34. A method of treating cancer in a subject in need thereof, the method comprising administering to the subject a therapeutically effective amount of the compound according to any one of to any one of claims 1-25 or a pharmaceutically acceptable salt thereof or a pharmaceutical composition according to claim 26.

35. A method of treating cancer in a subject in need thereof, the method comprising administering to the subject a therapeutically effective amount of the compound according to any one of to any one of claims 1-25 or a pharmaceutically acceptable salt thereof or a pharmaceutical composition according to claim 26, wherein one or more cells express KRAS G12D mutant protein.

36. The method according to claim 34 or 35, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

37. The method according to claim 34 or 35, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

38. The method according to claim 37, wherein the cancer is non-small cell lung cancer.

39. The method according to claim 37, wherein the cancer is colorectal cancer.

40. The method according to claim 37, wherein the cancer is pancreatic cancer.

41. The method according to anyone of claims 34-40, wherein the subject has a cancer that was determined to have one or more cells expressing the KRAS G12D mutant protein prior to administration of the compound or a pharmaceutically acceptable salt thereof.
